SEQUENCE LISTING (197 sequences)

SEQ ID NO: 1 - protein, 662 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEDLKDAQTRITKGSHHHHHHG

SEQ ID NO: 2 - protein, 662 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADPPSYNVFIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEDLKDAQTRITKGSHHHHHHG

SEQ ID NO: 3 - protein, 659 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADPCSHNVFIMADKQKNGIKANFKIRHNIEDGGVQLAYHYQQNTPIGDGPVLLPDNHYLSTQSKLSKDPNEKRDHMVLLEFVTAAGITLGMDELYKGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVDKPRIAATMENAQKGEIMPNIPQMSAFWYAVRTAVINAASGRQTVDEDLKDAQTRITKGSHHHHHHG

SEQ ID NO: 4 - protein, 661 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGGSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGKYDIKDVGDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVDKPRIAATMENAQKGEIMPNIPQMSAFWYAVRTAVINAASGQTVDEDLKDAQTRITKGSHHHHHHG

SEQ ID NO: 5 - protein, 663 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENHLSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEDLKDAQTRITKGSHHHHHHG

SEQ ID NO: 6 - protein, 655 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELGGSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGAKDPRIAATMENAQKGEIMPNIPQMSAFWYAVRTAVINAASGRQTVDEDLKDAQTRITK

SEQ ID NO: 7 - protein, 655 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELGGSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPAKDPRIAATMENAQKGEIMPNIPQMSAFWYAVRTAVINAASGRQTVDEDLKDAQTRITK

SEQ ID NO: 8 - protein, 659 aa, synthetic construct
MRGSHHHHHHGMASMTGGQQMGRDLYDDDDKDRWGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNAATMENAQKGEIMPNIPQMSAFWYAVRTAVINAASGRQTVDEDLKDAQTRITKGSHHHHHHG

SEQ ID NO: 9 - protein, 14 aa, synthetic construct
  VARIANT 8..9: note = any amino acid
PLIAADGXXNVYIM

SEQ ID NO: 10 - protein, 13 aa, synthetic construct
  VARIANT 7..8: note = any amino acid
PLIAADXXNVYIM

SEQ ID NO: 11 - protein, 15 aa, synthetic construct
  VARIANT 9..10: note = any amino acid
PLIAADGGXXNVYIM

SEQ ID NO: 12 - protein, 15 aa, synthetic construct
  VARIANT 8: note = any amino acid
PLIAADGXPNVYIMG

SEQ ID NO: 13 - protein, 15 aa, synthetic construct
  VARIANT 9: note = any amino acid
PLIAADGIXNVYIMG

SEQ ID NO: 14 - protein, 15 aa, synthetic construct
  VARIANT 8: note = any amino acid
PLIAADPXSHNVYIM

SEQ ID NO: 15 - protein, 15 aa, synthetic construct
  VARIANT 7: note = any amino acid
PLIAADXPSHNVYIM

SEQ ID NO: 16 - protein, 15 aa, synthetic construct
  VARIANT 7..8: note = any amino acid
PLIAADXXSHNVYIM

SEQ ID NO: 17 - protein, 15 aa, synthetic construct
  VARIANT 7..8: note = any amino acid
PLIAADXXSHNVFIM

SEQ ID NO: 18 - protein, 15 aa, synthetic construct
  VARIANT 8: note = any amino acid
PLIAADPXSHNVFIM

SEQ ID NO: 19 - protein, 15 aa, synthetic construct
  VARIANT 8: note = any amino acid
PLIAADPXSYNVFIM

SEQ ID NO: 20 - protein, 15 aa, synthetic construct
  VARIANT 7..8: note = any amino acid
PLIAADXXSYNVFIM

SEQ ID NO: 21 - protein, 15 aa, synthetic construct
  VARIANT 8: note = any amino acid
PLIAADPXSYNVFIM

SEQ ID NO: 22 - protein, 15 aa, synthetic construct
  VARIANT 7..8: note = any amino acid
PLIAADXXSYNVFIM

SEQ ID NO: 23 - protein, 15 aa, synthetic construct
  VARIANT 8: note = any amino acid
  VARIANT 10: note = any amino acid
PLIAADPXSXNVYIM

SEQ ID NO: 24 - protein, 15 aa, synthetic construct
  VARIANT 8: note = any amino acid
  VARIANT 11: note = any amino acid
PLIAADPXSHXVYIM

SEQ ID NO: 25 - protein, 15 aa, synthetic construct
  VARIANT 8: note = any amino acid
  VARIANT 12: note = any amino acid
PLIAADPXSHNXYIM

SEQ ID NO: 26 - protein, 15 aa, synthetic construct
  VARIANT 8: note = any amino acid
  VARIANT 13: note = any amino acid
PLIAADPXSHNVXIM

SEQ ID NO: 27 - protein, 16 aa, synthetic construct
  VARIANT 8..9: note = any amino acid
KLEYNFNXXYAFKYEN

SEQ ID NO: 28 - protein, 15 aa, synthetic construct
  VARIANT 8: note = any amino acid
KLEYNFNXYAFKYEN

SEQ ID NO: 29 - protein, 14 aa, synthetic construct
KLEYNFNYAFKYEN

SEQ ID NO: 30 - protein, 14 aa, synthetic construct
  VARIANT 7: note = any amino acid
KLEYNFXYAFKYEN

SEQ ID NO: 31 - protein, 14 aa, synthetic construct
  VARIANT 6..7: note = any amino acid
KLEYNXXYAFKYEN

SEQ ID NO: 32 - protein, 14 aa, synthetic construct
  VARIANT 7: note = any amino acid
KLEYNWXYAFKYEN

SEQ ID NO: 33 - protein, 14 aa, synthetic construct
  VARIANT 6: note = any amino acid
KLEYNXKYAFKYEN

SEQ ID NO: 34 - protein, 16 aa, synthetic construct
  VARIANT 9: note = any amino acid
KLEYNFNPXYAFKYEN

SEQ ID NO: 35 - protein, 16 aa, synthetic construct
  VARIANT 8: note = any amino acid
KLEYNFNXPYAFKYEN

SEQ ID NO: 36 - protein, 15 aa, synthetic construct
  VARIANT 7..8: note = any amino acid
AFKYENXXSHNVYIM

SEQ ID NO: 37 - protein, 16 aa, synthetic construct
  VARIANT 8..9: note = any amino acid
KLEYNFNXXKYDIKDV

SEQ ID NO: 38 - protein, 15 aa, synthetic construct
  VARIANT 7..8: note = any amino acid
KSYEELXXSHNVYIM

SEQ ID NO: 39 - protein, 15 aa, synthetic construct
  VARIANT 8: note = any amino acid
KSYEELPXSHNVYIM

SEQ ID NO: 40 - protein, 15 aa, synthetic construct
  VARIANT 7: note = any amino acid
KSYEELXPSHNVYIM

SEQ ID NO: 41 - protein, 16 aa, synthetic construct
  VARIANT 8..9: note = any amino acid
KLEYNFNXXAKDPRIA

SEQ ID NO: 42 - protein, 16 aa, synthetic construct
  VARIANT 9: note = any amino acid
KLEYNFNPXAKDPRIA

SEQ ID NO: 43 - protein, 16 aa, synthetic construct
  VARIANT 8: note = any amino acid
KLEYNFNXPAKDPRIA

SEQ ID NO: 44 - protein, 15 aa, synthetic construct
  VARIANT 8: note = any amino acid
ELAKDPRXSHNVYIM

SEQ ID NO: 45 - protein, 16 aa, synthetic construct
  VARIANT 8..9: note = any amino acid
ELAKDPRXXSHNVYIM

SEQ ID NO: 46 - protein, 17 aa, synthetic construct
  VARIANT 8..10: note = any amino acid
ELAKDPRXXXSHNVYIM

SEQ ID NO: 47 - protein, 15 aa, synthetic construct
  VARIANT 8: note = any amino acid
KLEYNFNXAATMENA

SEQ ID NO: 48 - protein, 16 aa, synthetic construct
  VARIANT 8..9: note = any amino acid
KLEYNFNXXAATMENA

SEQ ID NO: 49 - protein, 17 aa, synthetic construct
  VARIANT 8..10: note = any amino acid
KLEYNFNXXXAATMENA

SEQ ID NO: 50 - protein, 671 aa, synthetic construct
MRGSHHHHHHGMASMTGGQQMGRDLYDDDDKDRWGSKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYSKEMVSEPPKTFDEMKAIMEKYYDPANEKYGIAWPINAYFISAIAQAFGGSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQGSHHHHHHG

SEQ ID NO: 51 - protein, 671 aa, synthetic construct
MRGSHHHHHHGMASMTGGQQMGRDLYDDDDKDRWGSKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYSKEMVSEPPKTFDEMKAIMEKYYDPANEKYGIAWPINAYFISAIAQAFGGSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNFEYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQGSHHHHHHG

SEQ ID NO: 52 - protein, 662 aa, synthetic construct
MRGSHHHHHHGMASMTGGQQMGRDLYDDDDKDRWGSKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYSKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEISHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNKNDPVIYGFGQAVQHAYLMPKSPKMSAVWGGVDGAINEILQDPQNADIEGILKKYQQEILNNMQGS

SEQ ID NO: 53 - protein, 664 aa, synthetic construct
MRGSHHHHHHGMASMTGGQQMGRDLYDDDDKDRWGSKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYSKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIPPSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNKNDPVIYGFGQAVQHAYLMPKSPKMSAVWGGVDGAINEILQDPQNADIEGILKKYQQEILNNMQGS

SEQ ID NO: 54 - protein, 16 aa, synthetic construct
  VARIANT 7..8: note = any amino acid
AIAQAFXXSHNVYIMA

SEQ ID NO: 55 - protein, 16 aa, synthetic construct
  VARIANT 8: note = any amino acid
AIAQAFPXSHNVYIMA

SEQ ID NO: 56 - protein, 17 aa, synthetic construct
  VARIANT 8..9: note = any amino acid
KLEYNFNXXYYFDDKTE

SEQ ID NO: 57 - protein, 14 aa, synthetic construct
  VARIANT 7..8: note = any amino acid
VLDDPEXXHNVYIM

SEQ ID NO: 58 - protein, 16 aa, synthetic construct
  VARIANT 8..9: note = any amino acid
VLDDPEIXXSHNVYIM

SEQ ID NO: 59 - protein, 14 aa, synthetic construct
  VARIANT 7..8: note = any amino acid
KLEYNFXXNDPVIY

SEQ ID NO: 60 - protein, 16 aa, synthetic construct
  VARIANT 8: note = any amino acid
KLEYNFNXPKNDPVIY

SEQ ID NO: 61 - protein, 16 aa, synthetic construct
  VARIANT 9: note = any amino acid
KLEYNFNPXKNDPVIY

SEQ ID NO: 62 - protein, 556 aa, synthetic construct
MRGSHHHHHHGMASMTGGQQMGRDLYDDDDKDRWGSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILVSHNVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPLNMNFELSDEMKALFKEPNDKALK

SEQ ID NO: 63 - protein, 556 aa, synthetic construct; identical to SEQ ID NO: 62

SEQ ID NO: 64 - protein, 16 aa, synthetic construct
  VARIANT 8: note = any amino acid
FKNPIPPXSHNVYIMA

SEQ ID NO: 65 - protein, 17 aa, synthetic construct
  VARIANT 8..9: note = any amino acid
FKNPIPPXXSHNVYIMA

SEQ ID NO: 66 - protein, 17 aa, synthetic construct
  VARIANT 9: note = any amino acid
FKNPIPPPXSHNVYIMA

SEQ ID NO: 67 - protein, 17 aa, synthetic construct
  VARIANT 8: note = any amino acid
FKNPIPPXPSHNVYIMA

SEQ ID NO: 68 - protein, 17 aa, synthetic construct
  VARIANT 8..9: note = any amino acid
KWFKNPIXXSHNVYIMA

SEQ ID NO: 69 - protein, 16 aa, synthetic construct
  VARIANT 8..9: note = any amino acid
FKNPIPPXXNVYIMAD

SEQ ID NO: 70 - protein, 16 aa, synthetic construct
  VARIANT 8..9: note = any amino acid
KWFKNPIXXNVYIMAD

SEQ ID NO: 71 - protein, 15 aa, synthetic construct
  VARIANT 8: note = any amino acid
KLEYNFNXKNLNMNF

SEQ ID NO: 72 - protein, 16 aa, synthetic construct
  VARIANT 8..9: note = any amino acid
KLEYNFNXXKNLNMNF

SEQ ID NO: 73 - protein, 16 aa, synthetic construct
  VARIANT 8: note = any amino acid
KLEYNFNXPKNLNMNF

SEQ ID NO: 74 - protein, 16 aa, synthetic construct
  VARIANT 9: note = any amino acid
KLEYNFNPXKNLNMNF

SEQ ID NO: 75 - protein, 14 aa, synthetic construct
  VARIANT 8..9: note = any amino acid
GHKLEYNXXLNMNF

SEQ ID NO: 76 - protein, 14 aa, synthetic construct
  VARIANT 8..9: note = any amino acid
KLEYNFNXXLNMNF

SEQ ID NO: 77 - protein, 567 aa, synthetic construct
MHHHHHHGSEEQEKALNFGIISTESQQNLKPQWTPFLQDMEKKLGVKVNAFFAPDYAGIIQGMRFNKVDIAWYGNLSAMEAVDRANGQVFAQTVAADGSSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNPGYWSVLIVNKDSPINNLNDLLAKRKDLTFGNGDPNSTSGFLVPGYYVFAKNINISASDFKRTVNAGHETNALAVANKQVDVATNNTENLDKLKTSAPEKLKELKVIWKSPLIPGDPIVWRKNLSETTKDKIYDFFMNYGKTPEEKAVLERLGWAPFRASSDLQLVPIRQLALFKEMQSVKDNKGLNEQDKLAKTTAIQAQLDDLDRLNNALSAMSSVSKAVQ

SEQ ID NO: 78 - protein, 566 aa, synthetic construct
MHHHHHHGSEEQEKALNFGIISTESQQNLKPQWTPFLQDMEKKLGVKVNAFFAPDYAGIIQGMRFNKVDIAWYGNLSAMEAVDRANGQVFAQTVAADADNVYIMADKQRNGIKANFKIRHNIEDGGVQLAYHYQQNTPIGDGPVLLPDNHYLSTQSKLSKSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNPGYWSVLIVNKDSPINNLNDLLAKRKDLTFGNGDPNSTSGFLVPGYYVFAKNNISAGKTPEEKAVLERLGWAPFRASSDLQLVPIRQLALFKEMQSVKDNKGLNEQDKLAKTTAIQAQLDDLDRRNNARSAMSSVSNYGKTPEEKAVLERLGWAPFRASSDLQLVPIRQLALFKEMQSVKDNKGLNEQDKLAKTTAIQAQLDDLDRLNNALSAMSSVSKAVQ

SEQ ID NO: 79 - protein, 16 aa, synthetic construct
QTVAADGSSHNVYIMA

SEQ ID NO: 80 - protein, 16 aa, synthetic construct
  VARIANT 7..8: note = any amino acid
QTVAADXXSHNVYIMA

SEQ ID NO: 81 - protein, 16 aa, synthetic construct
  VARIANT 7: note = any amino acid
QTVAADXPSHNVYIMA

SEQ ID NO: 82 - protein, 16 aa, synthetic construct
  VARIANT 8: note = any amino acid
QTVAADPXSHNVYIMA

SEQ ID NO: 83 - protein, 14 aa, synthetic construct
  VARIANT 7..8: note = any amino acid
QTVAADXXNVYIMA

SEQ ID NO: 84 - protein, 16 aa, synthetic construct
  VARIANT 7..8: note = any amino acid
QTVAADXXSHNVYIMA

SEQ ID NO: 85 - protein, 16 aa, synthetic construct
  VARIANT 7..8: note = any amino acid
VFQTVAXXSHNVYIMA

SEQ ID NO: 86 - protein, 16 aa, synthetic construct
HKLEYNFNPGYWSVLI

SEQ ID NO: 87 - protein, 18 aa, synthetic construct
  VARIANT 9..10: note = any amino acid
HKLEYNFNXXPGYWSVLI

SEQ ID NO: 88 - protein, 16 aa, synthetic construct
  VARIANT 7..8: note = any amino acid
HKLEYNXXPGYWSVLI

SEQ ID NO: 89 - protein, 16 aa, synthetic construct
  VARIANT 9..10: note = any amino acid
HKLEYNFNXXYWSVLI

SEQ ID NO: 90 - protein, 16 aa, synthetic construct
  VARIANT 10: note = any amino acid
HKLEYNFNPXYWSVLI

SEQ ID NO: 91 - protein, 678 aa, synthetic construct
MRGSHHHHHHGMASMTGGQQMGRDLYDDDDKDRWGSKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYGGSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPNAYGQSAMRDWRSNRIVGSLVHGAVAPESFMSQFGTVMEIFLQTRNPQAAANAAQAIADQVGLGRLGQ

SEQ ID NO: 92 - protein, 678 aa, synthetic construct
MRGSHHHHHHGMASMTGGQQMGRDLYDDDDKDRWGSKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYPASHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPNAYGQSAMRDWRSNRIVGSLVHGAVAPESFMSQFGTVMEIFLQTRNPQAAANAAQAIADQVGLGRLGQ

SEQ ID NO: 93 - protein, 678 aa, synthetic construct
MRGSHHHHHHGMASMTGGQQMGRDLYDDDDKDRWGSKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWARAVLKTRMLGGDPPDTFQVAAGMELIGTWVVANRMEDLSALFRQEGWLQAFPKGLIDLISYKGGIWSVPVNIHRSNVMWAKNRQNAINWLRLVGSKEGQDTFNPLKGSIAARLDSDPSKYGGSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPNAYGQSAMRDWRSNRIVGSLVHGAVAPESFMSQFGTVMEIFLQTRNPQAAANAAQAIADQVGLGRLGQ

SEQ ID NO: 94 - protein, 678 aa, synthetic construct
MRGSHHHHHHGMASMTGGQQMGRDLYDDDDKDRWGSKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYGGSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPNAYGQSAMRDWRSNRIVGSLVAGAVAPESFMSQFGTVMEIFLQTRNPQAAANAAQAIADQVGLGRLGQ

SEQ ID NO: 95 - protein, 16 aa, synthetic construct
  VARIANT 8..9: note = any amino acid
DSDPSKYXXSHNVYIM

SEQ ID NO: 96 - protein, 16 aa, synthetic construct
  VARIANT 9: note = any amino acid
DSDPSKYPXSHNVYIM

SEQ ID NO: 97 - protein, 16 aa, synthetic construct
  VARIANT 8: note = any amino acid
DSDPSKYXPSHNVYIM

SEQ ID NO: 98 - protein, 16 aa, synthetic construct
  VARIANT 8..9: note = any amino acid
RLDSDPSXXSHNVYIM

SEQ ID NO: 99 - protein, 14 aa, synthetic construct
  VARIANT 8..9: note = any amino acid
DSDPSKYXXNVYIM

SEQ ID NO: 100 - protein, 16 aa, synthetic construct
  VARIANT 8..9: note = any amino acid
KLEYNFNXXNAYGQSA

SEQ ID NO: 101 - protein, 16 aa, synthetic construct
  VARIANT 7..8: note = any amino acid
KLEYNFXXPNAYGQSA

SEQ ID NO: 102 - protein, 16 aa, synthetic construct
  VARIANT 8..9: note = any amino acid
GHKLEYNXXNAYGQSA

SEQ ID NO: 103 - protein, 16 aa, synthetic construct
  VARIANT 8: note = any amino acid
KLEYNFNXPNAYGQSA

SEQ ID NO: 104 - protein, 16 aa, synthetic construct
  VARIANT 9: note = any amino acid
KLEYNFNPXNAYGQSA

SEQ ID NO: 105 - protein, 396 aa, Escherichia coli
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 106 - protein, 434 aa, Pyrococcus furiosus
MRRATYAFALLAILVLGVVASGCIGGGTTTPTQTSPATQPTTTQTPTQTETQAVECGSGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYNKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQG

SEQ ID NO: 107 - protein, 338 aa, Escherichia coli
MNAKIIASLAFTSMFSLSTLLNPAYAEEQEKALNFGIISTESQQNLKPQWTPFLQDMEKKLGVKVNAFFAPDYAGIIQGMRFNKVDIAWYGNLSAMEAVDRANGQVFAQTVAADGSPGYWSVLIVNKDSPINNLNDLLAKRKDLTFGNGDPNSTSGFLVPGYYVFAKNNISASDFKRTVNAGHETNALAVANKQVDVATNNTENLDKLKTSAPEKLKELKVIWKSPLIPGDPIVWRKNLSETTKDKIYDFFMNYGKTPEEKAVLERLGWAPFRASSDLQLVPIRQLALFKEMQSVKDNKGLNEQDKLAKTTAIQAQLDDLDRLNNALSAMSSVSKAVQ

SEQ ID NO: 108 - protein, 302 aa, Escherichia coli
MQLRKPATAILALALSAGLAQADDAAPAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDALLAGERAKAKKPDNWEIVGKPQSQEAYGCMLRKDDPQFKKLMDDTIAQVQTSGEAEKWFDKWFKNPIPPKNLNMNFELSDEMKALFKEPNDKALN

SEQ ID NO: 109 - protein, 414 aa, Thermus thermophilus
MRKWLLAIGMVLGLSALAQGGKLEIFSWWAGDEGPALEALIRLYKQKYPGVEVINATVTGGAGVNARAVLKTRMLGGDPPDTFQVHAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYNAYGQSAMRDWRSNRIVGSLVHGAVAPESFMSQFGTVMEIFLQTRNPQAAANAAQAIADQVGLGRLGQ

SEQ ID NO: 110 - protein, 318 aa, Rhizobium meliloti
MIRTLSLKFMLAGAVCMATLTAGSAFAAEPESCGTVRFSDVGWTDITATTATATTILEALGYETDVKVLSVPVTYTSLKNKDIDVFLGNWMPTMEADIAPYREDKSVETVRENLAGAKYTLATNAKGAELGIKDFKDIAAHKDELDGKIYGIEPGNDGNRLIIDMVEKGTFDLKGFEVVESSEQGMLAQVARAEKSGDPIVFLGWEPHPMNANFKLTYLSGGDDVFGPNYGGATVHTNVRAGYTTECPNVGKLLQNLSFSLQMENEIMGKILNDGEDPEKAAAAWLKDNPQSIEPWLSGVATKDGGDGLAAVKAALGL

SEQ ID NO: 111 - protein, 249 aa, Geobacillus stearothermophilus
MGGGRSTETSSSSGGDGGATKKKVVVGTDAAFAPFEYMQKGKIVGFDVDLLDAVMKAAGLDYELKNIGWDPLFASLQSKEVDMGISGITITDERKQSYDFSDPYFEATQVILVKQGSPVKNALDLKGTIGVQNATTGQEAAEKLFGKGPHIKKFETTVVAIMELLNGGVDAVITDNAVANEYVKNNPNKKLQVIEDPKNFASEYYGMIFPKNSELKAKVDEALKNVINSGKYTEIYKKWFGKEPKLDRL

SEQ ID NO: 112 - protein, 370 aa, Agrobacterium tumefaciens
MKKSLLSAVALTAMVAFGGSAWADVVIAVGAPLTGPNAAFGAQIQKGAEQAAKDINAAGGINGEQIKIVLGDDVSDPKQGISVANKFVADGVKFVVGHFNSGVSIPASEVYAENGILEITPAATNPVFTERGLWNTFRTCGRDDQQGGIAGKYLADHFKDAKVAIIHDKTPYGQGLADETKKAANAAGVTEVMYEGVNVGDKDFSALISKMKEAGVSIIYWGGLHTEAGLIIRQAADQGLKAKLVSGDGIVSNELASIAGDAVEGTLNTFGPDPTLRPENKELVEKFKAAGFNPEAYTLYSYAAMQAIAGAAKAAGSVEPEKVAEALKKGSFPTALGEISFDEKGDPKLPGYVMYEWKKGPDGKFTYIQQ

SEQ ID NO: 113 - protein, 367 aa, Escherichia coli
MNIKGKALLAGCIALAFSNMALAEDIKVAVVGAMSGPVAQYGDQEFTGAEQAVADINAKGGIKGNKLQIVKYDDACDPKQAVAVANKVVNDGIKYVIGHLCSSSTQPASDIYEDEGILMITPAATAPELTARGYQLILRTTGLDSDQGPTAAKYILEKVKPQRIAIVHDKQQYGEGLARAVQDGLKKGNANVVFFDGITAGEKDFSTLVARLKKENIDFVYYGGYHPEMGQILRQARAAGLKTQFMGPEGVANVSLSNIAGESAEGLLVTKPKNYDQVPANKPIVDAIKAKKQDPSGAFVWTTYAALQSLQAGLNQSDDPAEIAKYLKANSVDTVMGPLTWDEKGDLKGFEFGVFDWHANGTATDAK

SEQ ID NO: 114 - protein, 677 aa, synthetic construct
MRGSHHHHHHGMASMTGGQQMGRDLYDDDDKDRWGSKLEIFSWWAGDEGPALEALIRLYKQKYPGVEVINATVTGGAGVNARAVLKTRMLGGDPPDTFQVAAGMELIGTWVVANRMEDLSALFRQEGWLQAFPKGLIDLISYKGGIWSVPVNIHRSNVMWYLPAKLKEWGVNPPRTWEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMVSDSFGLPKGAKNRQNAINWLRLVGSKEGQDTFNPLKGSIAARLDSDPSKYPASHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPNAYGQSAMRDWRSNRIVGSLVAGAVAPESFMSQFGTVMEIFLQTRNPQAAANAAQAIADQVGLGRLGQ

SEQ ID NO: 115 - protein, 7 aa, synthetic construct
  VARIANT 1: note = any amino acid
XPSHNVY

SEQ ID NO: 116 - protein, 7 aa, synthetic construct
  VARIANT 1..2: note = any amino acid
XXSHNVY

SEQ ID NO: 117 - protein, 7 aa, synthetic construct
  VARIANT 1..2: note = any amino acid
XXSHNVF

SEQ ID NO: 118 - protein, 7 aa, synthetic construct
  VARIANT 2: note = any amino acid
PXSHNVF

SEQ ID NO: 119 - protein, 7 aa, synthetic construct
  VARIANT 2: note = any amino acid
PXSYNVF

SEQ ID NO: 120 - protein, 7 aa, synthetic construct
  VARIANT 1..2: note = any amino acid
XXSYNVF

SEQ ID NO: 121 - protein, 7 aa, synthetic construct
  VARIANT 2: note = any amino acid
PXSYNVF

SEQ ID NO: 122 - protein, 7 aa, synthetic construct
  VARIANT 1..2: note = any amino acid
XXSYNVF

SEQ ID NO: 123 - protein, 7 aa, synthetic construct
  VARIANT 2: note = any amino acid
  VARIANT 4: note = any amino acid
PXSXNVY

SEQ ID NO: 124 - protein, 7 aa, synthetic construct
  VARIANT 2: note = any amino acid
  VARIANT 5: note = any amino acid
PXSHXVY

SEQ ID NO: 125 - protein, 7 aa, synthetic construct
  VARIANT 2: note = any amino acid
  VARIANT 6: note = any amino acid
PXSHNXY

SEQ ID NO: 126 - protein, 7 aa, synthetic construct
  VARIANT 2: note = any amino acid
  VARIANT 7: note = any amino acid
PXSHNVX

SEQ ID NO: 127-133 - skipped (7 entries)

SEQ ID NO: 134 - protein, 5 aa, synthetic construct
  VARIANT 4: note = any amino acid
FNPXY

SEQ ID NO: 135 - protein, 5 aa, synthetic construct
  VARIANT 3: note = any amino acid
FNXPY

SEQ ID NO: 136-146 - skipped (11 entries)

SEQ ID NO: 147 - protein, 4 aa, synthetic construct
NPCK

SEQ ID NO: 148 - protein, 5 aa, synthetic construct
  VARIANT 3: note = any amino acid
PPXSH

SEQ ID NO: 149 - protein, 6 aa, synthetic construct
  VARIANT 3..4: note = any amino acid
PPXXSH

SEQ ID NO: 150 - protein, 6 aa, synthetic construct
  VARIANT 4: note = any amino acid
PPPXSH

SEQ ID NO: 151 - protein, 6 aa, synthetic construct
  VARIANT 3: note = any amino acid
PPXPSH

SEQ ID NO: 152-153 - skipped (2 entries)

SEQ ID NO: 154 - protein, 5 aa, synthetic construct
  VARIANT 3: note = any amino acid
FNXKN

SEQ ID NO: 155 - protein, 6 aa, synthetic construct
  VARIANT 3..4: note = any amino acid
FNXXKN

SEQ ID NO: 156 - protein, 6 aa, synthetic construct
  VARIANT 3: note = any amino acid
FNXPKN

SEQ ID NO: 157 - protein, 6 aa, synthetic construct
  VARIANT 4: note = any amino acid
FNPXKN

SEQ ID NO: 158 - skipped

SEQ ID NO: 159 - protein, 7 aa, synthetic construct
NADGSSH

SEQ ID NO: 160 - protein, 6 aa, synthetic construct
  VARIANT 3..4: note = any amino acid
ADXXSH

SEQ ID NO: 161 - protein, 6 aa, synthetic construct
  VARIANT 3: note = any amino acid
ADXPSH

SEQ ID NO: 162 - protein, 6 aa, synthetic construct
  VARIANT 4: note = any amino acid
ADPXSH

SEQ ID NO: 163 - skipped

SEQ ID NO: 164 - protein, 6 aa, synthetic construct
  VARIANT 3..4: note = any amino acid
ADXXSH

SEQ ID NO: 165 - protein, 4 aa, synthetic construct
FNPG

SEQ ID NO: 166 - protein, 6 aa, synthetic construct
  VARIANT 3..4: note = any amino acid
FNXXPG

SEQ ID NO: 167-169 - skipped (3 entries)

SEQ ID NO: 170 - protein, 6 aa, synthetic construct
  VARIANT 3..4: note = any amino acid
KYXXSH

SEQ ID NO: 171 - protein, 6 aa, synthetic construct
  VARIANT 4: note = any amino acid
KYPXSH

SEQ ID NO: 172 - protein, 6 aa, synthetic construct
  VARIANT 3: note = any amino acid
KYXPSH

SEQ ID NO: 173-175 - skipped (3 entries)

SEQ ID NO: 176 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILVSHNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNNPLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 177 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDSLLAGERAKAKKPDNWEIVGKPQSQEAYGCMLRKDDPQFKKLMDDTIAQVQTSGEAEKWFDKWFKNPILVSHNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNNPLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 178 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITAQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILVSHNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNNPLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 179 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILVSHNVYITADKQKNGIKANFKIRHNVEDGSVQLADHYQQNTPIGDGPVLLPDNHYLSYQSVLSKDPNEKRDHMVLLEFVTAAGITLGMDELYKGGTGGSMSKGEELFTGVVPILVELDGDVNGHKFSVRGEGEGDATNGKLTLKLICTTGKLPVPWPTLVTTLGYGVQCFARYPDHMKQHDFFKSAMPEGYVQERTISFKDDGTYKTRAEVKFEGDTLVNRIELKGIDFKEDGNILGHKLEYNFNNPLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 180 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITAQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDSLLAGERAKAKKPDNWEIVGKPQSQEAYGCMLRKDDPQFKKLMDDTIAQVQTSGEAEKWFDKWFKNPILVSHNVYITADKQKNGIKANFKIRHNVEDGSVQLADHYQQNTPIGDGPVLLPDNHYLSYQSVLSKDPNEKRDHMVLLEFVTAAGITLGMDELYKGGTGGSMSKGEELFTGVVPILVELDGDVNGHKFSVRGEGEGDATNGKLTLKLICTTGKLPVPWPTLVTTLGYGVQCFARYPDHMKQHDFFKSAMPEGYVQERTISFKDDGTYKTRAEVKFEGDTLVNRIELKGIDFKEDGNILGHKLEYNFNNPLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 181 - protein, 606 aa, synthetic construct; identical to SEQ ID NO: 179

SEQ ID NO: 182 - protein, 606 aa, synthetic construct
METDTLLLWVLLLWVPGSTGDRSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILGYHNIYITADKQKNGIKANFKIRHNVEDGSVQLADHYQQNTPIGDGPVLLPDNHYLSTQSVLSKDPNEKRDHMVLLEFRTAAGITLGMDELYKGGTGGSMSKGEELFTGVVPILVELDGDVNGHKFSVRGEGEGDATNGKLTLKFICTTGKLPVPWPTLVTTLSHGVQCFSRYPDHMKQHDFFKSAMPEGYVQERTISFKDDGTYKTRAEVKFEGDTLVNRIELKGIDFKEDGNILGHKLEYNFNEQLNMNFELSDEMKALFKEPNDKALKLQVDEQKLISEEDLNAVGQDTQEVIVVPHSLPFKVVVISAILALVVLTIISLIILIMLWQKKPR

SEQ ID NO: 183 - protein, 728 aa, synthetic construct
METDTLLLWVLLLWVPGSTGDRSKLEIFSWWAGDEGPALEALIRLYKQKYPGVEVINATVTGGAGVNARAVLKTRMLGGDPPDTFQVAAGMELIGTWVVANRMEDLSALFRQEGWLQAFPKGLIDLISYKGGIWSVPVNIHRSNVMWYLPAKLKEWGVNPPRTWDEFLATCQTLKQKGLEAPLALGENWTQQHLWESVALAVLGPDDWNNLWNGKLKFTDPKAVRAWEVFGRVLDCANKDAAGLSWQQAVDRVVQGKAAFNVMGDWAAGYMTTTLKLKPGTDFAWAPSPGTQGVFMMLSDSFGLPKGAKNRQNAINWLRLVGSKEGQDTFNPLKGSIAARLDSDPSKYPASHNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNNPNAYGQSAMRDWRSNRIVGSLVAGAVAPESFMSQFGTVMEIFLQTRNPQAAANAAQAIADQVGLGRLGQLQVDEQKLISEEDLNAVGQDTQEVIVVPHSLPFKVVVISAILALVVLTIISLIILIMLWQKKPR

SEQ ID NO: 184 - protein, 650 aa, synthetic construct
METDTLLLWVLLLWVPGSTGDRSESINFVSWGGSTQDAQKQAWADPFSKASGITVVQDGPTDYGKLKAMVESGNVQWDVVDVEADFALRAAAEGLLEPLDFSVIQRDKIDPRFVSDHGVGSFLFSFVLGYNEGKLGASKPQDWTALFDTKTYPGKRALYKWPSPGVLELALLADGVPADKLYPLDLDRAFKKLDTIKKDIVWWGGGAQSQQLLASGEVSMGQFWNGRIHALQEDGAPVGVSWKQNLVMADILVVPKGTKNKAAAMKFLASASSAKGQDDFSALTAYAPVNIDSVQRLDLAQVRITADKQKNGIMANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNWNANLAPNLPTAYVKDQITLDFAYWAKNGPAIATRWNEWLVKLQVDLQVDEQKLISEEDLNAVGQDTQEVIVVPHSLPFKVVVISAILALVVLTIISLIILIMLWQKKPR

SEQ ID NO: 185 - protein, 560 aa, synthetic construct
MHHHHHHGYPYDVPDYAGAQPARSANDTVVVGSIIFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLILQVDEQKLISEEDLN

SEQ ID NO: 186 - protein, 608 aa, synthetic construct
METDTLLLWVLLLWVPGSTGDRSANDTVVVGSIIFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLILQVDEQKLISEEDLNAVGQDTQEVIVVPHSLPFKVVVISAILALVVLTIISLIILIMLWQKKPR

SEQ ID NO: 187 - protein, 8 aa, synthetic construct
PILVSHNV

SEQ ID NO: 188 - protein, 8 aa, synthetic construct
PILGYHNV

SEQ ID NO: 189 - protein, 8 aa, synthetic construct
YNFNNPLN

SEQ ID NO: 190 - protein, 8 aa, synthetic construct
YNFNEQLN

SEQ ID NO: 191 - protein, 5 aa, synthetic construct
SHNVY

SEQ ID NO: 192 - protein, 5 aa, synthetic construct
LAQVR

SEQ ID NO: 193 - protein, 5 aa, synthetic construct
SFGFP

SEQ ID NO: 194 - protein, 5 aa, synthetic construct
SVLAP

SEQ ID NO: 195 - protein, 5 aa, synthetic construct
ANLAP

SEQ ID NO: 196 - protein, 7 aa, synthetic construct
  VARIANT 2: note = any amino acid
PXSHNVY

SEQ ID NO: 197 - protein, 6 aa, synthetic construct
HHHHHH

(19) United States
(12) Patent Application Publication
(10) Pub. No.: US 2024/0085411 A1
(43) Pub. Date: Mar. 14, 2024

(54) GENETICALLY ENCODED BIOSENSORS

(71) Applicant: Howard Hughes Medical Institute, Chevy Chase, MD (US)

(72) Inventors: Jonathan S. Marvin, Arlington, VA (US); Loren Looger, Sterling, VA (US)

(21) Appl. No.: 18/322,480

(22) Filed: May 23, 2023

Related U.S. Application Data
(63) Continuation of application No. 17/515,289, filed on Oct. 29, 2021, now Pat. No. 11,698,374.
Application No. 17/515,289 is a division of application No. 16/902,160, filed on Jun. 15, 2020, now Pat. No. 11,162,942.
Application No. 16/902,160 is a continuation of application No. 16/002,697, filed on Jun. 7, 2018, now Pat. No. 10,684,282.
Application No. 16/002,697 is a continuation-in-part of application No. 15/904,574, filed on Feb. 26, 2018, now Pat. No. 10,060,920.
Application No. 15/904,574 is a division of application No. 15/664,326, filed on Jul. 31, 2017, now Pat. No. 9,939,437.
Application No. 15/664,326 is a division of application No. 14/350,199, filed on Nov. 18, 2014, now Pat. No. 9,719,992.
(60) Provisional application No. 61/544,867, filed on Oct. 7, 2011.

(51) Int. Cl. G01N 33/557; C07K 14/195; C07K 14/245; C07K 14/435; G01N 33/58; G01N 33/68

(52) U.S. Cl. CPC G01N 33/557; C07K 14/195; C07K 14/245; C07K 14/43595; G01N 33/582; G01N 33/68; G01N 33/6812; C07K 2319/20; C07K 2319/24; C07K 2319/60; G01N 2400/00; G01N 2458/00

(57) ABSTRACT

The present disclosure provides, inter alia, genetically encoded recombinant peptide biosensors comprising analyte-binding framework portions and signaling portions, wherein the signaling portions are present within the framework portions at sites or amino acid positions that undergo a conformational change upon interaction of the framework portion with an analyte.

### CROSS REFERENCE TO RELATED APPLICATIONS

This application is a Continuation of, and claims the benefit of priority under 35 U.S.C. § 121 to, U.S. application Ser. No. 17/515,289 filed Oct. 29, 2021, now allowed, which is a Divisional of, and claims the benefit of priority under 35 U.S.C. § 121 to, U.S. application Ser. No. 16/902,160 filed Jun. 15, 2020, which is a Continuation of, and claims priority under 35 U.S.C. § 121 to, U.S. application Ser. No. 16/002,697 filed Jun. 7, 2018, which is a Continuation-In-Part of, and claims priority under 35 U.S.C. § 120 to, U.S. application Ser. No. 15/904,574 filed Feb. 26, 2018, which is a Divisional application of, and claims the benefit of priority under 35 U.S.C. § 121 to, U.S. application Ser. No. 15/664,326 filed Jul. 31, 2017, which is a Divisional application of, and claims the benefit of priority under 35 U.S.C. § 121 to, U.S. application Ser. No. 14/350,199 filed Nov. 18, 2014, which is a U.S. National Phase application of, and claims the benefit of priority under 35 U.S.C. 371 to, International Application No. PCT/US2012/059219 filed Oct. 8, 2012, which claims the benefit of priority under 35 U.S.C. § 119(e) to U.S. Application No. 61/544,867 filed Oct. 7, 2011, all of which are incorporated by reference in their entirety.

### SEQUENCE LISTING

This application contains a Sequence Listing that has been submitted electronically as an XML file named “30872-0020004_SL_ST26.XML.” The XML file, created on May 23, 2023, is 288,585 bytes in size. The material in the XML file is hereby incorporated by reference in its entirety.

### TECHNICAL FIELD

This disclosure relates to genetically encoded biosensors and methods for the design, production, and use of such biosensors.

### BACKGROUND

Protein-based sensors that transduce microscopic binding events into macroscopically observable signals are available to allow real-time visualization of a variety of biological events and/or molecules (Frommer et al., Chem. Soc. Rev., 38:2833-2841, 2009). Such sensors can be targeted and/or expressed in living cells, tissues, and organisms, and permit imaging with minimally invasive techniques (Okumoto, Curr. Opin. Biotechnol., 21:45-54, 2010). Application of these sensors is limited by the narrow range of analytes that can be detected and/or by their inability to distinguish signal over noise.

### SUMMARY

In one aspect, a recombinant peptide biosensor is provided that includes an analyte-binding framework portion and a signaling portion, wherein the signaling portion is present within the framework portion at a site or amino acid position that undergoes a conformational change upon interaction of the framework portion with a defined, specific, or selected analyte.

In one embodiment, the signaling portion is allosterically regulated by the framework portion such that signaling from the signaling portion is altered upon interaction of the framework portion with the analyte. In some embodiments, signaling by the signaling portion detectably increases upon interaction of the framework portion with the analyte. In some embodiments, signaling by the signaling portion detectably decreases upon interaction of the framework portion with the analyte. In some embodiments, signaling by the signaling portion is proportional to the level of interaction between the framework portion and the analyte.

In some embodiments, the signaling portion is a superfolder (SF) fluorescent protein (see, for example, Pedelacq et al., 2006, Nature Biotechnol., 24:79-88), a protein that exhibits robust folding, even when fused to a protein that folds poorly. In some embodiments, the SF protein is circularly permuted. In some embodiments, the SF protein is a green fluorescent protein, a yellow fluorescent protein, a red fluorescent protein, or a blue fluorescent protein.

In some embodiments, the framework portion has a first structure in the absence of an analyte and a second structure, that is detectably distinct from the first structure, in the presence of the analyte. In some embodiments, the conformational change between the first structure and the second structure allosterically regulates the signaling portion. In some embodiments, the framework portion is a periplasmic binding protein (PBP) or a variant of a PBP.

In some embodiments, the analyte-binding framework portion binds specifically to an analyte selected from the group consisting of glucose, maltose, phosphonate, glutamate, GABA, and ACh.

In another aspect, a recombinant peptide biosensor is provided that includes an amino acid sequence having at least 90% identity to a recombinant peptide biosensor selected from the group consisting of SEQ ID NOs: 176-182, wherein the recombinant peptide biosensor binds specifically to glutamate.

In one embodiment, the recombinant peptide biosensor includes a recombinant peptide biosensor selected from the group consisting of SEQ ID NOs: 176-182 comprising 10 or fewer conservative amino acid substitutions, wherein the recombinant peptide biosensor binds specifically to glutamate. In some embodiments, the recombinant peptide biosensor includes a recombinant peptide biosensor selected from the group consisting of SEQ ID NOs: 176-182.

In still another aspect, a recombinant peptide biosensor is provided that includes an amino acid sequence having at least 90% identity to a recombinant peptide biosensor having the sequence shown in SEQ ID NO: 183, wherein the recombinant peptide biosensor binds specifically to glucose.

In some embodiments, the recombinant peptide biosensor includes a recombinant peptide biosensor having the sequence shown in SEQ ID NO: 183 comprising 10 or fewer conservative amino acid substitutions, wherein the recombinant peptide biosensor binds specifically to glucose. In some embodiments, the recombinant peptide biosensor includes a recombinant peptide biosensor having the sequence shown in SEQ ID NO: 183.

In one aspect, a recombinant peptide biosensor is provided that includes an amino acid sequence having at least 90% identity to a recombinant peptide biosensor having the sequence shown in SEQ ID NO: 184, wherein the recombinant peptide biosensor binds specifically to GABA.

In one embodiment, the recombinant peptide biosensor includes a recombinant peptide biosensor having the sequence shown in SEQ ID NO: 184 comprising 10 or fewer conservative amino acid substitutions, wherein the recombinant peptide biosensor binds specifically to GABA. In one embodiment, the recombinant peptide biosensor includes a recombinant peptide biosensor having the sequence shown in SEQ ID NO: 184.

In another aspect, a recombinant peptide biosensor is provided that includes an amino acid sequence having at least 90% identity to a recombinant peptide biosensor having a sequence selected from the group consisting of SEQ ID NO: 185 and 186, wherein the recombinant peptide biosensor binds specifically to ACh.

In one embodiment, the recombinant peptide biosensor includes a recombinant peptide biosensor having a sequence selected from the group consisting of SEQ ID NO: 185 and 186 comprising 10 or fewer conservative amino acid substitutions, wherein the recombinant peptide biosensor binds specifically to ACh. In one embodiment, the recombinant peptide biosensor includes a recombinant peptide biosensor having a sequence selected from the group consisting of SEQ ID NO: 185 and 186.

In one aspect, a nucleic acid is provided that encodes a recombinant peptide biosensor as described herein.

In one aspect, a vector is provided that includes a nucleic acid as described herein.

In one aspect, a cell is provided that includes a nucleic acid as described herein.

In one aspect, a cell is provided that includes a vector as described herein.

In one aspect, a kit is provided that includes a recombinant peptide biosensor as described herein, a nucleic acid as described herein, a vector as described herein, and/or the cell as described herein.

In still another aspect, a method is provided for detecting glutamate, the method comprising detecting a level of fluorescence emitted by a recombinant peptide biosensor, the peptide biosensor having an amino acid sequence selected from the group consisting of SEQ ID NOs: 176-182, and correlating the level of fluorescence with the presence of glutamate.

In some embodiments, the recombinant peptide biosensor is expressed from a nucleic acid. In some embodiments, the method includes contacting the recombinant peptide biosensor with a sample comprising glutamate. In some embodiments, the method includes correlating the level of fluorescence with a concentration of glutamate. In some embodiments, the method includes comparing the level of fluorescence with a level of fluorescence emitted by the recombinant peptide biosensor in the presence of a sample comprising a known concentration or range of concentrations of glutamate. In some embodiments, the method is performed in vitro.

In some aspects, a method for detecting glucose is provided, the method comprising detecting a level of fluorescence emitted by a recombinant peptide biosensor, the peptide biosensor having an amino acid sequence shown in SEQ ID NO: 183, and correlating the level of fluorescence with the presence of glucose.

In some aspects, a method for detecting GABA is provided, the method comprising detecting a level of fluorescence emitted by a recombinant peptide biosensor, the peptide biosensor having an amino acid sequence shown in SEQ ID NO: 184, and correlating the level of fluorescence with the presence of GABA.

In some aspects, a method for detecting ACh is provided, the method comprising detecting a level of fluorescence emitted by a recombinant peptide biosensor, the peptide biosensor having an amino acid sequence selected from the group consisting of SEQ ID NOs: 185 and 186, and correlating the level of fluorescence with the presence of ACh.

In some aspects, a method for detecting a defined, selected, or specific analyte is provided, the method comprising detecting a level of fluorescence emitted by a recombinant peptide biosensor of claim 1; and correlating the level of fluorescence with the presence of a defined, selected, or specific analyte

In some embodiments, the recombinant peptide biosensor is expressed from a nucleic acid. In some embodiments, the method includes contacting the recombinant peptide biosensor with a sample comprising the analyte. In some embodiments, the method includes correlating the level of fluorescence with a concentration of the analyte. In some embodiments, the method includes comparing the level of fluorescence with a level of fluorescence emitted by the recombinant peptide biosensor in the presence of a sample comprising a known concentration or range of concentrations of the analyte. In some embodiments, the method is performed in vitro. In some embodiments, the analyte is selected from the group consisting of glutamate, glucose, GABA, and ACh.

The present disclosure provides genetically encoded recombinant peptides containing an analyte-binding framework portion linked (e.g., operably linked) to a signaling portion, wherein the signaling portion is allosterically regulated by the framework portion upon interaction of the framework portion with an analyte (e.g., a defined, selected, and/or specific analyte). These constructs can be used as biosensors, e.g., to transduce microscopic binding events into macroscopically observable signals.

The present disclosure provides, in part, recombinant peptides for use as biosensors (e.g., recombinant peptide biosensors) that include (e.g., comprise, consist essentially of, or consist of), e.g., include at least, an analyte-binding framework portion and a signaling portion. As described in further detail herein, such signaling portions are present within the framework portion at a site or amino acid position that undergoes a conformational change (e.g., a conformational change sufficient to alter a physical and/or functional characteristic of the signaling portion, e.g., a substantial conformational change) upon interaction of the framework portion with a defined, specific, or selected analyte (e.g. such as an analyte to which the framework portion or a region thereof, and/or the biosensor, specifically binds).

For example, in some instances, the signaling portion is allosterically regulated by the framework portion such that signaling from the signaling portion is altered (e.g. wherein a first level of signaling is altered or changed to a second level of signaling that can be distinguished using routine methods of detection from the first) upon interaction of the framework portion with the analyte. In some instances, signaling by the signaling portion can detectably increase or decrease upon interaction of the framework portion with the analyte. In some instances, signaling by the signaling portion upon interaction of the biosensor with a defined, specific, or selected analyte (e.g. such as an analyte to which the framework portion or a region thereof, and/or the biosensor, specifically binds) can be proportional or can correlate with to the level of interaction between the framework portion and the analyte such that the level of interaction can be determined from the signaling or alteration thereof.

In some instances, framework portions of the biosensors disclosed herein have a first structure in the absence of an analyte and a second structure that is detectably distinct from the first structure in the presence of the analyte. In some instances, the conformational change between the first structure and the second structure allosterically regulates the signaling portion.

In some instances, framework portions of the biosensors disclosed herein can be, or can include (e.g., comprise, consist essentially of, or consist of), periplasmic binding proteins (PBP) or variants of a PBP. In some instances, exemplary PBPs or variants thereof can include, but are not limited to, peptides with at least 90% identity to a peptide selected from the group consisting of SEQ ID NO:105, SEQ ID NO: 106, SEQ ID NO:107, SEQ ID NO:108, SEQ ID NO:109, SEQ ID NO: 110, SEQ ID NO:111, SEQ ID NO:113, and SEQ ID NO:114. In some instances, exemplary PBPs or variants thereof can include, but are not limited to, peptides with at least 95% identity to a peptide selected from the group consisting of SEQ ID NO:105, SEQ ID NO: 106, SEQ ID NO:107, SEQ ID NO:108, SEQ ID NO:109, SEQ ID NO: 110, SEQ ID NO:111, SEQ ID NO:113, and SEQ ID NO:114. In some instances, exemplary PBPs or variants thereof can include, but are not limited to, peptides selected from the group consisting of SEQ ID NO:105, SEQ ID NO: 106, SEQ ID NO:107, SEQ ID NO:108, SEQ ID NO:109, SEQ ID NO: 110, SEQ ID NO:111, SEQ ID NO:113, and SEQ ID NO:114. In some instances, exemplary PBPs or variants thereof can include, but are not limited to, peptides selected from the group consisting of SEQ ID NO:105, SEQ ID NO: 106, SEQ ID NO:107, SEQ ID NO:108, SEQ ID NO:109, SEQ ID NO: 110, SEQ ID NO:111, SEQ ID NO:113, and SEQ ID NO:114 comprising 10 or fewer conservative amino acid substitutions. PBPs or variants thereof disclosed herein can be truncated.

In some instances, signaling portions of the biosensors disclosed herein can be or can include (e.g., comprise, consist essentially of, or consist of) one or more (e.g., one, two three, four, five, and less than ten) circularly permuted fluorescent proteins (cpFPs). Such cpFPs can be include but are not limited to, for example, green fluorescent proteins, yellow fluorescent proteins, red fluorescent proteins, and/or blue fluorescent proteins.

In some instances, biosensors disclosed herein, e.g., analyte-binding framework portions of biosensors disclosed herein, can bind (e.g., bind specifically) to glucose. Such sensors can be referred to as glucose binding biosensors or glucose biosensors.

In some instances, biosensors disclosed herein, e.g., analyte-binding framework portions of biosensors disclosed herein, can bind (e.g., bind specifically) to maltose. Such sensors can be referred to as maltose binding biosensors or maltose biosensors.

In some instances, biosensors disclosed herein, e.g., analyte-binding framework portions of biosensors disclosed herein, can bind (e.g., bind specifically) to phosphonate. Such sensors can be referred to as phosphonate binding biosensors or phosphonate biosensors.

In some instances, biosensors disclosed herein, e.g., analyte-binding framework portions of biosensors disclosed herein, can bind (e.g., bind specifically) to glutamate. Such sensors can be referred to as glutamate binding biosensors or glutamate biosensors.

In some instances, biosensors disclosed herein can include (e.g., comprise, consist essentially of, or consist of): an amino acid sequence with at least 90% identity to a recombinant peptide biosensor selected from the group consisting of SEQ ID NO: 1, 2, 3, 4, 5, 6, 7, 8, 50, 51, 52, and 53, wherein the recombinant peptide biosensor binds specifically to maltose; a recombinant peptide biosensor selected from the group consisting of SEQ ID NO: 1, 2, 3, 4, 5, 6, 7, 8, 50, 51, 52, and 53 comprising 10 or fewer conservative amino acid substitutions, wherein the recombinant peptide biosensor binds specifically to maltose; and/or a recombinant peptide biosensor selected from the group consisting of SEQ ID NO: 1, 2, 3, 4, 5, 6, 7, 8, 50, 51, 52, and 53.

In some instances, biosensors disclosed herein can include (e.g., comprise, consist essentially of, or consist of): an amino acid sequence with at least 90% identity to a recombinant peptide biosensor selected from the group consisting of SEQ ID NO: 62 and 63, wherein the recombinant peptide biosensor binds specifically to glutamate; a recombinant peptide biosensor selected from the group consisting of SEQ ID NO: 62 and 63 comprising 10 or fewer conservative amino acid substitutions, wherein the recombinant peptide biosensor binds specifically to glutamate; and/or a recombinant peptide biosensor selected from the group consisting of SEQ ID NO: 62 and 63.

In some instances, biosensors disclosed herein can include (e.g., comprise, consist essentially of, or consist of): an amino acid sequence with at least 90% identity to a recombinant peptide biosensor selected from the group consisting of SEQ ID NO: 77 and 78, wherein the recombinant peptide biosensor binds specifically to phosphonate; a recombinant peptide biosensor selected from the group consisting of SEQ ID NO: 77 and 78 comprising 10 or fewer conservative amino acid substitutions, wherein the recombinant peptide biosensor binds specifically to phosphonate; and/or a recombinant peptide biosensor selected from the group consisting of SEQ ID NO: 77 and 78.

In some instances, biosensors disclosed herein can include (e.g., comprise, consist essentially of, or consist of): an amino acid sequence with at least 90% identity to a recombinant peptide biosensor selected from the group consisting of SEQ ID NO: 91, 92, 93 and 94, wherein the recombinant peptide biosensor binds specifically to glucose; a recombinant peptide biosensor selected from the group consisting of SEQ ID NO: 91, 92, 93 and 94 comprising 10 or fewer conservative amino acid substitutions, wherein the recombinant peptide biosensor binds specifically to glucose; and/or a recombinant peptide biosensor selected from the group consisting of SEQ ID NO: 91, 92, 93 and 94.

In some instances, biosensors disclosed herein can include (e.g., comprise, consist essentially of, or consist of): SEQ ID NO:91; SEQ ID NO:92; SEQ ID NO:93; SEQ ID NO:95.

In some instances, any recombinant biosensor disclosed herein can be isolated and/or purified. The terms “isolated” or “purified,” when applied to a biosensor disclosed herein includes nucleic acid proteins and peptides that are substantially free or free of other cellular material or culture medium when produced by recombinant techniques, or substantially free or free of precursors or other chemicals when chemically synthesized.

The disclosure also provides, in part, nucleic acids (e.g., isolated and/or purified nucleic acids) encoding any one or more of the recombinant peptide biosensors disclosed herein. For example, nucleic acids can encode: an amino acid sequence with at least 90% identity to a recombinant peptide biosensor selected from the group consisting of SEQ ID NO: 1, 2, 3, 4, 5, 6, 7, 8, 50, 51, 52, and 53, wherein the recombinant peptide biosensor binds specifically to maltose; a recombinant peptide biosensor selected from the group consisting of SEQ ID NO: 1, 2, 3, 4, 5, 6, 7, 8, 50, 51, 52, and 53 comprising 10 or fewer conservative amino acid substitutions, wherein the recombinant peptide biosensor binds specifically to maltose; a recombinant peptide biosensor selected from the group consisting of SEQ ID NO: 1, 2, 3, 4, 5, 6, 7, 8, 50, 51, 52, and 53; an amino acid sequence with at least 90% identity to a recombinant peptide biosensor selected from the group consisting of SEQ ID NO: 62 and 63, wherein the recombinant peptide biosensor binds specifically to glutamate; a recombinant peptide biosensor selected from the group consisting of SEQ ID NO: 62 and 63 comprising 10 or fewer conservative amino acid substitutions, wherein the recombinant peptide biosensor binds specifically to glutamate; a recombinant peptide biosensor selected from the group consisting of SEQ ID NO: 62 and 63; an amino acid sequence with at least 90% identity to a recombinant peptide biosensor selected from the group consisting of SEQ ID NO: 77 and 78, wherein the recombinant peptide biosensor binds specifically to phosphonate; a recombinant peptide biosensor selected from the group consisting of SEQ ID NO: 77 and 78 comprising 10 or fewer conservative amino acid substitutions, wherein the recombinant peptide biosensor binds specifically to phosphonate; a recombinant peptide biosensor selected from the group consisting of SEQ ID NO: 77 and 78; an amino acid sequence with at least 90% identity to a recombinant peptide biosensor selected from the group consisting of SEQ ID NO: 91, 92, 93 and 94, wherein the recombinant peptide biosensor binds specifically to glucose; a recombinant peptide biosensor selected from the group consisting of SEQ ID NO: 91, 92, 93 and 94 comprising 10 or fewer conservative amino acid substitutions, wherein the recombinant peptide biosensor binds specifically to glucose; a recombinant peptide biosensor selected from the group consisting of SEQ ID NO: 91, 92, 93 and 94; and/or SEQ ID NO:91; SEQ ID NO:92; SEQ ID NO:93; SEQ ID NO:95.

In some instances, the disclosure includes vectors containing one or a plurality of the nucleic acids disclosed herein and cells containing such vectors. In some instances, the disclosure provides cells containing one or a plurality of nucleic acids disclosed herein.

In some instances, the disclosure includes kits related to the biosensors and nucleic acids disclosed herein Such kits can include or contain, for example, a biosensor, a nucleic acid encoding a biosensor, vectors, and/or cells, provided herein.

In some instances, the disclosure provides methods related to the biosensors and nucleic acids disclosed herein. Such methods can include methods of making, using, and/or selling the biosensors and nucleic acids disclosed herein. For example, methods can include methods for producing genetically encoded recombinant peptide biosensors. In such instances, methods can include, for example, selecting a framework portion that binds specifically to a target analyte and that undergoes a conformational change upon interacting binding to the target analyte, identifying a site or amino acid position within the selected framework portion where or around which the conformational change occurs, and inserting a signaling portion into the site or amino acid position. In some instances, framework portions include periplasmic binding proteins (PBPs) disclosed herein. Exemplary PBPs include PBPs that bind (e.g., bind specifically) to glucose.

In some instances, the present disclosure includes methods for detecting glucose, e.g., in a sample containing a level of glucose. Such methods can include, detecting a level of fluorescence emitted by a recombinant peptide biosensor, the peptide biosensor having an amino acid sequence selected from the group consisting of SEQ ID NO: 91, 92, 93 and 94, and correlating the level of fluorescence with the presence of glucose. In some instances, recombinant peptide biosensors used in the methods herein are expressed from nucleic acids. In some instances, methods include contacting the recombinant peptide biosensor with a test sample (e.g., a sample comprising glucose). In some instances, methods can include the level of fluorescence emitted by a biosensor (e.g., a biosensor bound to glucose) with a concentration glucose in the sample. Such correlation can include, for example, comparing the level of fluorescence with a level of fluorescence emitted by the recombinant peptide biosensor in the presence of a sample comprising a known concentration or range of concentrations of glucose. In some instance, the level of fluorescence emitted by the recombinant peptide biosensor in the presence (e.g., bound or bound specifically to) of a sample comprising a known concentration or range of concentrations of glucose is stored on an electronic database.

One of skill will appreciate that such methods can be adapted for any defined, specific, or selected analyte. For example, in some instances, the disclosure provides methods for detecting a defined, selected, or specific analyte. These methods can include detecting a level of fluorescence emitted by a recombinant peptide biosensor expressed from a nucleic acid and correlating the level of fluorescence with the presence the defined, selected, or specific analyte. In some instances, methods include contacting the recombinant peptide biosensor with a sample comprising the analyte. In some instances, methods include correlating the level of fluorescence with a concentration of the analyte. In some instances, methods include comparing the level of fluorescence with a level of fluorescence emitted by the recombinant peptide biosensor in the presence of a sample comprising a known concentration or range of concentrations of the analyte, wherein the level of fluorescence emitted by the recombinant peptide biosensor in the presence of a sample comprising a known concentration or range of concentrations of the analyte is stored on an electronic database.

In some instances, the present disclosure provides methods for detecting a defined, selected, or specific analyte, the method comprising detecting a level of fluorescence emitted by a recombinant peptide biosensor as described herein; and correlating the level of fluorescence with the presence of a defined, selected, or specific analyte. In some instances, recombinant peptide biosensors can be expressed from a nucleic acid. In some instances, methods can include contacting the recombinant peptide biosensor with a sample comprising the analyte. In some instances, methods can include correlating the level of fluorescence with a concentration of the analyte and, optionally, comparing the level of fluorescence with a level of fluorescence emitted by the recombinant peptide biosensor in the presence of a sample comprising a known concentration or range of concentrations of the analyte. In some instances, the level of fluorescence emitted by the recombinant peptide biosensor in the presence of a sample comprising a known concentration or range of concentrations of the analyte is stored on an electronic database.

Methods herein can be performed in vitro.

In some instances, the present disclosure provides compositions containing any one or a plurality of the peptide biosensors and/or nucleic acids disclosed herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1|Cartoon representation showing ligand bound Escherichia coli malto-dextrin-binding protein (EcMBP) and potential circularly-permuted fluorescent protein (cpFP) insertion sites.

FIG. 2|Cartoon representation showing ligand bound Pyrococcus furiosus maltotriose binding protein (PfMBP) and potential cpFP insertion sites.

FIG. 3|Cartoon representation showing ligand bound E. coli glutamate-binding protein (EcYbeJ) and potential cpFP insertion sites.

FIG. 4|Cartoon representation showing ligand bound E. coli phosphonate-binding protein (EcPhnD) and potential cpFP insertion sites.

FIG. 5|Cartoon representation showing ligand bound Thermus thermophilus glucose binding protein (TtGBP) and potential cpFP insertion sites.

FIG. 6A-B|Changes in EcMBP upon maltose binding and locations at which circularly-permuted fluorescent protein (cpFP) was inserted are shown as colored spheres at the Ca positions. Yellow: 165-166, Green: 175-176, Cyan: 311-312, Violet: 317-318 (FIG. 6A). (FIG. 6B) shows backbone structural changes. The Ca dihedral is calculated from the four atoms: Cαi+2, Cαi+1, Cαi, Cαi-1. ΔDihedral is calculated as the difference in dihedrals between the closed (1ANF) and open (1OMP) states of MBP, and corrected to fall within a range of −180° to 180°. The regions near residues 175 and 311 are labeled. There is a crystallographic artifact at the N-terminus resulting in the appearance of significant structural changes.

FIG. 7A|Amino acid sequence of MBP-165-cpGFP (SEQ ID NO:1).

FIG. 7B|Amino acid sequence of MBP-165-cpGFP.PPYF (SEQ ID NO:2).

FIG. 7C|Amino acid sequence of MBP-165-cpGFP.PCF (SEQ ID NO:3).

FIG. 8A|Amino acid sequence of MBP-175-cpGFP (SEQ ID NO:4).

FIG. 8B|Amino acid sequence of MBP-175-cpGFP.L1-HL (SEQ ID NO:5).

FIG. 9A|Amino acid sequence of MBP-311-cpGFP (SEQ ID NO:6).

FIG. 9B|Amino acid sequence of MBP-311-cpGFP.L2-NP (SEQ ID NO:7).

FIG. 10|Amino acid sequence of MBP-317-cpGFP (SEQ ID NO:8).

FIGS. 11A-11D|Line charts showing EcMBP plot of ΔF/F for clarified lysate screen of cpGFP linker-screens at insertion points 165, 175, 311, and 317. The horizontal dashed line at zero indicates no fluorescence change. Standard deviations in ΔF/F are less than 10% of an average ΔF (repetitions for MBP165-cpGFP.PPYF yields ΔF/F values of 2.51, 2.63, and 2.54).

FIG. 12|Isothermal titration calorimetry (ITC) of MBP317-cpGFP with maltose.

FIG. 13|Graph showing EcMBP165-cpGFP.PPYF affinity variant binding maltose-binding curves. Binding curves for affinity variants of MBP165-cpGFP.PPYF. Data is fit to a single-binding site isotherm. Curve-fit affinities are: WT binding pocket, 5 μM (●); W230A, 32 μM (▪); W62A, 375 μM (▴); W340A, >1 mM (▾); 1329W, 11 μM (□).

FIGS. 14A-14D|Line graphs showing maltose and sucrose binding curves for wild-type and 5-7 variants of the EcMBP-cpGFP sensors. Maltose (black) and sucrose (red) binding curves for wild-type (filled, solid lines) and 5-7 variants (open, dashed lines) of the MBP-cpGFP sensors. MBP165-cpGFP.PPYF (FIG. 14A); MBP165-cpGFP.PCF (FIG. 14B); MBP175-cpGFP.L1-HL (FIG. 14C); MBP311-cpGFP.L2-NP (FIG. 14D).

FIGS. 15A-15D|Line graphs showing emission spectra for colored variants of EcMBP sensors. Fluorescence emission spectra of the MBP165-Blue, Cyan, Green, and Yellow wild-type sensors (FIG. 15A) and the 5-7 variants (FIG. 15B) in the absence of ligand (dashed lines, open circles), with 10 mM maltose (solid lines, filled circles), or 10 mM sucrose (solid lines, filed squares). Sensors were excited at 383, 433, 485, and 485 nm, respectively. Titration of maltose and sucrose in the Blue, Cyan, Green, and Yellow MBP165 wild-type sensors (FIG. 15C) and for the 5-7 variants (FIG. 15D). Filled circles are titration of maltose, open circles are titration of sucrose. For the wild-type sensors, Kds for maltose binding are: Blue 3.3 μM, Cyan 13 Green 4.5 μM, Yellow 3.3 μM. No sucrose binding is observed. For the 5-7 variants, Kd of Green is 2.4 mM (sucrose) and 7.1 mM (maltose). Kd of Yellow is 2.5 mM (sucrose) and 4.5 mM (maltose).

FIG. 16|Plot of ΔF/F for clarified lysate screen of MBP165-cpBFP linker-screen. The horizontal dashed line at zero indicates no fluorescence change.

FIGS. 17A-17B|Line graphs showing maltose binding. Blue (wt binding pocket) has an affinity of 2.7 μM. Green (W230A) has an affinity of 40 μM. Yellow (W62A) has an affinity of 350 μM. Cyan (W340A) has an affinity of approximately 1.7 mM. Data is plotted at ΔF/F (FIG. 17A) or normalized to Fractional Saturation (FIG. 17B).

FIGS. 18A-18C|Images bacterial cells expressing (FIG. 18A) EGFP, (FIG. 18B) PPYF, or (FIG. 18C) PPYF.T203V in the absence (top) and presence (bottom) of maltose.

FIGS. 19A-19B|Line graphs showing EcMBP-cpGFP.PPYF.T203V 2-photon excitation spectra. MBP165-cpAzurite.L2-FE (FIG. 19A), -cpCFP.PCF (FIG. 19A), -cpGFP.PPYF (FIG. 19B), and -cpYFP.PPYF (FIG. 19B) were excited at the wavelengths indicated and emission measured through appropriate wavelength filters. Two graphs are shown to present different y-axis scales. Optimal ΔF/F values for 2-photon excitation of the spectral variants of MBP165 are: -cpAzurite, 1.1 (ex 760 nm); -cpCFP, 2.3 (ex 830-960 nm); -cpGFP, 10.0 (ex 940 nm); -cpYFP, 2.6 (ex 940 nm).

FIGS. 20A-20C|Images showing EcMBP-cpGFP.PPYF.T203V expressing HEK cells. Images of individual HEK293 cells expressing membrane displayed PPYF.T203V in the absence of maltose (FIG. 20A), in the presence of 1 mM maltose (FIG. 20B), and after washout with maltose-free buffer (FIG. 20C). Scale bars are 10 μm.

FIGS. 21A-21B|Graphs showing quantification of fluorescence of EcMBP-cpGFP.PPYF.T203V when displayed on the surface of HEK cells. (FIG. 21A) Concentration dependence. (FIG. 21B) Observed fluorescence after a “puff” of HBSS solution containing 1 mM maltose and 2.5 nM Alexa Fluor® 568 (Invitrogen, Carlsbad, CA).

FIGS. 22A-22D|Cartoon representations and close-up views of inter-domain linkers and selected amino acids of the cpGFP chromophore environment of the structure of MBP175-cpGFP.L1-HL (FIG. 22A and FIG. 22B) and MBP311-cpGFP.L2-NP (FIG. 22C and FIG. 22D) bound to maltose. The MBP domain is colored as in FIG. 1. The cpGFP domain is green and the inter-domain linkers are colored white. The cpGFP chromophore is displayed as sticks and the bound maltose as red and white spheres. Ordered water molecules are represented as red spheres. Selected hydrogen bonds are displayed as dashed black lines. β-strands 10 and 11 of cpGFP are displayed as semi-transparent for clarity. The 2Fo-Fc electron density map calculated with the displayed residues omitted from the model is shown as blue mesh.

FIGS. 23A-23D|EcMBP-cpGFP: effect of T203V mutation on fluorescence. (FIG. 23A) Emission spectra of 1 μM purified eGFP (filled circles), cpGFP (filled squares), MBP165-cpGFP.PPYF (open circles), and MBP165-cpGFP.PPYF+T203V (open squares) in the absence (dashed lines) or presence (solid lines) of 1 mM maltose. cpGFP is half as bright as eGFP, and the saturated MBP165-cpGFP.PPYF variants are about half as bright as cpGFP. (FIG. 23B) Titration of maltose for MBP165-cpGFP.PPYF (filled squares), and MBP165-cpGFP.PPYF+T203V (filled circles). Affinities for each protein are the same, but with different ΔF/F. (FIG. 23C) Emission spectra of 1 μM purified eGFP (filled circles), cpGFP (filled squares), MBP311-cpGFP.L2-NP (open circles), and MBP311-cpGFP.L2-NP+T203V (open squares) in the absence (dashed lines) or presence (solid lines) of 1 mM maltose. Note that mutation T203V decreases the fluorescence of both the apo-state and the saturated state of MBP311-cpGFP.L2-NP. (FIG. 23D) Titration of maltose for MBP311-cpGFP.L2-NP (filled squares), and MBP311-cpGFP.L2-NP+T203V (filled circles). Affinities for each protein are the same, but with ΔF/F slightly increased for the T203V variant.

FIG. 24A|Amino acid sequence of PfMBP171-cpGFP (SEQ ID NO:50)

FIG. 24B|Amino acid sequence of PfMBP171cpGFP.L2-FE (SEQ ID NO:51)

FIG. 25A|Amino acid sequence of PfMBP316-cpGFP (SEQ ID NO:52)

FIG. 25B|Amino acid sequence of PfMBP316-cpGFP.L1-NP (SEQ ID NO:53) FIG. 26A-26B Plot of ΔF/F for clarified lysate screen of cpGFP linker-screens at insertion points 171 (FIG. 26A) and 316 (FIG. 26B).

FIGS. 27A-27D|Plot of Beta-sheet circular dichroism (CD) signal as a function of temperature.

FIGS. 28A-28B|PfMBP Fluorescence vs. temperature. (FIG. 28A) Plot of fluorescence as a function of temperature in the presence (solid) or absence (dashed) of ligand. (FIG. 28B) Plot of ΔF/F as a function of temperature. Using the data from FIG. 27A, ΔF/F for each protein (Fbound-Fapo/Fapo) was calculated for each temperature.

FIGS. 28C-28E|Line graphs showing the function of immobilized and soluble proteins.

FIG. 29A|Amino acid sequence of EcYbeJ253-cpGFP (SEQ ID NO:62).

FIG. 29B|Amino acid sequence of EcYbeJ253-cpGFP.L1LVL2NP (SEQ ID NO:63).

FIG. 30|EcYbeJ binding curves. Plot of ΔF/F as a function of [Glutamate], μM. The first generation sensor, EcYbeJ253.L1-LV (with the A184V) mutation (grey, solid) has an affinity for glutamate of about 100 μM and a ΔF/F of 1.2. The reversion of that affinity mutation, V184A, in the L1-LV background increases affinity to 1 (grey dashed). The second generation sensor, with the L2-NP linker optimization and the A184V mutation, has a ΔF/F of at least 4 and an affinity for glutamate of about 100 μM (black solid).

FIG. 31|EcYbeJ Hema/cMyc analysis. The effect of N- and C-terminal tags on ΔF/F and glutamate affinity were determined by expressing variously tagged versions of the EcYbeJ253.L1LVL2NP protein in bacteria. The presence of the pRSET leader sequence (black) has no effect on ΔF/F (˜5) or affinity (˜120 μM), when compared to the version without a tag (grey). The addition of the cMyc tag to the C-terminus retains ΔF/F and increases affinity slightly, to 60 μM. The addition of the N-terminal hemagglutinin tag, with (green) or without (orange) the cMyc tag, decreases ΔF/F substantially.

FIGS. 32A-32B|EcYbeJ253-cpGFP.L1LVL2NP.pMinDis expressed in HEK293 cells. (FIG. 32A) Images of the sensor expressing HEK cells in the absence of glutamate (left), with 100 μM glutamate (center), and re-imaged after wash-out of glutamate with buffer (right). (FIG. 32B) By measuring the equilibrium ΔF/F with different concentrations of glutamate in the buffer, an in situ binding affinity (black) can be obtained. The surface displayed sensor has a higher affinity (3 μM) for glutamate than the soluble sensor (grey), which is about 90 μM.

FIG. 33|EcYbeJ253-cpGFP.L1LVL2NP.pMinDis expressed in neuronal culture, and responds rapidly to added glutamate (green). Red shows signal of 2.5 nM Alexa Fluor® 568 (Invitrogen, Carlsbad, CA), also in pipette.

FIG. 34A|Amino acid sequence of EcPhnD90-cpGFP (SEQ ID NO:77).

FIG. 34B|Amino acid sequence of EcPhnD90-cpGFP.L1AD+L297R+L301R (SEQ ID NO: 78).

FIGS. 35A-35C|EcPhnD90-cpGFP Binding Curves. For both the L1AD and the L1AD+L297R+L301R variants, binding was determined for (FIG. 35A) 2-aminoethylphosphonate (2AEP), (FIG. 35B) methylphosphonate (MP), and (FIG. 35C) ethylphosphonate (EP).

FIGS. 36A-36C|The crystal structures of the ligand-free (FIG. 36A), open state (with H157A mutation to the binding pocket) and the ligand-bound (FIG. 36B), closed state of EcPhnD clearly shows a large conformational change. Residues in between which cpGFP is inserted in EcPhnD90-cpGFP are marked by red spheres, in the equatorial strand (red). (FIG. 36C) Analysis of the change in Ca dihedral (ΔDihedral) clearly shows that residues for which there is the greatest ΔDihedral upon going from the open to the closed state are residues 88 (ΔDihedral=−75°), 89 (ΔDihedral=123°), and 90 (ΔDihedral=52°).

FIG. 37A|Amino acid sequence of TtGBP326-cpGFP (SEQ ID NO:91).

FIG. 37B|Amino acid sequence of TtGBP326.L1-PA (SEQ ID NO:92).

FIG. 37C|Amino acid sequence of TtGBP326.H66A (SEQ ID NO:93).

FIG. 37D|Amino acid sequence of TtGBP326.H348A (SEQ ID NO:94).

FIG. 38|TtGBP326-cpGFP Binding Curves. Plot of ΔF/F as a function of [Glucose], mM.

FIG. 39|An image showing TtGBP326-cpGFP expressed as a transgenic reporter of intracellular glucose in cultured human cells.

FIGS. 40A-40B|Are line graphs showing that the addition of extracellular glucose increases TtGBP326-cpGFP fluorescence in human cells.

FIG. 41|Amino acid sequence of Escherichia coli maltodextrin-binding protein (EcMBP) (SEQ ID NO: 105).

FIG. 42|Amino acid sequence of Pyrococcus furiosus maltose-binding protein (PfMBP) (SEQ ID NO: 106).

FIG. 43|Amino acid sequence of E. coli glutamate-binding protein (EcYbeJ) (SEQ ID NO:107).

FIG. 44|Amino acid sequence of E. coli phosphonate-binding protein (EcPhnD) (SEQ ID NO:108).

FIG. 45|Amino acid sequence of Thermus thermophilus glucose-binding protein (TtGBP) (SEQ ID NO:109).

FIG. 46|Amino acid sequence of UniProt accession number Q92N37 (SEQ ID NO: 110).

FIG. 47|Amino acid sequence of UniProt accession number D0VWX8 (SEQ ID NO:111).

FIG. 48|Amino acid sequence of UniProt accession number Q7CX36 (SEQ ID NO:112).

FIG. 49|Amino acid sequence of UniProt accession number P0AD96 (SEQ ID NO:113).

FIG. 50|Amino acid sequence of TtGBP326.L1PA.L2NP.H66A.H348A.L276V (SEQ ID NO:114).

FIG. 51|A line graph showing binding of TtGBP326.L1PA.L2NP.H66A.H348A.L276V to glucose.

FIG. 52|A line graph showing fluorescence increase upon addition of glucose to HEK293 cells expressing TtGBP326.LIPA.L2NP.H66A.H348A.L276V on their extracellular surface.

FIG. 53A-D|SF-iGluSnFR.A184S shows larger responses to visual stimuli than SF-iGluSnFR.A184V. (FIG. 53A) Two-photon standard-deviation projection of SF-iGluSnFR.A184S and A184V expressed in ferret visual cortex (A184S: 190 μm, A184V: 175 μm, scale bar 100 μm). (FIG. 53B) Trial-averaged stimulus-evoked responses (shown for ROI 1) reveal robust orientation tuning and peak amplitudes of ˜30% ΔF/F for A184S. Peak responses plotted as a function of stimulus orientation show robust selectivity with the A184S variant. For the A184V variant, stimulus-evoked fluctuations are too small (˜5% ΔF/F) to generate robust tuning plots. (FIG. 53C) Two-photon standard-deviation projection of an isolated dendritic segment with active spines revealed with SF-iGluSnFR.A184S. Individual dendritic spines are driven selectively and strongly by drifting gratings. Orientation tuning from peak responses shows large spine responses (30-50% ΔF/F) and, importantly, reveals that spines on a single dendritic branch can receive differently tuned excitatory input. (FIG. 53D) Same as in (FIG. 53C) for SF-iGluSnFR.A184V. Dendritic spine responses with A184V are weak and almost unresolvable.

FIG. 54A-D|SF-iGluSnFR.S72A permits resolution of multiple glutamate release events in cultured mouse embryonic hippocampal neurons. (FIG. 54A) Single (dashed) and averaged (solid) traces of SF-iGluSnFR.S72A (blue) and SF-iGluSnFR.A184V (red) response to 20 Hz paired electrical stimuli. (FIG. 54B) Histogram showing intensity second pulse to first pulse response. (FIG. 54C) The faster off-rate of S72A can be used to observe vesicle release depression. Higher concentrations of extracellular calcium can increase vesicle release, leading to vesicle exhaustion as the train of field pulses progresses. (FIG. 54D) The slow decay of A184V obscures this depression.

FIG. 55A-D|S72A variant shows faster bouton fluorescence signals resulting from single or trains of electrical stimulation mouse cerebellar brain slice. (FIG. 55A) Averaged response from single boutons expressing GCaMP6f (GC6f) at 2 mM [Ca²⁺]_{extracellular} (green), GC6f at 1.5 mM [Ca^{2+]}ₑ (black), SF-iGluSnFR.A184V at 1.5 mM [Ca²⁺]ₑ (A184V, red) and SF-iGluSnFR.S72A at 1.5 mM [Ca²⁺]ₑ (S72A, blue), normalized to peak response. In parenthesis the number of trials used to calculate the average. Right, summary plots of ΔF/F0, 10-90% rise time, 50% decay time and signal-to-noise-ratio (SNR). Multiple comparisons were performed with the Kruskal-Wallis test and the Dunn's multiple comparisons test. * P<0.05, ** P<0.01, **** P<0.0001. (FIG. 55B) Two-photon fluorescent image of granule cells and parallel fibers expressing A184V in cerebellum slice (GL—granule layer, ML—molecular layer). Yellow arrows indicate labeled soma of granule cells, and circle indicate boutons from parallel fibers. Bottom, example of single trial A184V fluorescence responses to 20 Hz electrical stimulation (red) and the average of 10 trials (purple). (FIG. 55C) Population average fluorescence responses to 20 Hz stimulation (n boutons=5 GC6f; n=17, A184V; n=3, S72A). Traces are normalized to the peak of the first response. (FIG. 55D) Population average of response to 100 Hz electrical stimulation (n boutons=9 GC6f; n=9, A184V; n=7, S72A) normalized to the maximum amplitude (left) or to the peak of the first response (middle), and average response of all the boutons. n is number of boutons. Black arrows indicate time of electrical stimulation.

FIG. 56A-B|High-speed two photon imaging (1016 Hz frame rate) of a neuron expressing SF-Venus-iGluSnFR. (FIG. 56A) RuBi-glutamate was uncaged for 10 msec. at each of two 5 μm spots (red arrowheads) on the dendrites. Saturation denotes the glutamate transient amplitude. Yellow line indicates locations for traces shown in (FIG. 56B). (FIG. 56B) Recorded traces at nine pixels at various distances from the uncaging focus, along the yellow line in (FIG. 56A). The traces are approximate maximum likelihood solutions recovered with the FADE algorithm. (Kazemipour et al., Proceedings of the 2017 Asilomar Conference on Signals, Systems, and Computers, October 29-November 1, Pacific Grove, CA), which incorporates dynamics having arbitrarily fast rise but slow decay. This recording is of a single uncaging event, without averaging.

FIG. 57|Circular dichroism of iGluSnFR and SF-iGluSnFR. 20 μM purified and dialyzed protein in 0.1×PBS was analyzed by circular dichroism (Chirascan, Applied Biophysics). Grey, iGluSnFR; black, SF-iGluSnFR; green, cpSFGFP; thick line, with 1 mM glutamate; thin line, no glutamate. Spectra were collected with a 1 sec. sampling time after equilibration for 2 min at each temperature. The first unfolding transition is shifted from about 50° C. to 55° C. by inclusion of the Superfolder mutations to cpGFP. Interestingly, the second transition, at about 75° C., which parallels the transition of cpSFGFP alone, is unchanged.

FIG. 58A-D|Spectra of SF-iGluSnFR. (FIG. 58A) 2-photon cross-section of purified, soluble iGluSnFR (grey) and SF-iGluSnFR (black) in the ligand-free (dashed line) and glutamate-saturated (solid line) state. Excitation (FIG. 58B), emission (FIG. 58C), and absorption spectra (FIG. 58D) of iGluSnFR (grey), SF-iGluSnFR (black), and cpSFGFP (green) with glutamate (solid line) and without (dashed line).

FIG. 59A-E|Representative images of (FIG. 59A) SF-iGluSnFR and (FIG. 59B) iGluSnFR in mouse somatosensory cortex taken at 0.9 μm/pixel, 0.126 nsec dwell time per μm, 80 mW power, prior to bleaching. 20 nl of AAV2/1.hSynapsin1.iGluSnFR or SF-iGluSnFR (identical virus titer, prepared by the same person) was injected three weeks before imaging. Contrast adjusted to 10 grayscales in both images to make original iGluSnFR observable. Mean signal-to-noise ratios (n=2 animals) are 66 vs. 14 (80 mW power) and 2.4 vs. 0.3 (5 mW power). (FIG. 59C) & (FIG. 59D) Representative images of SF-iGluSnFR and iGluSnFR taken with 5 mW power, which is more typical in live imaging conditions. (FIG. 59E) Bleaching of SF-iGluSnFR (black) and original iGluSnFR (grey) at 80 mW power and 10× zoom (0.09 μm/pixel, 1.26 nsec dwell time per μm).

FIG. 60A-B|In vitro binding affinity. (FIG. 60A) Titration of bacterially expressed iGluSnFR and SF-iGluSnFR and variants. Affinities (K_{d}) for original iGluSnFR, SF-iGluSnFR.A184S, SF-iGluSnFR.A184V, and SF-iGluSnFR. S72A are 84±7 μM, 7.5±0.4 μM, 41±7 μM, and 200±5 μM respectively. (FIG. 60B) Kinetics of glutamate binding by stopped-flow fluorescence spectroscopy. Equal volumes of 1 μM SF-iGluSnFR (A184S, A184V, or S72A) and glutamate (variable concentration) were mixed in an SX.18MV stopped-flow spectrometer (Applied Photophysics, Surrey, UK). Representative traces shown. Pseudo-first order analysis indicates that the on-rate of binding for SF-iGluSnFR.A184S, A184V, S72A are 6, 5, and 0.6 μM⁻¹ sec⁻¹, respectively. The off rates, as determined by the y-intercept, are 25 sec⁻¹, 52 sec⁻¹, and 108 sec⁻¹ respectively. Error bars are standard deviation of three measurements.

FIG. 61|Binding affinity screening. Pellets of bacterially expressed A184X variants of iGluSnFR were washed 5 times in PBS to remove bound glutamate. After freezing and thawing, pellets were clarified by centrifugation and titrated with glutamate to screen for their affinity for glutamate. There is a general trend of larger amino acids resulting in weaker affinity.

FIG. 62|Affinity of SF-iGluSnFR variants displayed on the surface of neurons. AAV2/1.hSynapsin1.SF-iGluSnFR variants (1 μl of 1E13 GC/ml) were used to infect rat hippocampal neuronal culture 3 days after culturing. After 10 days in vitro, fluorescence was monitored under continuous flow of buffer with varying concentrations of glutamate. Affinities (K_{d}) for SF-iGluSnFR.A184S, SF-iGluSnFR.A184V, and SF-iGluSnFR.S72A are 0.6, 2.1, and 34 μM respectively. Affinities for SF-Venus.A184V and SF-Azurite.A184V are 2.0 and 9 μM respectively. Bottom panel is zoom-in of top panel.

FIG. 63A-B|Rise and decay of fluorescence signal resulting from a single field stimulation (1 msec., 90 mA) in rat hippocampal culture (10 DIV, 7 DPI) in non-flowing buffer (FIG. 63B). Traces in FIG. 63A are the average of three ROIs (bottom) and three trials (top); error bars are standard deviation of those nine measurements. The large error for GCaMP6f results from back propagating action potentials, which can be seen in differences from individual ROIs.

FIG. 64|Examples of individual responses for ROIs 1, 2, and 3 (top). Responses of individual Spines #2 and #3 (from FIG. 53) (middle). Histogram showing distribution of spine responses (bottom). Response amplitudes across individual trials were consistently greater for A184S than the A184V when examining all stimulus-evoked responses.

FIG. 65A-D|Mouse neuronal culture images. The fluorescent labeling pattern and intensity of primary hippocampal neurons transduced with AAV2/1.hSynapsin1. SF-iGluSnFR.S72A or with AAV2/1.hSynapsin1-SF-iGluSnFR.A184V at DIV4 and imaged at DIV13 looked qualitatively similar for both variants and as expected for a membrane targeted protein. To resolve fast stimulus associated changes in fluorescence, a time series of 100 frames at 60 Hz during a paired-pulse stimulation paradigm was acquired. Basal fluorescence before stimulation was clearly stronger for A184V, the high affinity sensor, leading to a higher SNR (FIG. 65A and FIG. 65C). However, when dividing each frame by an average of the pre-stimulus images for both variants of SF-iGluSnFR localized spots where fluorescence increases was observed (FIG. 65B, FIG. 65D, arrows), likely representing synaptic release sites. ROIs were defined based on these spots, and fluorescence within these ROIs (background subtracted) was averaged for every image in the time series.

FIG. 66A-J|Vesicle release sites can be localized by identifying the center of stimulus-evoked SF-iGluSnFR fluorescence changes. (FIGS. 66A and 66B) Representative images of SF-iGluSnFR.S72A and SF-iGluSnFR.A184V expression in primary neuron cultures. Markers indicate the centers of Gaussians fitted to fluorescence profiles calculated across identified release sites from consecutive stimulation trials (such as shown in (FIG. 66E)). Note that the scatter of the centers of the localized release sites is substantially larger for SF-iGluSnFR.A184V (16-25 stimulation trials per experiment with inter-stimulus intervals of 20-60 s, 20 frames before and 10 frames after stimulation were recorded). (FIGS. 66C and 66D) Spots of increased fluorescence as they occur immediately after electrical stimulation when neurons are expressing SF-iGluSnFR.S72A or SF-iGluSnFR.A184V. 10 frames after the stimulus were averaged and divided by an average of 5 frames before stimulation. In this way, structures, which do not change fluorescence after simulation (background/inactive dendritic segments) will become 1. The lookup table of these images was adjusted to range from 1 to 1.5. (FIG. 66E) Line profiles calculated across the response sites shown in (FIG. 66C) and (FIG. 66D) (dashed lines) and superimposed Gaussian fits (lines). The width of the fitted Gaussian profiles were 0.57 and 1.11 μm for SF-iGluSnFR.S72A and SF-iGluSnFR.A184V, respectively. (FIG. 66F) Localization is more precise for SF-iGluSnFR. S72A. For each selected responding site (n=28-53), the mean deviation of the center of the Gaussians across the stimulation trials was calculated. These values were averaged and bar graphed for each SF-iGluSnFR variant. (FIGS. 66G and 66H) Width and amplitude of fitted Gaussian functions are significantly larger for the high affinity A184V sensor. (FIG. 66I) Gaussian fits to profiles obtained from individual (not averaged) frames after stimulus reveal the persistence of the SF-iGluSnFR.A184V variant. (FIG. 66J) Left: Gaussians fitted to the SF-iGluSnFR.A184V-mediated signal progressively broaden over time indicating that also sensor molecules remote to the site of release bind glutamate. Right: Same data as on left, but plotted as width over 4*t. The data points can be approximated by a line consistent with a diffusional spread of glutamate. The slope of the fitted line estimates the apparent (A184V-slowed) diffusion coefficient (Dapp) of synaptically released glutamate to be 4.3 μm²/s in vitro. This value is orders of magnitude smaller than the diffusion coefficient of free glutamate in solution (˜600-700 μm\hat{ }2/s) indicating that A184V not only prolongs but also substantially localizes glutamate molecules at the sites of release.

FIG. 67A-C|Annotated amino acid sequences of SF-iGluSnFR (FIG. 67A), SF-Azurite-iGluSnFR (FIG. 67C), and SF-Venus-iGluSnFR (FIG. 67B). Domains colored as indicated. Affinity modulating mutations S72A and A184V/S are indicated by orange arrow. Mutations from SF-iGluSnFR to SF-Venus-iGluSnFR and SF-Azurite-iGluSnFR indicated in red.

FIG. 68A-D|Characterization of soluble, purified SF-Azurite-iGluSnFR. (FIG. 68A) Titration of SF-Azurite-iGluSnFR yields a K_{d} of 62±11 μM, error bars are standard deviation of three measurements. Excitation (FIG. 67B), emission (FIG. 67C), and absorption (FIG. 68D) spectra of SF-Azurite-iGluSnFR (light blue) and Azurite (dark blue), with glutamate (solid line) and without (dashed line).

FIG. 69A-D|Spectra of SF-Venus-iGluSnFR. Excitation (FIG. 69A), emission (FIG. 69B), and absorbance (FIG. 69C) spectra of SF-Venus-iGluSnFR (yellow) with (solid line) and without (dashed line) glutamate. (FIG. 69D) 2-photon spectrum with SF-iGluSnFR (black) and vertical 1030 nm markup included for reference.

FIG. 70A-F Annotated amino acid sequences of the SF biosensors disclosed herein. Affinity modulating mutations S72A and A184V/S are indicated with small case letters. For SEQ ID NOs: 176-182, each domain is indicated with underlining as follows: [symbol] [symbol] ; GltI 5-253; SF-GFP 147-238; Linker; SF-GFP 1-146; GM 254-279; [symbol] [symbol] [symbol] [symbol] . SF-iGluSnFR.A184V (SEQ ID NO: 179); SF-iGluSnFR.A184S (SEQ ID NO: 177); SF-iGluSnFR.S72A (SEQ ID NO:178); SF-Venus-iGluSnFR.A184V (SEQ ID NO: 179; mutations at residues T203Y and Y65G to shift the color and at residues F46L and S72A to increase chromophore maturation are shown in lower case); SF-Venus-iGluSnFR.A184S (SEQ ID NO: 180; mutations at residues T203Y and Y65G to shift the color and at residues F46L and S72A to increase chromophore maturation are shown in lower case); SF-Venus-iGluSnFR.S72A (SEQ ID NO: 181; mutations at residues T203Y and Y65G to shift the color and at residues F46L and S72A to increase chromophore maturation are shown in lower case); SF-Azurite-iGluSnFR (SEQ ID NO: 182; mutations at residues T65S and Y66H to shift the color and at residues V150I and V224R to improve maturation and brightness are shown in lower case; Linker1 mutations: GltI-cpSFGFP connection from PILVSHNV (SEQ ID NO: 187) to PILGYHNV (SEQ ID NO: 188); Linker2 mutations: cpSFGFP-GltI connection from YNFNNPLN (SEQ ID NO: 189) to YNFNEQLN (SEQ ID NO: 190)); iDexSnFR (or SF-GlucoseSensor) (SEQ ID NO: 183); iGABASnFR (SEQ ID NO: 184; cpSFGFP was inserted after D276 of the Pf622 starting sequence. Insertion of cpSFGFP is after residue D276 of Pf622. Residues RS near the N-terminus encode BglII, and residues LQ at the C-terminus encode PstI. Mutations included in iGABASnFR include: affinity modulating hinge mutation: Pf622: F101L; Pf622-SFGFP interface: Pf622: N260A; Linker 1: SHNVY (SEQ ID NO: 191) of SFGFP to LAQVR (SEQ ID NO: 192) (SFGFP: S147L, H148A, N149Q, Y151R); Linker 2: SFGFP (SEQ ID NO: 193): F145W; Linker 2: SVLAP (SEQ ID NO: 194) of Pf622 to ANLAP (SEQ ID NO: 195) (Pf622: S277A, V278N); Binding site mutation: Pf622: F102G/Y. Underlining indicates the domain as follows: [symbol] [symbol] ; Pf622 2-276; SF-GFP 147-238; Linker; SF-GFP 1-146; Pf622 277-320; [symbol] ; [symbol] [symbol] [symbol] . Binding site mutation F102 indicated with a small case letter; and iAChSnFR (E. coli expression vector shown in SEQ ID NO:185 with the domains indicated as follows: [symbol] [symbol] [symbol] ; Thermoanaerobacter sp. X513 choline binding protein sequence; Linker regions; Circularly permuted super-folder EGFP; [symbol] [symbol] [symbol] ; and mammalian expression vector shown in SEQ ID NO: 186 with the domains indicated as follows: [symbol] [symbol] [symbol] ; Thermoanaerobacter sp. X513 choline binding protein sequence; Linker regions; Circularly permuted super-folder EGFP; [symbol] [symbol] [symbol] ).

FIG. 71|A schematic of Structure I as described herein.

### DETAILED DESCRIPTION

The present disclosure is based, at least in part, on the discovery of structures and methods related to and useful for genetically encoded biosensors. Specifically, the disclosure provides genetically encoded recombinant or chimeric peptides for use as biosensors and methods for the design, production, and use of such biosensors. As described below, these sensors can be employed (e.g., expressed) in biological systems to detect and/or monitor a wide range of target analytes (e.g., a defined, selected, and/or specific analytes) due, in part, to the signal change generated by the sensors upon binding to their respective analyte(s), which signal change allows bound and unbound sensors to be distinguished.

While the disclosure encompasses generic biosensors and methods related thereto, examples of particular binding sensors, including biosensors for detecting maltose, sucrose, maltotriose, glutamate, phosphonate, and glucose are also disclosed.

### Compositions

Provided herein are genetically encoded biosensors, i.e., nucleic acids encoding peptides, and/or the encoded peptides (e.g., isolated peptides), for use as biosensors. Biosensors herein include genetically encoded recombinant peptides containing an analyte-binding framework portion linked (e.g., operably linked) to at least one independent signaling portion, wherein the independent signaling portion is allosterically modulated or regulated by the framework portion upon interaction of the framework portion with an analyte (e.g., a defined, selected, and/or specific analyte), such that signaling from the signaling portions is altered upon interaction of the framework portion with the analyte.

In some instances, an independent signaling portion is present at a site within the framework portion that undergoes a conformational change upon interaction of the framework portion with an analyte such that the conformational change allosterically modulates or regulates signaling by the signaling portion. For example, biosensors herein can include structure I.

In some instances, signaling by the signaling portion is detectably altered upon interaction (e.g., binding) of the framework portion with an analyte. For example, signaling by the signaling portion can detectably increase or detectably decrease upon interaction (e.g., binding) of the framework portion with an analyte. In some cases, biosensors have a signal change upon binding (e.g., specific binding) to their respective analyte of at least about, for example, ±0.5, and/or an increase or decrease in signal of at least about, for example, 10%, 20%, 30%, 40%, 50%, 60%, 70%, 80%, 90%, 100%, 250%, 500%, 750%, 1000%, or more than 1000%, e.g., relative to unbound biosensor. In some increases, the level of signal change is linked to background signal. Values represented here can be converted and/or expressed into any conventional units using ordinary skill. For example, units can be expressed as ‘signal change’ (as used above), ΔF/F and/or as signal-to-noise ratio (e.g., ΔF/F multiplied by the square root of the number of photons collected). In some instances, signaling by a biosensor can be intensity based.

In some instances, biosensors herein are distinguishable from Förster resonance energy transfer, also known as fluorescence resonance energy transfer (FRET)-based sensors, which require donor and acceptor chromophores, e.g., that function in concert, in that they include independently functioning or detectable signaling portions. For example, in some instances, signaling by a first signaling portion of a biosensor herein is independent of signaling by a second signaling portion within the same or a distinct biosensor. As noted above, signaling portions are allosterically regulated by the framework portion to which they are linked upon interaction of the framework portion with an analyte (e.g., a defined, selected, and/or specific analyte).

### Framework Portions

Framework portions include genetically encoded macromolecules (e.g., proteins or peptides) that undergo conformational alteration (e.g., a structural change) upon interaction (e.g., binding) with, or to, an analyte (e.g., an analyte-binding dependent conformational alteration). For example, genetically encoded framework portions can have a first structure in the absence of an analyte (e.g., in an unbound or open state) and a second structure, that is detectably distinct (e.g., differences in structures before and after a conformational change can be observed using methods known in the art) from the first structure, in the presence of an analyte (e.g., in a bound or closed state), e.g., under physiologic conditions. In some instances, the conformational change that occurs upon interaction with an analyte (e.g., an analyte-binding dependent conformational alteration) is detectably distinct (e.g., can be observed using methods known in the art) from a conformational change that may occur for the same protein or peptide under other physiological conditions (e.g., a change in conformation induced by altered temperature, pH, voltage, ion concentration, phosphorylation).

Methods for identifying proteins or peptides that exhibit suitable conformational characteristics and/or for observing differences in structure between structures or before and after a conformational change are known in the art and/or are described herein. Such methods can include, for example, one or more of structural analysis, crystallography, NMR, EPR using Spin label techniques, Circular Dichroism (CD), Hydrogen Exchange surface Plasmon resonance, calorimetry, and/or FRET.

In some instances, framework portions can have a first structure in the absence of an analyte (e.g., in an unbound or open state) and a second structure, that is detectably distinct (e.g., can be observed using methods known in the art) from the first structure, in the presence of an analyte (e.g., in a bound or closed state), e.g., under physiologic conditions, wherein the structural change between the open and closed state can allosterically modulate an independent signaling portion recombinantly (e.g., artificially introduced) present within the framework portion (see, e.g., Structure I).

Framework portions can also interact (e.g., bind) with at least one analyte (e.g., at least one defined, specific, and/or selected analyte). In some instances, a framework portion can interact specifically with one analyte (e.g., at least one defined, specific, and/or selected analyte). In such cases, affinity of binding between the framework binding peptide and the analyte can be high or can be controlled (e.g., with millimolar, micromolar, nanomolar, or picomolar affinity). Alternatively, the single framework binding protein can bind two or more analytes (e.g., two or more defined, specific, and/or selected analytes). In such cases, affinity of binding to the two or more analytes can be the same or distinct. For example, the affinity of binding can be greater for one analyte than it is for a second or third, etc., analyte. In some instances, binding between a framework portion and an analyte (e.g., at least one defined, specific, and/or selected analyte) have an affinity of for example, 10 mM to 1 pM.

As used herein, the term “analyte” can include naturally occurring and/or synthetic sugars, amino acids, proteins (e.g., proteins, peptides, and/or antibodies), hormones, ligands, chemicals (e.g., small molecules), pharmaceuticals, nucleic acids, cells, tissues, and combinations thereof.

In some instances, biosensors can include one, two, or more framework binding portions that bind (e.g., binds specifically) a single analyte (e.g., a single defined, specific, and/or selected analyte) or distinct analytes (e.g., two or more distinct defined, specific and/or selected analytes). Alternatively or in addition, the framework portion can be chimeric. In such cases, a first part of the framework portion can be a first peptide or can be derived from a first peptide, and a second part of the framework portion can be a second peptide or can be derived from a second peptide, wherein the first a second peptides are combined to result in a single peptide.

Accordingly, framework portions can include macromolecules that undergo a conformational change upon interaction with an analyte. One non-limiting example of a suitable macromolecule is Calmodulin (CaM). CaM is in an extended shape in the absence of Ca²⁺ and in a condensed conformation in the presence of Ca²⁺ (Kuboniwa et al., Nat. Struc. Biol., 2:768-776, 1996 and Fallon and Quiocho, Structure, 11:1303-1307, 2003).

In some instances, a framework binding portion can be a bacterial protein or can be derived from a bacterial protein. Suitable bacterial proteins can include, but are not limited to, for example, periplasmic binding proteins (PBPs).

PBPs from bacteria are generally useful in the biosensors herein at least because they undergo dramatic conformational changes upon ligand binding (Ouiocho et al. Mol. Microbiol., 20:17-225, 1996). X-ray crystal structures of the apo (open) and bound (closed) forms of various PBPs reveal that these proteins have two (typically, although some have more) domains that undergo a large hinge-twist movement relative to each other in a Venus flytrap manner (Dwyer and Hellinga, Curr. Opin. Struc. Biol., 14:495-504, 2004). This conformational change has been exploited to create a number of FRET-based genetically encoded sensors (see, e.g., Deuschle et al., Pro. Sci, 14:2304-2314, 2005; Deuschle et al., Cytometry, 64:3-9, 2005; Okumoto et al., Proc. Natl. Acad. Sci. USA., 102:8740-8745, 2005; Bogner and Ludewig, J. Fluoresc., 17:350-360, 2007; and Gu et al., FEBS Letters, 580:5885-5893, 2006). In addition, the ligand-binding diversity of the PBP superfamily is large (Dwyer and Hellinga, Curr. Opin. Struc. Biol., 14:495-504, 2004).

In some instances, framework portions can include, for example, one or more of: arabinose-binding protein(s), glucose/galactose-binding protein(s), histidine-binding protein(s), maltose-binding protein(s), glutamine-binding protein(s), maltotriose-binding protein(s), RBP, ribose-binding protein(s), acetylcholine binding protein(s), choline binding protein(s), lysine binding protein(s), arginine binding protein(s), gamma aminobutyric acid (GABA) binding protein(s), ion-binding protein(s), peptide-binding protein(s), lactate-binding protein(s), histamine-binding protein(s), and/or Leucine/Isoleucine/Valine binding protein(s), including full length proteins, fragments, and/or variants thereof.

In some instances, exemplary framework portions can include: SEQ ID NO:105, which is Escherichia coli maltodextrin-binding protein (EcMBP) (UniProt accession number P0AEX9); SEQ ID NO: 106, which is Pyrococcus Furiosus maltotriose-binding protein (PfMBP) (UniProt accession number P58300); SEQ ID NO:107, which is E. coli glutamate-binding protein (EcYbeJ) (UniProt accession number Q1R3F7); SEQ ID NO:108, which is E. coli phosphonate-binding protein (EcPhnD) (UniProt accession number P37902); and/or SEQ ID NO:109, which is Thermus thermophilus glucose-binding protein (TtGBP) (UniProt accession number Q72KX2, including full length proteins, fragments, and/or variants thereof.

In some instances, exemplary framework portions can include SEQ ID NO: 110 (UniProt accession number Q92N37); SEQ ID NO:111 (UniProt accession number D0VWx8, SEQ ID NO:112 (UniProt accession number Q7CX36), and/or SEQ ID NO:113 (UniProt accession number P0AD96, including full length proteins, fragments, and/or variants thereof.

In some embodiments, exemplary framework portions can include residues 24-272 and 517-542 of SEQ ID NO: 176 (SF-iGluSnFR.A184V); residues 24-272 and 517-542 of SEQ ID NO: 177 (SF-iGluSnFR.A184S); residues 24-272 and 517-542 of SEQ ID NO: 178 (SF-iGluSnFR.S72A); residues 24-272 and 517-542 of SEQ ID NO: 179 (SF-Venus-iGluSnFR.A184V); residues 24-272 and 517-542 of SEQ ID NO: 180 (SF-Venus-iGluSnFR.A184S); residues 24-272 and 517-542 of SEQ ID NO: 181 (SF-Venus-iGluSnFR.S72A); residues 24-271 and 519-541 of SEQ ID NO: 182 (SF-Azurite-iGluSnFR); residues 24-350 and 595-664 of SEQ ID NO: 183 (iDexSnFR or SF-GlucoseSensor); residues 24-298 and 543-586 of SEQ ID NO: 184 (iGABASnFR); residues 25-99 and 348-545 of SEQ ID NO: 185 (iAChSnFR E. coli expression sequence); or residues 24-98 and 347-544 of SEQ ID NO: 186 (iAChSnFR mammalian expression sequence).

In some instances, framework portions, or biosensors, do not include signal peptides, or portions of signal peptides, that would otherwise be present in the peptide from which the framework portion is derived.

### Signaling Portions

Biosensors herein include one or more genetically encoded signaling portions (e.g., independent signaling portions) within the amino acid sequence of a framework portion at a site(s) within the framework portion that undergo(es) a conformational change upon interaction of the framework portion with an analyte (e.g., a defined, specific, and/or selected analyte).

Signaling portions (e.g., independent signaling portions) include genetically encoded molecules (e.g., peptides or proteins) that can be allosterically induced to emit a detectable signal (e.g., an analyte-binding dependent signal).

In some instances, the detectable signal is detectably distinct (e.g., can be distinguished using methods known in the art and/or disclosed herein) from a signal emitted by the molecule prior to allosteric inducement (e.g., signaling portions can emit a detectable signal in two detectably distinct states. For example, first signal can be emitted in unbound state and a second signal can be emitted in bound state). As noted above, in some instances, the detectable signal is proportional to the degree of allosteric inducement. In some instances, if two or more signaling portions are present in a biosensor, then two or more detectably distinct signals can be emitted by the biosensor.

In some instances, a genetically encoded independent signaling portion is a genetically encoded fluorescent protein (FP), e.g., a macromolecule containing a functional group (e.g., a fluorophore) that absorbs energy of a specific wavelength and re-emits energy at a different (but equally specific) wavelength, including, for example, circularly permuted FP (cpFP). In some instances, a signaling portion is a “superfolder” FP (e.g., Pedelacq et al., 2006, Nat. Biotech., 24:79-88), e.g., a circularly permuted SF FP.

As used herein, the term “fluorophore” relates to a functional group in a molecule which will absorb energy of a specific wavelength and re-emit energy at a different (but equally specific) wavelength. In some instances, fluorophore containing molecules include fluorescent proteins. The fluorophore in green fluorescent protein (GFP) includes Ser-Tyr-Gly sequence (i.e., Ser65-dehydroTyr66-Gly67), which is post-translationally modified to a 4-(p-hydroxybenzylidene)-imidazolidin-5. Exemplary genetically encoded fluorescent proteins include, but are not limited to, fluorescent proteins from coelenterate marine organisms, e.g., Aequorea victoria, Trachyphyllia geoffroyi, coral of the Discosoma genus, Renilla mulleri, Anemonia sulcata, Heteractis crispa, Entacmaea quadricolor, and/or GFP (including the variants S65T and EGFP, Renilla mulleri GFP), cyan fluorescent protein (CFP), including Cerulean, and mCerulean3 (described by Markwardt et al., PLoS ONE, 6(3) e17896.doi:10.1371/journal.pone.0017896), CGFP (CFP with Thr203Tyr: Has an excitation and emission wavelength that is intermediate between CFP and EGFP), yellow fluorescent protein (YFP, e.g., GFP-Ser65Gly/Ser72Ala/Thr203Tyr; YFP (e.g., GFP-Ser65Gly/Ser72Ala/Thr203Tyr) with Val68Leu/Gln69Lys); Citrine (i.e., YFP-Val68Leu/Gln69Met), Venus (i.e., YFP-Phe46Leu/Phe64Leu/Met153Thr/Val163Ala/Ser175Gly), PA-GFP (i.e., GFP-Val/163Ala/Thr203His), Kaede), red fluorescent protein (RFP, e.g., long wavelength fluorescent protein, e.g., DsRed (DsRed1, DsRed2, DsRed-Express, mRFP1, drFP583, dsFP593, asFP595), eqFP611, and/or other fluorescent proteins known in the art (see, e.g., Zhang et al., Nature Reviews, Molecular and Cellular Biology, 3:906-908, 2002).

As set forth above, in some instances, fluorophore containing molecules include fluorescent proteins that can be or that are circularly permutated. Circular permutation methods are known in the art (see, e.g., Baird et al., Proc. Natl. Acad. Sci., 96:11241-11246, 1999; Topell and Glockshuber, Methods in Molecular Biology, 183:31-48, 2002) as are “superfolder” (SF) proteins (e.g., Pedelacq et al., 2006, Nat. Biotech., 24:79-88) (e.g., circularly permuted SF proteins).

In some instances, single-FP sensors have a number of advantages: they preserve spectral bandwidth for multi-analyte imaging; their saturated states may be nearly as bright as the parental FP, and their ligand-free states may be arbitrarily dim, providing large theoretical fluorescence increases. This allows for much greater changes in fluorescence and thus increased signal-to-noise ratios and greater resistance to photobleaching artifacts (Tian et al., Nat. Methods, 6:875-881, 2009).

In some instances, issues arising from long-term effects such as gene regulation and protein expression and degradation can be identified by simply fusing the intensity-based sensor to a another fluorescent protein of different color, to serve as a reference channel.

In some instances, biosensors can include circularly permuted YFP (cpYFP) as a cpFP. cpYFP has been used as a reporter element in the creation of sensors for H₂O₂ (HyPer) (Belousov et al., Nat. Methods, 3:281-286, 2006), cGMP (FlincG) (Nausch et al., Proc. Natl. Acad. Sci. USA., 105:365-370, 2008), ATP:ADP ratio (Perceval) (Berg et al., Nat. Methods., 105:365-370, 2008), and calcium ions (Nakai et al., Nat. Biotechno., 19:137-141, 2001), including full length, fragments, and/or variants thereof.

In some embodiments, exemplary sensor portions can include residues 273-516 of SEQ ID NO: 176 (SF-iGluSnFR.A184V); residues 273-516 of SEQ ID NO: 177 (SF-iGluSnFR.A184S); residues 273-516 of SEQ ID NO: 178 (SF-iGluSnFR.S72A); residues 273-516 of SEQ ID NO: 179 (SF-Venus-iGluSnFR.A184V); residues 273-516 of SEQ ID NO: 180 (SF-Venus-iGluSnFR.A184S); residues 273-516 of SEQ ID NO: 181 (SF-Venus-iGluSnFR.S72A); residues 272-518 of SEQ ID NO: 182 (SF-Azurite-iGluSnFR); residues 351-594 of SEQ ID NO: 183 (iDexSnFR or SF-GlucoseSensor); residues 299-544 of SEQ ID NO: 184 (iGABASnFR); residues 104-343 of SEQ ID NO: 185 (iAChSnFR E. coli expression sequence); or residues 103-342 of SEQ ID NO: 186 (iAChSnFR mammalian expression sequence).

### Linker Portions

As shown in Structure I, biosensors herein can optionally include one or more genetically encoded linkers positioned between or operably linking the framework portion and the signaling portion. Linker portions can include at least one naturally occurring or synthetic amino acid (discussed below) as exemplified by SEQ ID NOs: 9-49, 54-61, 64-76, 79-90, 95-104. In some instances, linker can include one or more of SEQ ID NOs: 9-49, 54-61, 64-76, 79-90, 95-104, and/or portions of SEQ ID NOs: 9-49, 54-61, 64-76, 79-90, 95-104. For example, linkers can include, but are not limited to, one or more of: PxSHNVY (SEQ ID NO:114), xPSHNVY (SEQ ID NO:115), xxSHNVY (SEQ ID NO:116), xxSHNVF (SEQ ID NO:117), PxSHNVF (SEQ ID NO:118), PxSYNVF (SEQ ID NO:119), xxSYNVF (SEQ ID NO:120), PxSYNVF (SEQ ID NO:121), xxSYNVF (SEQ ID NO:122), PxSxNVY (SEQ ID NO:123), PxSHxVY (SEQ ID NO:124), PxSHNxY (SEQ ID NO:125), PxSHNVx (SEQ ID NO:126), FNxxY (SEQ ID NO:127), FNxY (SEQ ID NO:128), FNY (SEQ ID NO:129), FxY (SEQ ID NO:130), xxY (SEQ ID NO:131), WxY (SEQ ID NO:132), xKY, (SEQ ID NO:133), FNPxY (SEQ ID NO:134), FNxPY (SEQ ID NO:135), HNS (SEQ ID NO:136), GGS (SEQ ID NO:137), xxS (SEQ ID NO:138), xxK (SEQ ID NO:139), GGK (SEQ ID NO:140), PXS (SEQ ID NO:141), xPS (SEQ ID NO:142), Px (SEQ ID NO:143), xP (SEQ ID NO:144), IxxS (SEQ ID NO:145), NxPK (SEQ ID NO:146), NPcK (SEQ ID NO:147), PPxSH (SEQ ID NO:148), PPxxSH (SEQ ID NO:149), PPPxSH (SEQ ID NO:150), PPxPSH (SEQ ID NO:151), xxSH (SEQ ID NO:152), PPxx (SEQ ID NO:153), FNxKN (SEQ ID NO:154), FNxxKN (SEQ ID NO:155), FNxPKN (SEQ ID NO:156), FNPxKN (SEQ ID NO:157), FNxx (SEQ ID NO:158), N, ADGSSH (SEQ ID NO:159), ADxxSH (SEQ ID NO:160), ADxPSH (SEQ ID NO:161), ADPxSH (SEQ ID NO:162), ADxx (SEQ ID NO:163), ADxxSH (SEQ ID NO:164), FNPG (SEQ ID NO:165), FNxxPG (SEQ ID NO:166), xxPG (SEQ ID NO:167), FNxx (SEQ ID NO:168), FNPx (SEQ ID NO:169), KYxxSH (SEQ ID NO:170), KYPxSH (SEQ ID NO:171), KYxPSH (SEQ ID NO:172), FxxP (SEQ ID NO:173), FNxP (SEQ ID NO:174), and/or FNPx (SEQ ID NO:175), where “x” indicates any amino acid.

In some embodiments, exemplary linker portions can include residues 365-370 of SEQ ID NO: 176 (SF-iGluSnFR.A184V); residues 365-370 of SEQ ID NO: 177 (SF-iGluSnFR.A184S); residues 365-370 of SEQ ID NO: 178 (SF-iGluSnFR.S72A); residues 365-370 of SEQ ID NO: 179 (SF-Venus-iGluSnFR.A184V); residues 365-370 of SEQ ID NO: 180 (SF-Venus-iGluSnFR.A184S); residues 365-370 of SEQ ID NO: 181 (SF-Venus-iGluSnFR.S72A); residues 365-370 of SEQ ID NO: 182 (SF-Azurite-iGluSnFR); residues 443-448 of SEQ ID NO: 183 (iDexSnFR or SF-GlucoseSensor); residues 391-396 of SEQ ID NO: 184 (iGABASnFR); residues 100-103 and 344-347 of SEQ ID NO: 185 (iAChSnFR E. coli expression sequence); or residues 99-102 and 343-346 of SEQ ID NO: 186 (iAChSnFR mammalian expression sequence).

### Exemplary Biosensor Constructs

As noted above, biosensors herein include genetically encoded biosensors, i.e., nucleic acids encoding biosensors, and/or the encoded biosensors (e.g., isolated biosensors), for use as biosensors. In some instances, nucleic acids encoding biosensors include isolated nucleic acids. In some instances, the portion of a nucleic acid encoding a biosensor can include a single reading frame encoding the biosensor. For example, a biosensor can be encoded by a portion of a nucleic acid that falls within a start codon and a stop codon. In some instances, biosensors are isolated (e.g., biosensors are substantially free of contaminating and/or non-biosensor components).

In some instances, biosensors can include, for example, one or more framework portions selected from the group consisting of: arabinose-binding protein(s), glucose/galactose-binding protein(s), histidine-binding protein(s), maltose-binding protein(s), maltotriose-binding protein(s), glutamine-binding protein(s), RBP, ribose-binding protein(s), acetylcholine binding protein(s), choline binding protein(s), lysine binding protein(s), arginine binding protein(s), gamma aminobutyric acid (GABA) binding protein(s), ion-binding protein(s), peptide-binding protein(s), lactate-binding protein(s), histamine-binding protein(s), and/or Leucine/Isoleucine/Valine binding protein(s), including full length proteins, fragments, and/or variants thereof, including full length proteins, fragments and/or variants thereof, and at least one independent signaling portion present at a site within the framework portion that undergoes a conformational change upon interaction of the framework portion with an analyte.

In some instances, biosensors can include, for example, one or more framework portions selected from the group consisting of: SEQ ID NO:105, which is Escherichia coli maltodextrin-binding protein (EcMBP) (UniProt accession number P0AEX9); SEQ ID NO: 106, which is Pyrococcus Furiosus maltose-binding protein (PfMBP) (UniProt accession number P58300); SEQ ID NO:107, which is E. coli glutamate-binding protein (EcYbeJ) (UniProt accession number Q1R3F7); SEQ ID NO:108, which is E. coli phosphonate-binding protein (EcPhnD) (UniProt accession number P37902); and/or SEQ ID NO:109, which is Thermus thermophilus glucose-binding protein (TtGBP) (UniProt accession number Q72KX2), including full length proteins, fragments and/or variants thereof, and at least one independent signaling portion present at a site within the framework portion that undergoes a conformational change upon interaction of the framework portion with an analyte.

In some instances, biosensors can include, for example, one or more framework portions selected from the group consisting of: SEQ ID NO: 110 (UniProt accession number Q92N37); SEQ ID NO:111 (UniProt accession number D0VWx8, SEQ ID NO:112 (UniProt accession number Q7CX36), and/or SEQ ID NO:113 (UniProt accession number P0AD96), including full length proteins, fragments and/or variants thereof, and at least one independent signaling portion present at a site within the framework portion that undergoes a conformational change upon interaction of the framework portion with an analyte.

In some instances, biosensors include any one or more:
Maltose biosensors SEQ ID NOs: 1-8 (e.g., Escherichia coli maltodextrin-binding protein (EcMBP)) or SEQ ID NOs: 50-53 (e.g., Pyrococcus furiosus maltose-binding protein (PfMBP)), including full length proteins, fragments and/or variants thereof;
Glutamate biosensors SEQ ID NOs: 62-63 (e.g., E. coli glutamate-binding protein (EcYbeJ)) or SEQ ID NOs: 176-182, including full length proteins, fragments and/or variants thereof;
Phosphonate biosensors SEQ ID NOs: 77-78 (e.g., E. coli phosphonate-binding protein (EcPhnD)), including full length proteins, fragments and/or variants thereof;
Glucose biosensors SEQ ID NOs: 91-94 (e.g., Thermus thermophilus glucose-binding protein (TtGBP)) and SEQ ID NO: 183, including full length proteins, fragments and/or variants thereof;
GABA biosensors SEQ ID NO: 184, including full length proteins, fragments and/or variants thereof; and/or
ACh biosensors SEQ ID NOs: 185 & 186, including full length proteins, fragments and/or variants thereof.

In some instances, nucleic acids encoding, and/or amino acid sequences of, any of the framework portions, signaling portions, linker portions, or the entire biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) disclosed herein can be modified to generate fragments (e.g., truncated peptides) and/or variants (e.g., peptides with a defined sequence homology to the peptides disclosed herein). Variants can include framework portions, signaling portions, linker portions, or biosensors with amino acid sequences with homology to the framework portions, signaling portions, linker portions, or biosensors disclosed herein and/or truncated forms of the framework portions, signaling portions, linker portions, or biosensors herein. In some instances, truncated forms of the framework portions, signaling portions, linker portions, or biosensors herein can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49, 50, 50-100, 101-150, fewer amino acids than the framework portions, signaling portions, linker portions, and/or biosensors herein, e.g., wherein the truncated biosensor variants retain at least at portion of the binding and/or signaling properties of same biosensor without truncation (e.g., at least 50%, 60%, 70%, 80%, 90%, or 100% of the binding and/or signaling properties of the same biosensor without truncation). In addition, truncations can be made at the amino-terminus, the carboxy-terminus, and/or within the body of the framework portions, signaling portions, linker portions, and/or biosensors herein.

While variants are generally observed and discussed at the amino acid level, the actual modifications are typically introduced or performed at the nucleic acid level. For example, variants with 95%, 96%, 97%, 98, or 99% sequence identity to SEQ ID NOs: 1, 2, 3, 4, 5, 6, 7, 8, 50, 51, 52, 53, 62, 63, 77, 78, 91, 92, 93, 94, and/or 176-186 can be generated by modifying the nucleic acids encoding SEQ ID NOs: 1, 2, 3, 4, 5, 6, 7, 8, 50, 51, 52, 53, 62, 63, 77, 78, 91, 92, 93, 94, and/or 176-186 using techniques (e.g., cloning techniques) known in the art and/or that are disclosed herein.

As with all peptides, polypeptides, and proteins, including fragments thereof, it is understood that modifications to the amino acid sequence can occur that do not alter the nature or function of the peptides, polypeptides, or proteins. Such modifications include conservative amino acids substitutions and are discussed in greater detail below.

The peptides, polypeptides, and proteins, including fragments thereof, provided herein are biosensors whose activity can be tested or verified, for example, using the in vitro and/or in vivo assays described herein.

In some instances, any of the framework portions, signaling portions, or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) described herein can be modified and varied so long as their desired function is maintained. For example, the polypeptides can be modified as long as the resulting variant polypeptides have the same or better characteristics as the polypeptide from which they derived. For example, the variants can have the same or better affinity for their respective analyte.

In some instances, the interacting face of a modified peptide can be the same (e.g., substantially the same) as an unmodified peptide (methods for identifying the interacting face of a peptide are known in the art (Gong et al., BMC: Bioinformatics, 6:1471-2105 (2007); Andrade and Wei et al., Pure and Appl. Chem., 64(11):1777-1781 (1992); Choi et al., Proteins: Structure, Function, and Bioinformatics, 77(1):14-25 (2009); Park et al., BMC: and Bioinformatics, 10:1471-2105 (2009)), e.g., to maintain binding to an analyte. Alternatively, amino acids within the interacting face can be modified, e.g., to decrease binding to an analyte and/or to change analyte specificity.

The interacting face of a peptide is the region of the peptide that interacts or associates with other molecules (e.g., other proteins). Generally, amino acids within the interacting face are naturally more highly conserved than those amino acids located outside the interacting face or interface regions of a protein. In some instances, an amino acid within the interacting face region of any of the framework portions or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) disclosed herein can be the same as the amino acid shown in any of the framework portions or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) disclosed herein or can be include conservative amino acid substitutions. In some instances, an amino acid within the interacting face region any of the framework portions or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) disclosed herein can be substituted with an amino acid that increases the interaction between the framework portion or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) and an analyte.

In some instances, genetically encoded biosensors can include peptides that have at least 80, 85, 90, 95, 96, 97, 98, 99 percent identity to the framework portions, signaling portions, or the biosensor sequence (e.g., SEQ ID NOs: 1, 2, 3, 4, 5, 6, 7, 8, 50, 51, 52, 53, 62, 63, 77, 78, 91, 92, 93, 94, and/or 176-186) (e.g., any amino acid sequence) described herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math, 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-10 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Amino acid sequence modifications typically fall into one or more of three classes: substitutional, insertional, or deletional modifications. Insertions include amino and/or terminal fusions as well as intra-sequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions can be made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place. In some instances, substitutions can be conservative amino acid substitutions. In some instances, variants herein can include one or more conservative amino acid substitutions. For example, variants can include 1, 2, 3, 4, 5, 6, 7, 8, 9, 10, 11, 12, 13, 14, 15, 16, 17, 18, 19, 20, 20-30, 30-40, or 40-50 conservative amino acid substitutions. Alternatively, variants can include 50 or fewer, 40 or fewer, 30 or fewer, 20 or fewer, 10 or fewer, 9 or fewer, 8 or fewer, 7 or fewer, 6 or fewer, 5 or fewer, 4 or fewer, 3 or fewer, or 2 or fewer conservative amino acid substitutions. Such substitutions generally are made in accordance with the following Table 1 and are referred to as conservative substitutions. Methods for predicting tolerance to protein modification are known in the art (see, e.g., Guo et al., Proc. Natl. Acad. Sci., USA, 101(25):9205-9210 (2004)).

TABLE 1
Conservative Amino Acid Substitutions

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

In some instances, substitutions are not conservative. For example, an amino acid can be replaced with an amino acid that can alter some property or aspect of the peptide. In some instances, non-conservative amino acid substitutions can be made, e.g., to change the structure of a peptide, to change the binding properties of a peptide (e.g., to increase or decrease the affinity of binding of the peptide to an analyte and/or to alter increase or decrease the binding specificity of the peptide).

Modifications, including the specific amino acid substitutions, are made by known methods. By way of example, modifications are made by site-specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

### Nucleic Acids

The disclosure also features nucleic acids encoding the biosensors (e.g., SEQ ID NOs: 1, 2, 3, 4, 5, 6, 7, 8, 50, 51, 52, 53, 62, 63, 77, 78, 91, 92, 93, 94, and/or 176-186) described herein, including variants and/or fragments of the biosensors (e.g., variants and/or fragments of SEQ ID NOs: 1, 2, 3, 4, 5, 6, 7, 8, 50, 51, 52, 53, 62, 63, 77, 78, 91, 92, 93, 94, and/or 176-186). These sequences include all degenerate sequences related to the specific polypeptide sequence, i.e., all nucleic acids having a sequence that encodes one particular polypeptide sequence as well as all nucleic acids, including degenerate nucleic acids, encoding the disclosed variants and derivatives of the polypeptide sequences. Thus, while each particular nucleic acid sequence may not be written out herein, it is understood that each and every sequence is in fact disclosed and described herein through the disclosed polypeptide sequences.

In some instances, nucleic acids can encode biosensors with 95, 96, 97, 98, or 99 identity to SEQ ID NOs: 1, 2, 3, 4, 5, 6, 7, 8, 50, 51, 52, 53, 62, 63, 77, 78, 91, 92, 93, 94, and/or 176-186.

In some instances, nucleic acids can encode SEQ ID NOs: 1, 2, 3, 4, 5, 6, 7, 8, 50, 51, 52, 53, 62, 63, 77, 78, 91, 92, 93, 94, and/or 176-186 containing 1, 2, 3, 4, 5, 6, 7, 8, 9, 10, 11, 12, 13, 14, 15, 16, 17, 18, 19, 20, 20-30, 30-40, or 40-50 conservative amino acid substitutions.

In some instances, nucleic acids can encode SEQ ID NOs: 1, 2, 3, 4, 5, 6, 7, 8, 50, 51, 52, 53, 62, 63, 77, 78, 91, 92, 93, 94, and/or 176-186 containing 50 or fewer, 40 or fewer, 30 or fewer, 20 or fewer, 10 or fewer, 9 or fewer, 8 or fewer, 7 or fewer, 6 or fewer, 5 or fewer, 4 or fewer, 3 or fewer, or 2 or fewer conservative amino acid substitutions

Also provided herein are vectors comprising the biosensors (e.g, SEQ ID NOs: 1, 2, 3, 4, 5, 6, 7, 8, 50, 51, 52, 53, 62, 63, 77, 78, 91, 92, 93, 94, and/or 176-186) described herein, including variants and/or fragments of the biosensors (e.g, SEQ ID NOs: 1, 2, 3, 4, 5, 6, 7, 8, 50, 51, 52, 53, 62, 63, 77, 78, 91, 92, 93, 94, and/or 176-186). For example:

Vectors can include nucleic acids that encode biosensors with 95, 96, 97, 98, or 99 identity to SEQ ID NOs: 1, 2, 3, 4, 5, 6, 7, 8, 50, 51, 52, 53, 62, 63, 77, 78, 91, 92, 93, 94, and/or 176-186.

Vectors can include nucleic acids that encode SEQ ID NOs: 1, 2, 3, 4, 5, 6, 7, 8, 50, 51, 52, 53, 62, 63, 77, 78, 91, 92, 93, 94, and/or 176-186 containing 1, 2, 3, 4, 5, 6, 7, 8, 9, 10, 11, 12, 13, 14, 15, 16, 17, 18, 19, 20, 20-30, 30-40, or 40-50 conservative amino acid substitutions.

Vectors can include nucleic acids that encode SEQ ID NOs: 1, 2, 3, 4, 5, 6, 7, 8, 50, 51, 52, 53, 62, 63, 77, 78, 91, 92, 93, 94, and/or 176-186 containing 50 or fewer, 40 or fewer, or fewer, 20 or fewer, 10 or fewer, 9 or fewer, 8 or fewer, 7 or fewer, 6 or fewer, 5 or fewer, 4 or fewer, 3 or fewer, or 2 or fewer conservative amino acid substitutions

Examples of suitable vectors include, but are not limited to, plasmids, artificial chromosomes, such as BACs, YACs, or PACs, and viral vectors. As used herein, vectors are agents that transport the disclosed nucleic acids into a cell without degradation and, optionally, include a promoter yielding expression of the nucleic acid molecule in the cells into which it is delivered.

Viral vectors can include, for example, Adenovirus, Adeno-associated virus, herpes virus, Vaccinia virus, Polio virus, Sindbis, and other RNA viruses, including these viruses with the HIV backbone. Any viral families which share the properties of these viruses which make them suitable for use as vectors are suitable. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating Vaccinia virus vectors.

Non-viral based vectors can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clontech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), enhancers derived from a eukaryotic cell viruses can be used. Examples of such can include the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Promoters of this type can include the CMV promoter, the SV40 promoter, the β-actin promoter, the EF1α promoter, and the retroviral long terminal repeat (LTR).

The provided vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

The disclosure further provides cells comprising the biosensors (e.g., SEQ ID NOs: 1, 2, 3, 4, 5, 6, 7, 8, 50, 51, 52, 53, 62, 63, 77, 78, 91, 92, 93, 94, and/or 176-186) described herein, including variants and/or fragments of the biosensors (e.g., SEQ ID NOs: 1, 2, 3, 4, 5, 6, 7, 8, 50, 51, 52, 53, 62, 63, 77, 78, 91, 92, 93, 94, and/or 176-186). Cells can include, for example, eukaryotic and/or prokaryotic cells. For example, cells can include, but are not limited to cells of E. coli, Pseudomonas, Bacillus, Streptomyces; fungi cells such as yeasts (Saccharomyces, and methylotrophic yeast such as Pichia, Candida, Hansenula, and Torulopsis); and animal cells, such as CHO, R1.1, B-W and LM cells, African Green Monkey kidney cells (for example, COS 1, COS 7, BSC1, BSC40, and BMT10), insect cells (for example, Sf9), human cells and plant cells. Suitable human cells can include, for example, HeLa cells or human embryonic kidney (HEK) cells. In general, cells that can be used herein are commercially available from, for example, the American Type Culture Collection (ATCC), P.O. Box 1549, Manassas, VA 20108. See also F. Ausubel et al., Current Protocols in Molecular Biology, John Wiley & Sons, New York, NY, (1998).

Optionally, the biosensors (e.g., SEQ ID NOs: 1, 2, 3, 4, 5, 6, 7, 8, 50, 51, 52, 53, 62, 63, 77, 78, 91, 92, 93, 94, and/or 176-186) described herein, including variants and/or fragments of the biosensors (e.g., SEQ ID NOs: 1, 2, 3, 4, 5, 6, 7, 8, 50, 51, 52, 53, 62, 63, 77, 78, 91, 92, 93, 94, and/or 176-186) can be located in the genome of the cell (e.g., can be stably expressed in the cell) or can be transiently expressed in the cell.

Methods of making the provided cells are known and the method of transformation and choice of expression vector will depend on the host system selected. Transformation and transfection methods are described, e.g., in F. Ausubel et al., Current Protocols in Molecular Biology, John Wiley & Sons, New York, NY, (1998), and, as described above, expression vectors may be chosen from examples known in the art.

There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

By way of example, the provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004). The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003). The provided polypeptides can be delivered by tegument aggregates. Methods for making and using tegument aggregates are described in International Publication No. WO 2006/110728.

Also provided are transgenic animals comprising one or more cells the biosensors (e.g, SEQ ID NOs: 1, 2, 3, 4, 5, 6, 7, 8, 50, 51, 52, 53, 62, 63, 77, 78, 91, 92, 93, 94, and/or 176-186) described herein, including variants and/or fragments of the biosensors (e.g, SEQ ID NOs: 1, 2, 3, 4, 5, 6, 7, 8, 50, 51, 52, 53, 62, 63, 77, 78, 91, 92, 93, 94, and/or 176-186). As used herein, the term animal refers to non-human animals, including, mammals, amphibians and birds. Specifically, examples include sheep, feline, bovines, ovines, pigs, horses, rabbits, guinea pigs, mice, hamsters, rats, non-human primates, and the like. As used herein, transgenic animal refers to any animal, in which one or more of the cells of the animal contain a heterologous nucleic acid. The heterologous nucleic acid can be introduced using known transgenic techniques. The nucleic acid is introduced into the cell, directly or indirectly. For example, the nucleic acid can be introduced into a precursor of the cell or by way of deliberate genetic manipulation, such as by microinjection or by infection with a recombinant virus. The nucleic acid may be integrated within a chromosome, or it may be an extrachromosomally replicating DNA.

Methods for making transgenic animals using a variety of transgenes have been described in Wagner et al. (1981) Proc. Nat. Acad. Sci. USA, 78:5016-5020; Stewart et al. (1982) Science, 217:1046-1048; Constantini et al. (1981) Nature, 294:92-94; Lacy et al. (1983) Cell, 34:343-358; McKnight et al. (1983) Cell, 34:335-341; Brinstar et al. (1983) Nature, 306:332-336; Palmiter et al. (1982) Nature, 300:611-615; Palmiter et al. (1982) Cell, 29:701-710; and Palmiter et al. (1983) Science, 222:809-814. Such methods are also described in U.S. Pat. Nos. 6,175,057; 6,180,849; and 6,133,502.

By way of example, the transgenic animal can be created by introducing a nucleic acid into, for example, an embryonic stem cell, an unfertilized egg, a fertilized egg, a spermatozoon or a germinal cell containing a primordial germinal cell thereof, preferably in the embryogenic stage in the development of a non-human mammal (more preferably in the single-cell or fertilized cell stage and generally before the 8-cell phase). The nucleic acid can be introduced by known means, including, for example, the calcium phosphate method, the electric pulse method, the lipofection method, the agglutination method, the microinjection method, the particle gun method, the DEAE-dextran method and other such method. Optionally, the nucleic acid is introduced into a somatic cell, a living organ, a tissue cell or other cell by gene transformation methods. Cells including the nucleic acid may be fused with the above-described germinal cell by a commonly known cell fusion method to create a transgenic animal.

For embryonic stem (ES) cells, an ES cell line may be employed, or embryonic cells may be obtained freshly from a host, e.g., mouse, rat, guinea pig, and the like. Such cells are grown on an appropriate fibroblast-feeder layer or grown in the presence of appropriate growth factors, such as leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the nucleic acid. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. The chimeric animals are screened for the presence of the nucleic acid, and males and females having the modification are mated to produce homozygous progeny transgenic animals.

Kits comprising one or more containers and the nucleic acid sequences, polypeptides, vectors, cells, provided herein, or combinations thereof, are also provided. For example, provided is a kit comprising (i) a nucleic acid sequence encoding a biosensor described herein (e.g, one or more of SEQ ID NOs: 1, 2, 3, 4, 5, 6, 7, 8, 50, 51, 52, 53, 62, 63, 77, 78, 91, 92, 93, 94, and/or 176-186), including variants and/or fragments of the biosensor (e.g, variants or fragments of one or more of SEQ ID NOs: 1, 2, 3, 4, 5, 6, 7, 8, 50, 51, 52, 53, 62, 63, 77, 78, 91, 92, 93, 94, and/or 176-186), (ii) a polypeptide comprising a biosensor described herein (e.g, one or more of SEQ ID NOs: 1, 2, 3, 4, 5, 6, 7, 8, 50, 51, 52, 53, 62, 63, 77, 78, 91, 92, 93, 94, and/or 176-186), including variants and/or fragments of the biosensor (e.g, variants or fragments of one or more of SEQ ID NOs: 1, 2, 3, 4, 5, 6, 7, 8, 50, 51, 52, 53, 62, 63, 77, 78, 91, 92, 93, 94, and/or 176-186), (iii) a vector comprising the nucleic acid of (i), (iv) a cell comprising the nucleic acid or (i) and/or the polypeptide of (ii), (v) a cell comprising the vector of (iii). The kit can comprise any combination of (i)-(v). Optionally, the kit further comprises reagents for using the nucleic acid or peptide biosensors, vectors, and/or cells. For example, if the kit comprises cells, the kit may also comprise cell culture medium. Optionally, the kit further comprises instructions for use. Optionally, the kit further comprises a GPCR, a GPCR-encoding nucleic acid sequence.

### Design and Production/Manufacture Methods

Using the methods described herein, it is possible to design, produce, and/or adapt genetically encoded biosensors to assays for a variety of classes of analytes. The provided materials and methods facilitate the discovery of new compounds targeting a wide array of protein targets, including but not limited to: endogenous targets responsible for disease state progression, targets on pathogens for treating infectious diseases, and endogenous targets to be avoided (thus screening early for potential drug side effects and toxicity).

Methods herein provide systematic and generic approaches for the design and production of genetically encoded recombinant peptides containing an analyte-binding framework portion linked (e.g., operably linked) to a signaling portion, wherein the signaling portion is allosterically modulated or regulated by the framework portion upon interaction of the framework portion with an analyte. Generally, methods include: (i) selecting one or more target analytes; (ii) selecting a framework portion (e.g., a PBP) that interacts with (e.g., interacts specifically with) or binds to (e.g., binds specifically to) the target analyte and that undergoes a conformational change upon interacting with or binding to the analyte; (iii) identifying sites or amino acid positions within the framework portion (e.g., the PBP) where the conformational change occurs; and (iv) inserting or cloning a signaling portion into the site or amino acid position identified in (iii). Methods can, optionally, further include: (v) modifying or optimizing linker sequences between the framework portion and the signaling portion, for example, by genetic manipulation (e.g., by point mutation); (vi) modifying or optimizing analyte binding; (vii) modifying the signal generated by the biosensor; and/or (viii) cloning the biosensor into a suitable vector.

In some instances: (iii) includes identification of insertion sites by analysis of the structure (e.g., crystal structure) of the selected framework portion (e.g., the selected PBP) in one or both of its open and closed states to determine amino acid positions at which analyte-binding dependent structural changes occur. In instances where structures for both open and closed states are not available, analysis can be conducted by analogy to a structurally similar framework portion (e.g., PBP); (iv) includes cloning a signaling portion (e.g., a cpFP) at the site identified in (iii) such that the analyte-binding dependent structural change observed in (iii) will result in a conformational change in the signaling portion (e.g., the cpFP) and allosteric modulation of the signaling portion; (v) includes generating a library of mutants of biosensors with distinct linker sequences (e.g., by point mutation), screening the library of mutants to identify mutants with enhanced properties (e.g., improved signal-to-noise ratio), and selecting mutants with enhanced properties (e.g., improved signal-to-noise ratio); (vi) includes increasing or decreasing binding or affinity of the framework portion to the analyte, e.g., by modifying amino acids in the interacting face of the framework portion or regions within the framework portion that are critical for analyte binding; (vii) includes increasing or decreasing signal emission by the signaling portion and/or changing the color of the signal where the signaling portion is a FP (e.g., a cpFP). Methods including (i)-(viii) are exemplified in the Examples section herein.

### Methods of Use

The disclosure further provides methods for using the biosensors disclosed herein (e.g., one or more of SEQ ID NOs: 1, 2, 3, 4, 5, 6, 7, 8, 50, 51, 52, 53, 62, 63, 77, 78, 91, 92, 93, 94, and/or 176-186), including variants and/or fragments of the biosensor (e.g., variants or fragments of one or more of SEQ ID NOs: 1, 2, 3, 4, 5, 6, 7, 8, 50, 51, 52, 53, 62, 63, 77, 78, 91, 92, 93, 94, and/or 176-186)) to detect analytes, e.g., in biological systems. Such methods can include, for example:
Use of a maltose biosensor disclosed herein (e.g., one or more of SEQ ID NOs: 1, 2, 3, 4, 5, 6, 7, 8, 50, 51, 52, and/or 53 including variants and/or fragments of SEQ ID NOs: 1, 2, 3, 4, 5, 6, 7, 8, 50, 51, 52, and/or 53) to detect maltose, e.g., in a biological system;
Use of a glutamate biosensor disclosed herein (e.g., one or more of SEQ ID NOs: 62, 63, and/or 176-182 including variants and/or fragments of SEQ ID NOs: 62, 63, and/or 176-182) to detect glutamate, e.g., in a biological system;
Use of a phosphonate biosensor disclosed herein (e.g., one or more of SEQ ID NOs: 77 and/or 78 including variants and/or fragments of SEQ ID NOs: 77 and/or 78) to detect phosphonate, e.g., in a biological system; and/or
Use of a glucose biosensor disclosed herein (e.g., one or more of SEQ ID NOs: 91, 92, 93, 94, and/or 183 including variants and/or fragments of SEQ ID NOs: 91, 92, 93, 94, and/or 183) to detect glucose, e.g., in a biological system.
Use of a GABA biosensor disclosed herein (e.g., SEQ ID NO: 184 including variants and/or fragments of SEQ ID NO: 184) to detect GABA, e.g., in a biological system.
Use of an ACh biosensor disclosed herein (e.g., one or more of SEQ ID NOs: 185 and/or 186 including variants and/or fragments of SEQ ID NOs: 185 and/or 186) to detect ACh, e.g., in a biological system.

Techniques for performing such methods are known in the art and/or are exemplified herein. For example, methods can include introducing one or more biosensors into a biological system (e.g., a cell); expressing the one or more biosensors in the biological system (e.g., the cell); monitoring the signal emitted by the expressed biosensor in the biological system; and correlating the signal emitted by the expressed biosensor in the biological system with a level of the analyte in the biological system.

### EXAMPLES

The invention is further described in the following examples, which do not limit the scope of the invention described in the claims.

### Example 1: Maltose Indicators

Genetically encoded maltose indicators were generated using Escherichia coli maltodextrin-binding protein (EcMBP) as a framework and either circularly permuted (3-lactamase (cpB1a) or circularly permuted fluorescent protein (cpFP) as a signal. Data describe below suggest that cpB1a and cpFP are not interchangeable.

Allosteric coupling of ligand binding to fluorescence was hypothesized to require:
i) that the site in into which cpGFP is inserted have the capacity to transduce the global conformational change the scaffold protein (EcMBP in this example) to the local environment of the chromophore in cpGFP; and
ii) that the local environment of the chromophore (e.g., linkers) be optimized to maximize the difference in emission between unbound (apo) and the bound (in this example maltose-bound) states.

### Example 1A: Identification of cpGFP Insertion Sites in EcMBP

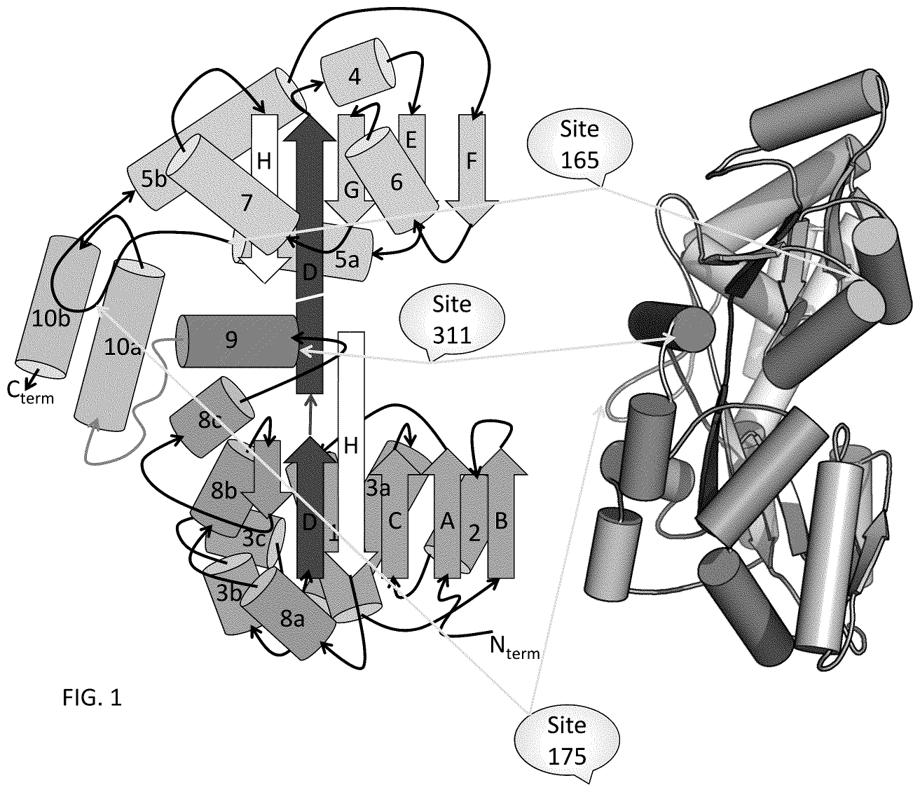

Potential insertion sites were identified using the crystal structures of the maltose-bound, closed form of EcMBP (Ouiocho et al., Structure, 5:997-1015, 1997) and the ligand-free, open form of EcMBP shown in FIG. 1 (Sharff et al., Biochemistry, 31:10657-10663, 1992) to guide rational design of EcMBP-cpGFP fusions that would result in maltose-dependent GFP fluorescence.

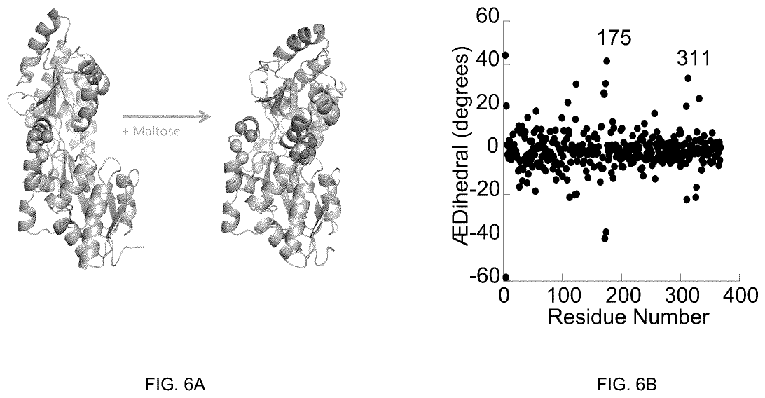

For (i), the change in dihedral angle (defined by the Ca atoms spanning four residues) was analyzed to identify maltose-dependent structural changes in sequentially adjacent residues (FIG. 6); this analysis showed that the Ca chain is “torqued” around residues 175 (ΔDihedral=+41°) and 311 (ΔDihedral=−22°) upon ligand binding. This sequential conformational change was predicted to be coupled to structural changes of an inserted cpGFP, resulting in maltose-dependent fluorescence for the fusion protein.

Previous studies using randomly digested and reassembled circularly permuted β-lactamase (cpB1a) and EcMBP showed maltose-dependent β-lactamase activity in proteins with insertions of cpB1a at EcMBP residues 165 and 317 (Guntas et al., Chem. Biol., 11:1483-1487, 2004; Guntas and Ostermeier, J. Mol. Biol., 336:263-273, 2004).

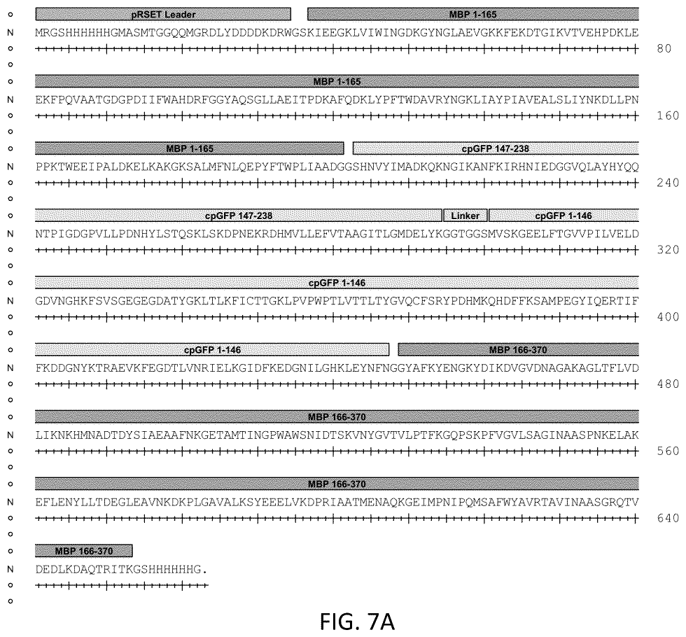

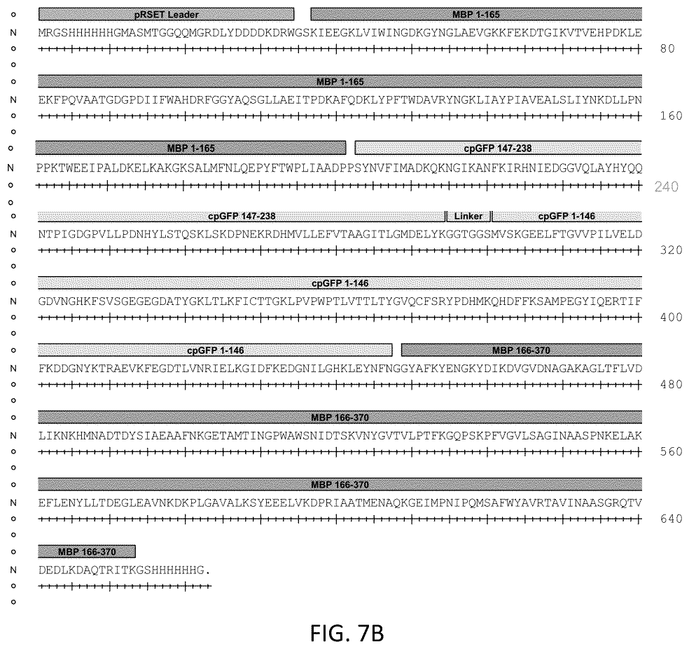

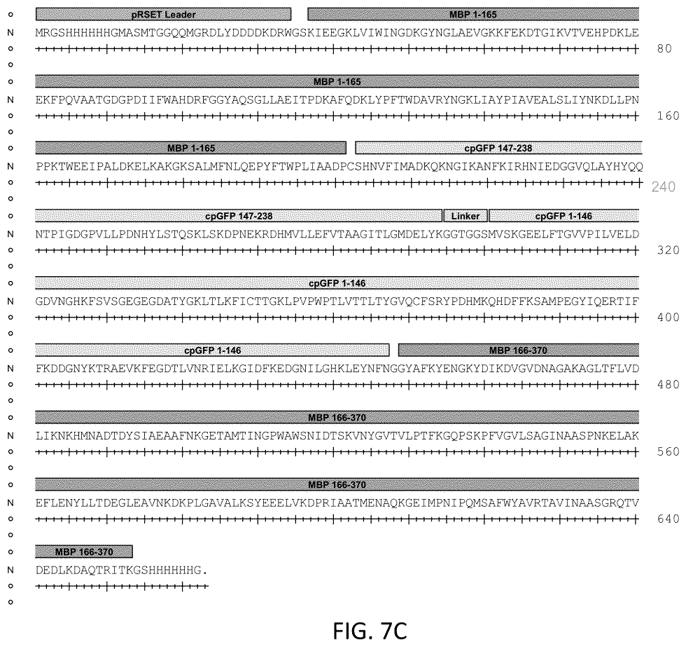

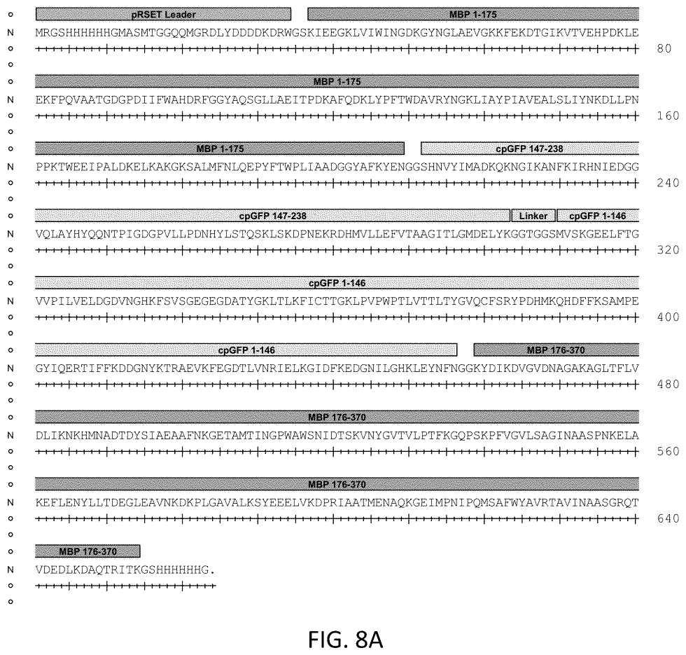

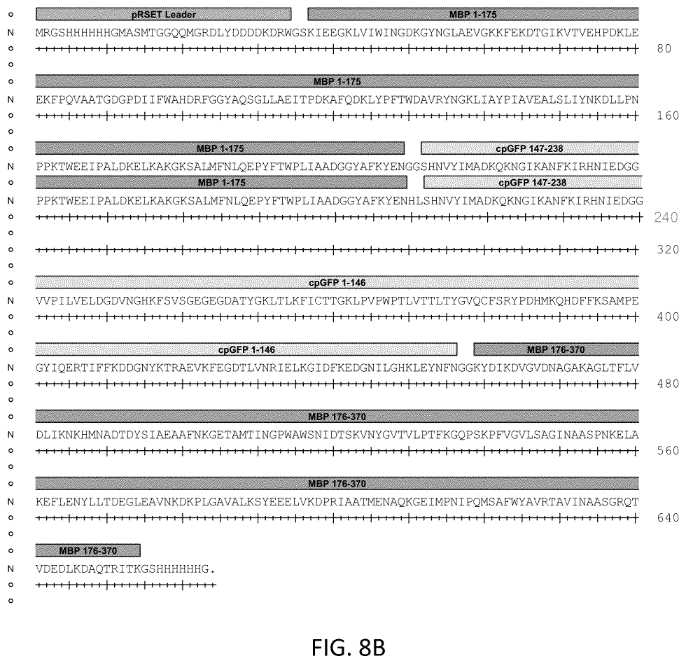

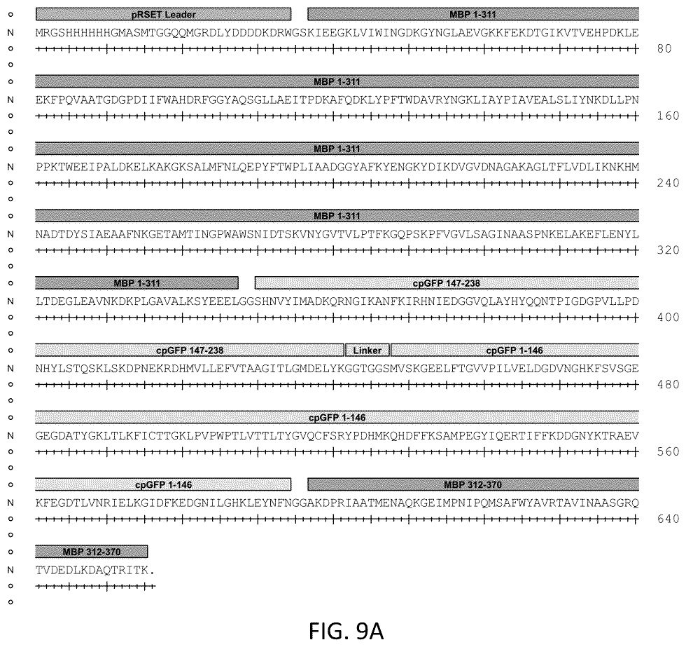

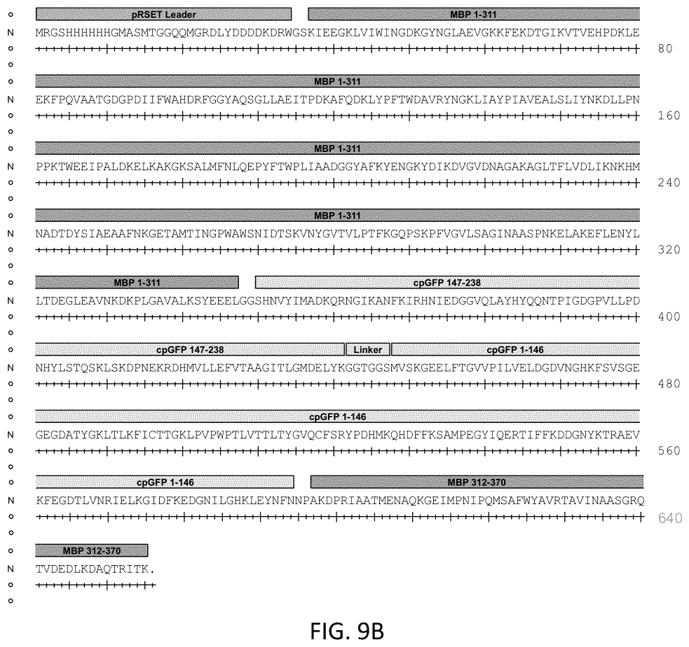

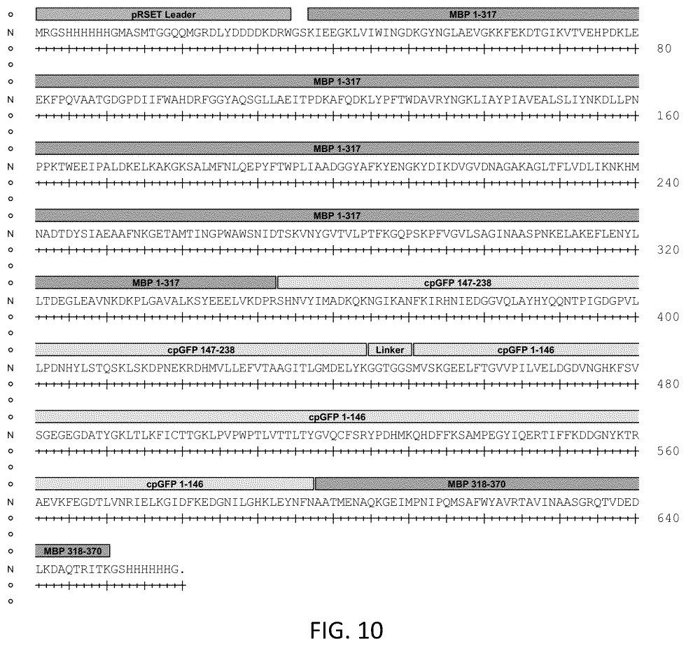

Since the ΔDihedral of EcMBP165 is +11° (moderate change) and EcMBP317 is +2° (no real change), four EcMBP-cpGFP templates were constructed by inserting cpGFP into EcMBP at sites 165, 175 (identified herein), 311 (identified herein), and 317 to test our predictive method and the interchangeability of cpB1a and cpGFP at sites identified from the EcMBP-cpB1a screen. These constructs were named MBP165-cpGFP, MBP175-cpGFP, MBP311-cpGFP, and MBP317-cpGFP (names were modified to encompass variants (e.g., with modified linker sequences). The cpGFP used is cpGFP146 described in Baird et al. (Proc. Natl. Acad. Sci., USA, 96:11241-11246, 1999). PCR assembly was used to construct fusion proteins with GlyGly-linkers between EcMBP and each terminus of cpGFP. The amino acid sequence of each construct is shown in FIGS. 6-9. The sequences of SEQ ID NOs:1-3 shown in FIGS. 7A-7C (i.e., MBP165-cpGFP) differ in the linker sequence between MBP 1-165 and cpGFP 147-238 (linker 1: see the line ending in amino acid 240)). The sequences of SEQ ID NOs: 4-5 shown in FIGS. 8A-8B (i.e., MBP175-cpGFP) differ in the sequence between MBP 1-175 and cpGFP 147-238 (linker 1: see the line ending in amino acid 240)). The sequences of SEQ ID NOs: 6-7 shown in FIGS. 9A-9B (i.e., MBP311-cpGFP) differ in the sequence between cpGFP 1-146 and MBP 312-370 (linker 2: see the line ending in amino acid 640)). Each construct includes 3 linkers: A linker between the C-terminus of the C-terminal portion of MBP and the N-terminus of cpGFP (i.e., linker 2), a linker between the N-terminus of cpGFP and C-terminus of the N-terminal portion of MBP, and a linker in cpGFP (i.e., linker 3).

### Example 1B: Linker Optimization

Libraries of variants of SEQ ID NOs: 1-8 were generated with randomized linkers by single-stranded uracil template mutagenesis (see Kunkel et al., Methods Enzymol., 204:125-139, 1991) using the primers listed below:

165 Linker 1 Primers:
(SEQ ID NO: 9)
PLIAADGxxNVYIM
(SEQ ID NO: 10)
PLIAADxxNVYIM
(SEQ ID NO 11)
PLIAADGGxxNVYIM
(SEQ ID NO: 12)
PLIAADGxPNVYIMG
(SEQ ID NO: 13)
PLIAADGIxNVYIMG
(SEQ ID NO: 14)
PLIAADPxSHNVYIM
(SEQ ID NO: 15)
PLIAADxPSHNVYIM
(SEQ ID NO: 16)
PLIAADxxSHNVYIM
(SEQ ID NO: 17)
PLIAADxxSHNVFIM
(SEQ ID NO: 18)
PLIAADPxSHNVFIM
(SEQ ID NO: 19)
PLIAADPxSYNVFIM
(SEQ ID NO: 20)
PLIAADxxSYNVFIM
(SEQ ID NO: 21)
PLIAADPxSYNVFIM
(SEQ ID NO: 22)
PLIAADxxSYNVFIM
(SEQ ID NO: 23)
PLIAADPxSxNVYIM
(SEQ ID NO: 24)
PLIAADPxSHxVYIM
(SEQ ID NO: 25)
PLIAADPxSHNxYIM
(SEQ ID NO: 26)
PLIAADPxSHNVxIM
165 Linker 2 Primers:
(SEQ ID NO: 27)
KLEYNFNxxYAFKYEN
(SEQ ID NO: 28)
KLEYNFNxYAFKYEN
(SEQ ID NO: 29)
KLEYNFNYAFKYEN
(SEQ ID NO: 30)
KLEYNFxYAFKYEN
(SEQ ID NO: 31)
KLEYNxxYAFKYEN
(SEQ ID NO: 32)
KLEYNWxYAFKYEN
(SEQ ID NO: 33)
KLEYNxKYAFKYEN
(SEQ ID NO: 34)
KLEYNFNPxYAFKYEN
(SEQ ID NO: 35)
KLEYNFNxPYAFKYEN
175 Linker 1 Primers:
(SEQ ID NO: 36)
AFKYENxxSHNVYIM
175 Linker 2 Primers:
(SEQ ID NO: 37)
KLEYNFNxxKYDIKDV
311 Linker 1 Primers:
(SEQ ID NO: 38)
KSYEELxxSHNVYIM
(SEQ ID NO: 39)
KSYEELPxSHNVYIM
(SEQ ID NO: 40)
KSYEELxPSHNVYIM
311 Linker 2 Primers:
(SEQ ID NO: 41)
KLEYNFNxxAKDPRIA
(SEQ ID NO: 42)
KLEYNFNPxAKDPRIA
(SEQ ID NO: 43)
KLEYNFNxPAKDPRIA
317 Linker 1 Primers:
(SEQ ID NO: 44)
ELAKDPRxSHNVYIM
(SEQ ID NO: 45)
ELAKDPRxxSHNVYIM
(SEQ ID NO: 46)
ELAKDPRxxxSHNVYIM
317 Linker 2 Primers:
(SEQ ID NO: 47)
KLEYNFNxAATMENA
(SEQ ID NO: 48)
KLEYNFNxxAATMENA
(SEQ ID NO: 49)
KLEYNFNxxxAATMENA

Where “x” indicates that a degenerate primer (with DNA sequence “NNS”) was used to encode all 20 possible amino acids.

About 400 variants were screened in semi-high-throughput fashion, measuring fluorescence intensity of clarified cell lysate in the absence and presence of 10 mM maltose.

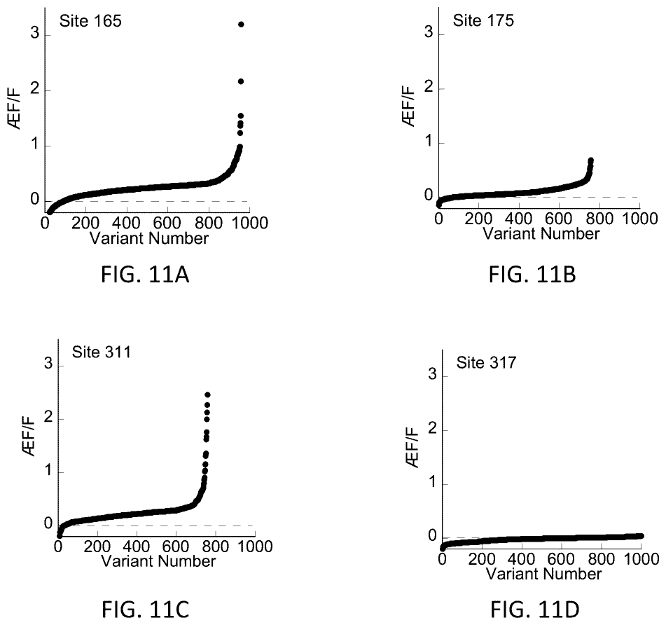

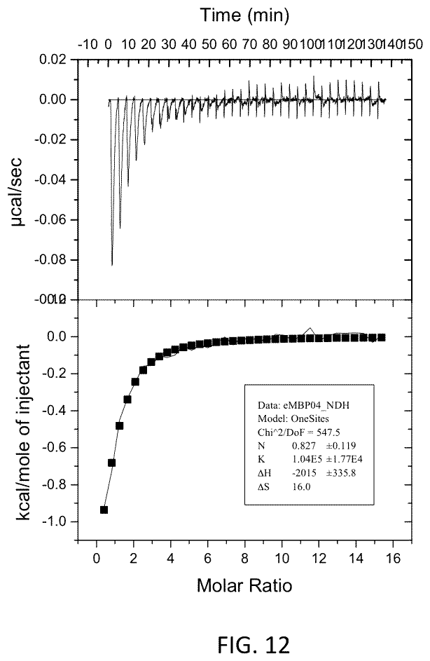

Insertion of cpGFP as MBP317, a site previously reported for cpB1a, did not show maltose-dependent fluorescence (FIG. 11) even though the framework protein still bound maltose, as determined by isothermal titration calorimetry (FIG. 12). These data demonstrate that identification of insertion sites by a method other than insertion of cpGFP (such as insertion of cpB1a) is not sufficient to identify sites that transduce ligand binding to changes in fluorescence intensity

Insertion of cpGFP at residue 165 of EcMBP (EcMBP165-cpGFP), another position reported in cpB1a studies (Guntas and Ostermeier, supra) with -GlyGly-linkers flanking the cpGFP resulted in a protein in which fluorescence increased 20% (ΔF/F=0.2) upon addition of saturating maltose.

Screening a fully-degenerate, length-two library (“XX”) at either the EcMBP-cpGFP linker (linker 1) or the cpGFP-EcMBP linker (linker 2) yielded proteins with maltose-dependent fluorescent increases >300% or decreases >50% (FIG. 11). Many of the variants with increased ΔF/F values had linkers containing proline(s). Subsequent libraries constructed from oligonucleotides encoding XP or PX and randomization of the residues in GFP from residue 146 to 150 were screened, yielding a final variant with: a two-proline EcMBP-cpGFP linker, a two-glycine cpGFP-EcMBP linker, GFP-H148Y, and GFP-Y151F. This variant, called “EcMBP165-cpGFP.PPYF” (abbreviated PPYF (SEQ ID NO:2)) has a ΔF/F=2.5, a Kd for maltose of 3 μM. Screens also identified variant EcMBP311-cpGFP.L2-NP (-AsnPro- at linker 2 (SEQ ID NO:7)), which has a ΔF/F of 1.0 and a Kd for maltose of 2 μM. This variant has an inferior maltose-dependent fluorescence increase than PPYF, but demonstrates generality of the cpFP insertion method.

EcMBP175-cpGFP was also screened with XX linkers, and a few variants with ΔF/F≈1 were identified (FIG. 11). One mutant, with the first linker encoding HL (EcMBP175-cpGFP.L1-HL (SEQ ID NO:5)), has a ΔF/F=0.5 and a Kd for maltose of 1.3 μM.

These data support that choice of insertion site by structural analysis is preferable to random insertion.

### Example 1C: Modifying Ligand Binding and/or Fluorescent Properties of Sensors

One objective in the development of generic biosensors is for the framework to permit independent optimization of binding and signaling properties. Analysis of whether biosensors herein permit such optimization was tested using the high-SNR sensor PPYF, by: (i) rationally altering maltose-binding affinity; (ii) changing the ligand-binding specificity from maltose to sucrose, and (iii) creating a family of sensors in multiple colors.

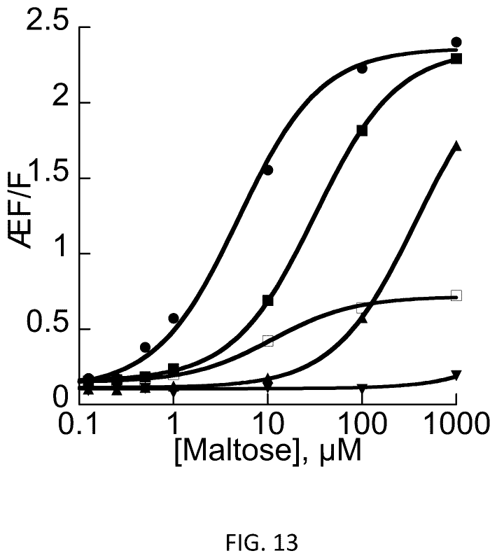

As a first step, the impacts of mutations of three tryptophan side-chains in the maltose-binding pocket (W230, W62, and W340) were tested. These sites have previously been shown to lower the affinity of EcMBP for maltose by one, two, or three orders of magnitude, respectively, when mutated to alanine (Martineau et al., J. Mol. Biol., 214:337-352, 1990). A mutation to the hinge region, I329W, was also made to PPYF, as this has been shown to increase maltose affinity by about 2-fold in both wild-type EcMBP (Marvin and Hellinga, Nat. Struc. Biol., 8:795-798, 2001) and in the EcMBP-cpB1a switches (Guntas et al., Chem. Biol., 11:1483-1487, 2004; Kim and Ostermeier, Arch. Biochem. Biophys., 446:44-51, 2006). As shown in FIG. 13, for the PPYF sensor, the three tryptophan-to-alanine binding-pocket mutations behaved as expected, lowering affinity by between one and three orders of magnitude. In contrast, the I329W mutation did not increase affinity as expected, but rather decreased it. ΔF/F also decreased. This data suggests that the mechanism of fluorescence change in this sensor is dependent on subtle interactions between EcMBP and cpGFP that are linked to the I329W mutation.

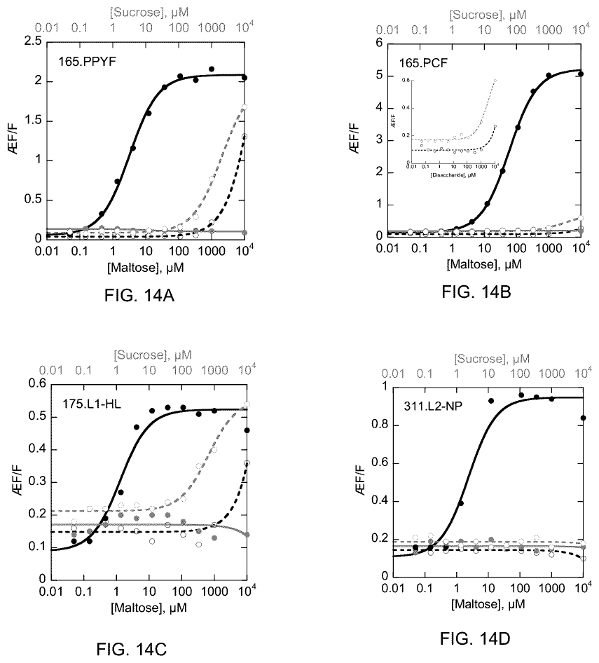

As an alternative test for changing the ligand-binding specificity of the sensor while preserving fluorescence signaling, “5-7” mutations (D14L, K15F, W62Y, E111Y), previously shown to confer EcMBP with an affinity for sucrose (Guntas and Mansell, Proc. Natl. Acad. Sci., 102:11224-11229, 2005), were made to PPYF. As shown in FIG. 14A, the mutations conferred to the sensor about 2 mM affinity for sucrose and ˜3 mM affinity for maltose. To address a discrepancy between expected (micromolar) and observed (millimolar) affinity for disaccharides, the 5-7 mutations were made to sensors with cpGFP inserted at different positions in EcMBP, and with different linker compositions. In the context of EcMBP165-cpGFP.PCF, the 5-7 mutations conferred very low (but observable) binding preference for sucrose over maltose (FIG. 14B). The trend of higher (but still weak) affinity for sucrose (˜0.6 mM) over maltose (˜6 mM) continued when the 5-7 mutations are made in the context of EcMBP175-cpGFP.L1-HL (FIG. 14C). In the context of EcMBP311-cpGFP.L2-NP, the 5-7 mutations appeared to eliminate all binding (FIG. 14D). The preference for sucrose over maltose of the 5-7 variants of the sensors is consistent with the binding properties of the 5-7 variants of EcMBP alone and EcMBP-cpB1a (Guntas and Mansell, Proc. Natl. Acad. Sci., 102:11224-11229, 2005). The lower affinity for both ligands of the 5-7 variants of the sensors may be the consequence of the inserted cpGFP shifting the open and closed equilibrium.

These data suggest that ligand binding and fluorescent properties of biosensors can be independently modified.

### Example 1D: Modifying Sensor Color

The color of GFP can be altered by changing the amino acids that either comprise or interact with the chromophore (see Shaner et al., J. Cell. Sci. 120:4247-4260, 2007, for a review).

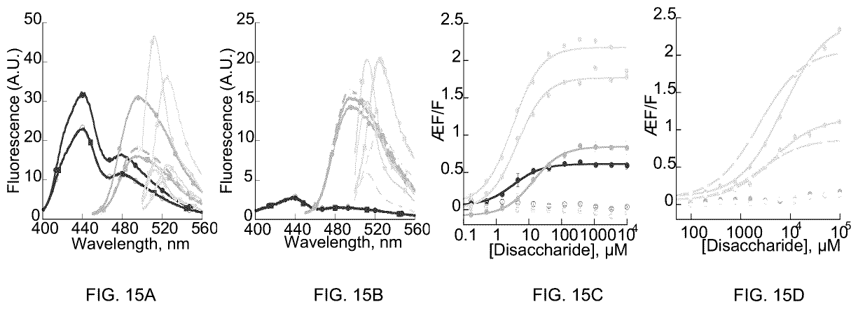

Using PPYF as a template, mutations Y66W (to yield a cyan variant, “cpCFP”), L64F+T65G+V68L+T203Y (yellow, “cpYFP”), and Y66H (blue, “cpBFP”) mutations were made (see Cubitt et al., Trends Biochem., 20:448-455, 1995, for exemplary methods). As shown in FIG. 15, the variants exhibit fluorescence emission spectra consistent with their respective intended designs.

The ΔF/F of the color variants in response to maltose is different (in each case inferior) from the ΔF/F of 2.5 observed in PPYF-green. The EcMBP165-cpYFP.PPYF sensor, which has the same covalent chromophore structure as PPYF, has the greatest ΔF/F of the three spectral variants (FIG. 15A). EcMBP165-cpCFP.PPYF has a lower ΔF/F than the green and yellow variants, but by incorporating previously identified mutations, (L1-PC+GFP-Y151F; the resulting protein is called EcMBP165.cpCFP.PCF), a variant with ΔF/F=0.8 was obtained (FIG. 15A).

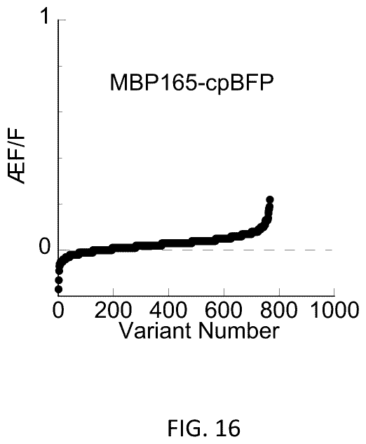

The EcMBP165-cpBFP.PPYF variant, while dimly fluorescent, is not a sensor, and a screen of 800 linker variants failed to produce any variant with ΔF/F >0.2 (FIG. 16).

Since EcMBP165-cpBFP.PPYF was very dim, Azurite mutations T65S+V150I+V224R were included to increase brightness and stability, and make EcMBP165-cpAzurite a good template for linker screening. Using oligonucleotides encoding XX amino acid linkers, a variant was obtained, EcMBP165-cpAzurite.L2-FE, that had ΔF/F=0.8 (FIG. 15).

### Example 1E: Modifying Sensor Color and Ligand Specificity/Affinity

The four sucrose-binding “5-7” mutations described above that conferred weak sucrose affinity in the green sensor (EcMBP165-cpGFP.PPYF) were converted to blue, cyan, and yellow maltose sensors (EcMBP165-cpAzurite.L2-FE, EcMBP165-cpCFP.PCF, and EcMBP165-cpYFP.PPYF). The green and yellow sensors showed increased fluorescence upon addition of 10 mM sucrose, but the cyan and blue proteins did not (FIG. 15A). Like the green variant, the yellow variant had no detectable sucrose affinity with the wild type binding pocket (FIG. 15C) and millimolar affinity for both sugars, with preference for sucrose over maltose (FIG. 15D).

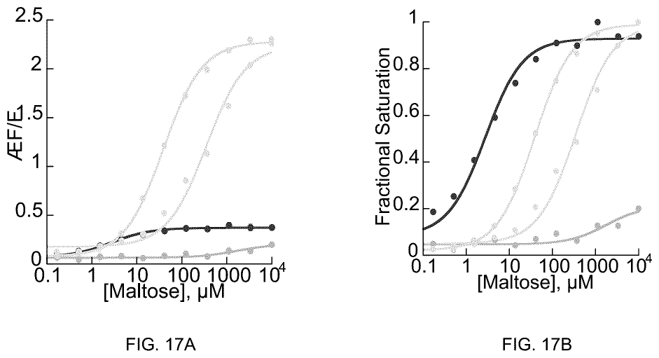

As seen in FIG. 17, as maltose concentration increased, the blue sensor increased in fluorescence first (Kd ˜2.7 μM), then the green (Kd ˜40 μM), then the yellow (Kd ˜350 μM), and at high maltose concentrations, the cyan variant began to increase its fluorescence (Kd ˜1.7 mM).

### Example 1F: Imaging Bacteria

The ultimate value of genetically encoded fluorescent sensors is in their utility for observing analyte flux in living cells and organisms. In a simple proof-of-principle experiment, Escherichia coli expressing PPYF or PPYF.T203V (see “Second-generation maltose sensors” below) were imaged in the green fluorescence channel in the absence of maltose, and then re-imaged after addition of saturating maltose to the media.

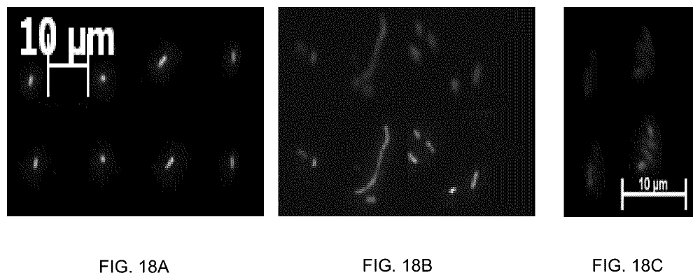

As shown in FIG. 18, bacteria expressing the sensors clearly became brighter, while control bacteria expressing EGFP appeared unchanged. Increased fluorescence was quantified by measuring the peak (gray-value) pixel intensity of each bacterium. Those expressing PPYF undergo an approximate doubling of fluorescence (bacterium-averaged ΔF/F=1.1±0.4), those expressing PPYF.T203V have slightly increased ΔF/F (ΔF/F=1.29±0.2), while those expressing EGFP have no change in fluorescence (ΔF/F=−0.01±0.05).

### Example 1F: 2-Photon Imaging of Mammalian Cells

Multi-photon microscopy opened new frontiers for in vivo fluorescence imaging, in particular for neuronal activity visualization through the use of genetically encoded calcium indicators (Tian et al., Nat. Methods, 3:281-286, 2009; Denk et al., Science, 248:73-76, 1990; Denk and Svoboda, Neuron, 18:351-357, 1997).

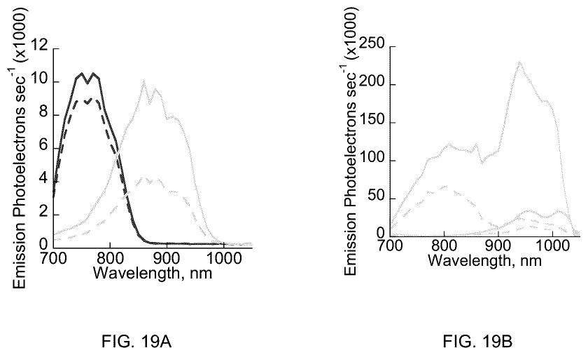

To demonstrate that the maltose sensors described herein have the potential to be used for 2-photon imaging, fluorescence excitation spectra were collected. As shown in FIG. 19, with a 535 nm bandpass emission filter (50 nm bp), EcMBP165-cpGFP.PPYF showed a 10-fold maltose-dependent increase in fluorescence when excited at 940 nm. All four spectral variants showed a significant maltose-dependent increase in 2-photon fluorescence.

### Example 1G: Sub-Cloning Maltose Sensors

EcMBP165-cpGFP.PPYF.T203V (see “Second-generation maltose sensors” below) were cloned into a modified version of the pDisplay vector (Invitrogen) for extracellular display on the surface of transiently transfected human embryonic kidney (HEK293) cells.

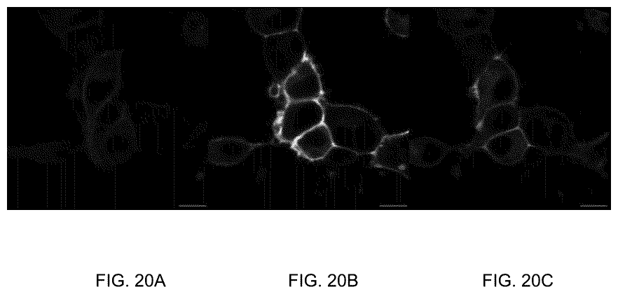

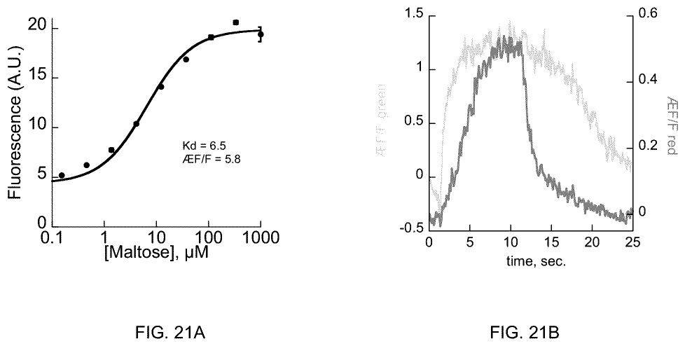

As shown in FIG. 20, the sensor localized to the plasma membrane and increased in brightness in a concentration-dependent manner when perfused with buffers of varying maltose concentration. The ΔF/F is 5.8-fold, very close to that of the soluble protein produced in E. coli, with the mid-point of the maltose-dependent fluorescence increase being 6.5 μM (FIG. 21A), very similar to the affinity determined on purified protein (5 Furthermore, the surface displayed sensor responded rapidly to a pulse of 1 mM maltose (FIG. 21A), indicating that the time course for its action is useful for transient events.

### Example 1H: Crystal Structure Analysis of Maltose Sensors

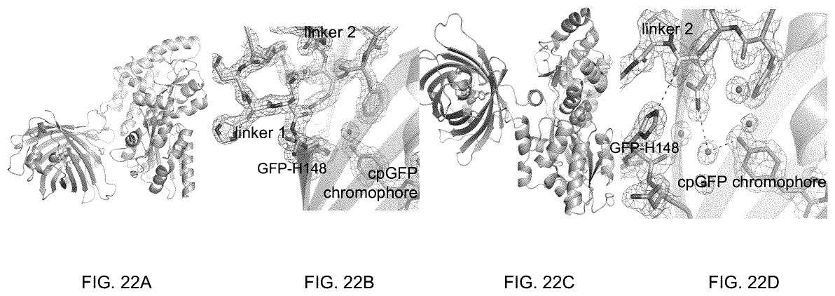

High-resolution structures of several of the maltose sensors described above were generated. Crystallization trials were performed with EcMBP165-cpGFP.PPYF, EcMBP175-cpGFP.L1-HL, and EcMBP311-cpGFP.L2-NP in the presence and absence of excess maltose, from which both EcMBP175-cpGFP.L1-HL and EcMBP311-cpGFP.L2-NP crystallized in the presence of maltose. X-ray structures were solved to 1.9 and 2.0 Å resolution, respectively, by molecular replacement (FIGS. 22A-22C).

The structures of the cpGFP and EcMBP domains of the sensors are superimposable with published crystal structures of cpGFP (from GCaMP2; RMSD=0.36 and 0.38 Å, respectively, for comparing 221 common Ca atoms) and EcMBP-maltose (RMSD=0.43 and 0.37 Å, 370 Ca). The structure of EcMBP is largely unperturbed by insertion of the cpGFP domain; only residues around the 175 and 311 insertion sites showed any significant displacement.

GFP-H148, which H-bonds the GFP chromophore in the structure of native GFP, also directly H-bonded to the chromophore in the EcMBP175-cpGFP.L1-HL-maltose structure (FIG. 22B), although a different rotamer was observed. In the EcMBP311-cpGFP.L2-NP-maltose structure, GFP-H148 is pulled away from the chromophore and is largely replaced by the Asn from linker 2, which makes H-bond interactions to both strand 8 of the GFP barrel and the chromophore phenolate oxygen (through a water molecule, FIG. 22D). GFP-H148, meanwhile, seemed to stabilize the conformation of linker 2 of EcMBP311-cpGFP.L2-NP by H-bonding the backbone carbonyl of the linker 2 Asn. There is some solvent access to the cpGFP chromophore through the hole in the GFP barrel created by circular permutation, although the inter-domain linkers block much of the opening in both structures. Relatively few contacts are made between the cpGFP and EcMBP domains.

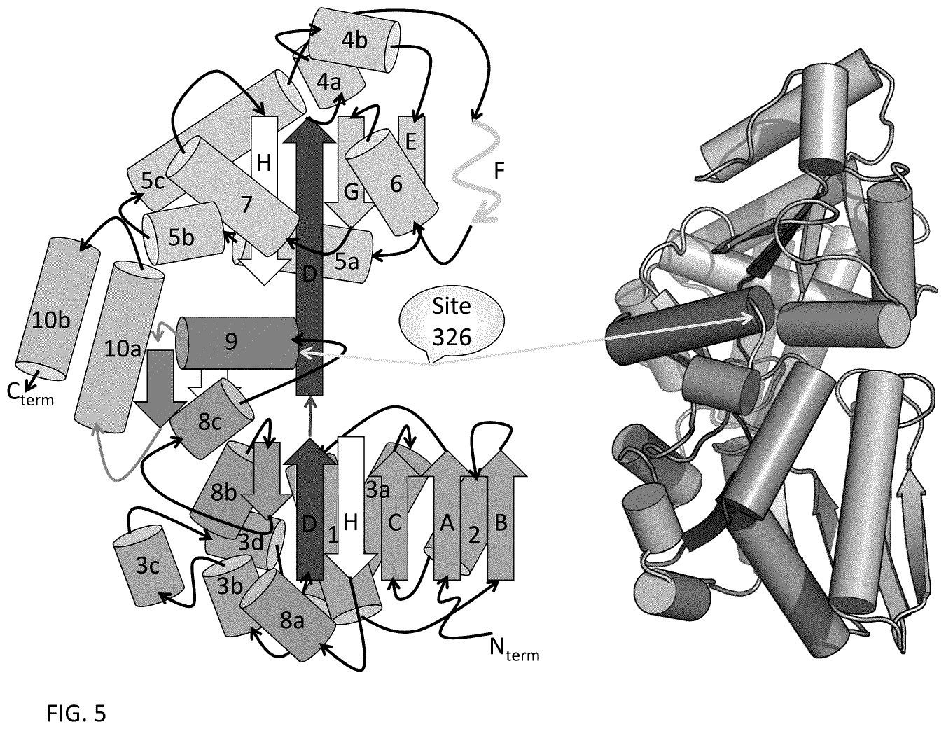

Based on the structures of two maltose-bound sensors, the sensing mechanism likely involves a shift in the relative position of linker 1 and linker 2 induced by the conformational change in the EcMBP domain associated with maltose binding (FIG. 5). The register shift of interactions between the two linkers could alter the proximity of linker 2 and nearby side-chains to the cpGFP chromophore and change the water structure in the cpGFP opening, leading to a shift in the chromophore protonation equilibrium. This might explain why rigid proline is preferred in either linker, since conformational changes upon ligand binding might be better propagated through the rigid linkers to the cpGFP chromophore environment.

### Example 1I: Generation of Second-Generation Maltose Sensors

In an attempt to increase brightness and ΔF/F of GCaMP, the local environment of the chromophore was altered by randomizing residues within cpGFP, and screening for improved variants (Tian et al., nat. Methods, 6:875-881, 2009).

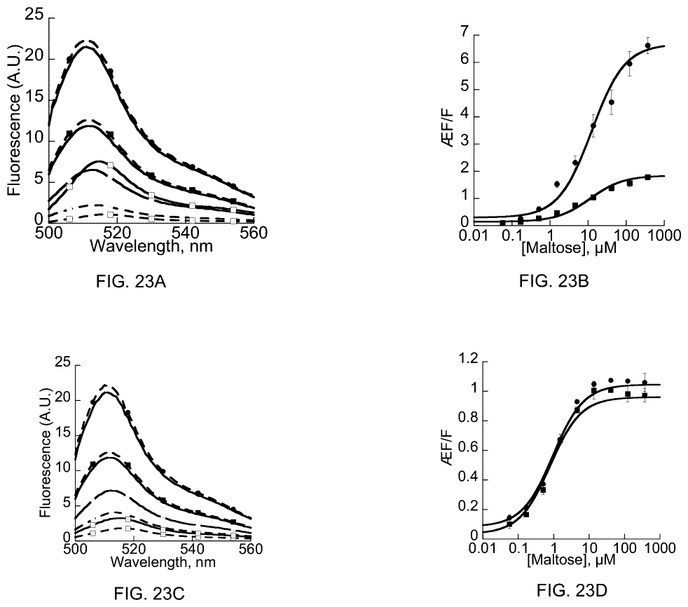

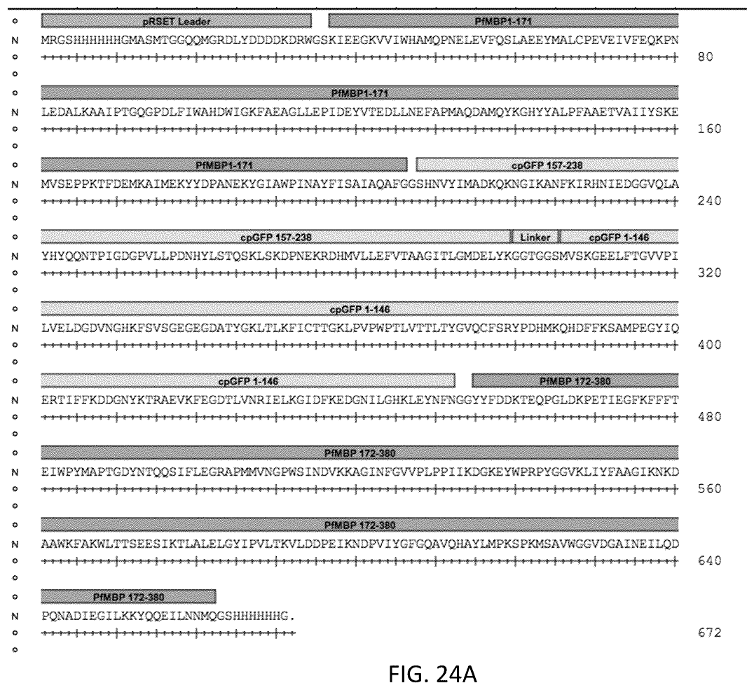

As shown in FIG. 23, in the context of EcMBP165-cpGFP.PPYF, the T203V mutation decreases the fluorescence emission of the apo-state by half (FIG. 23A), while saturated fluorescence and affinity are unchanged (FIG. 23B), increasing ΔF/F to 6.5. In the maltose-saturated state, PPYF itself has about a quarter the brightness of EGFP, and half the brightness of cpGFP.

In the context of EcMBP311-cpGFP.L2-NP, the T203V mutation decreases the brightness of both the apo-state and the saturated-state equally, resulting in no significant change in ΔF/F (FIGS. 23C and D).

These results indicate that the benefits of the T203V mutation are not universally transferable, and that cpGFP-based fluorescent sensors need to be optimized individually.

### Example 2: Maltotriose Indicators

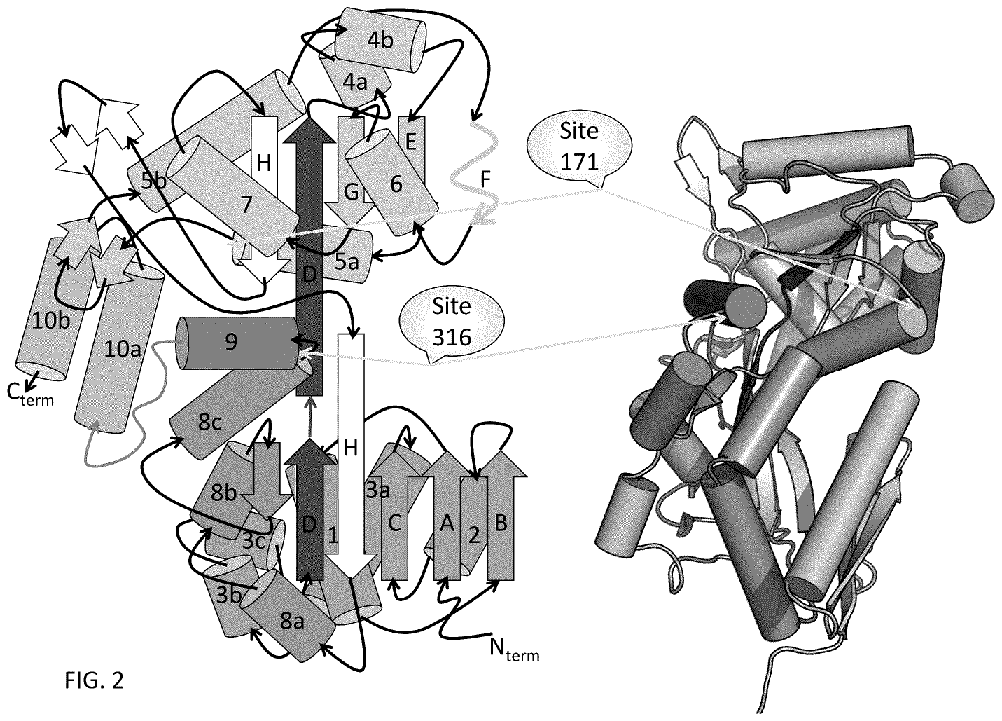

Genetically encoded maltotriose indicators were created using Pyrococcus furiosus maltotriose binding protein. As described below, only the structure of the ligand-bound state P. furiosus maltotriose binding protein (PfMBP) is available. As shown in FIGS. 1 and 2, PfMBP is homologous to EcMBP (compare FIGS. 1 and 2). Two sensors were made, PfMBP171 and PfMBP316, the insertion points for which were selected based on homology to EcMBP165 and EcMBP311, respectively. Linkers were optimized. PfMBP sensors have a ΔF/F of ˜1.2.

Pyrococcus furiosus is a thermophilic organism. Proteins from thermophiles have been shown to be more amenable to mutation than those from mesophiles (Bloom et al., Proc. Natl. Acad. Sci., 103:5869-5874, 2006). As an alternative to developing new sensors by inserting cpGFP into PBPs, it should also be possible to generate new sensors by changing the ligand-binding specificity of an existing PBP-based sensor.

It has previously been shown that the binding sites of PBPs can be reengineered to accommodate novel ligands (Looger et al., Nature, 423:185-190, 2003). However, those re-design efforts used framework proteins from mesophiles and suffered from poor stability. We hypothesized that PfMBP, which is intrinsically more stable than EcMBP, is more tolerant of mutations. To test this hypothesis, we characterized and compared the stability of PfMBP to EcMBP, PfMBP-cpGFP sensors to EcMBP-cpGFP sensors, PfMBP binding site mutants to EcMBP binding site mutants, and PfMBP-cpGFP sensor binding site mutants to EcMBP-cpGFP sensor binding site mutants. Conclusively, the PfMBP variants were more stable than the EcMBP variants. Finally, we demonstrate that the increased thermo-stability of the PfMBP-cpGFP sensors is useful for the measurement of maltotriose at temperatures as high at 60° C., whereas the EcMBP-cpGFP sensors are only useful for the measurement of maltose at temperatures as high as 40° C.

### Example 2A: Identification of cpGFP Insertion Sites in PfMBP

The ligand-bound (closed) structure of PfMBP is available (Evdokimov et al., J. Mol. Biol., 305:891-904, 2001), but the unbound structure is not. Accordingly, insertion sites for the PfMBP-cpGFP sensors were identified by homology to EcMBP.

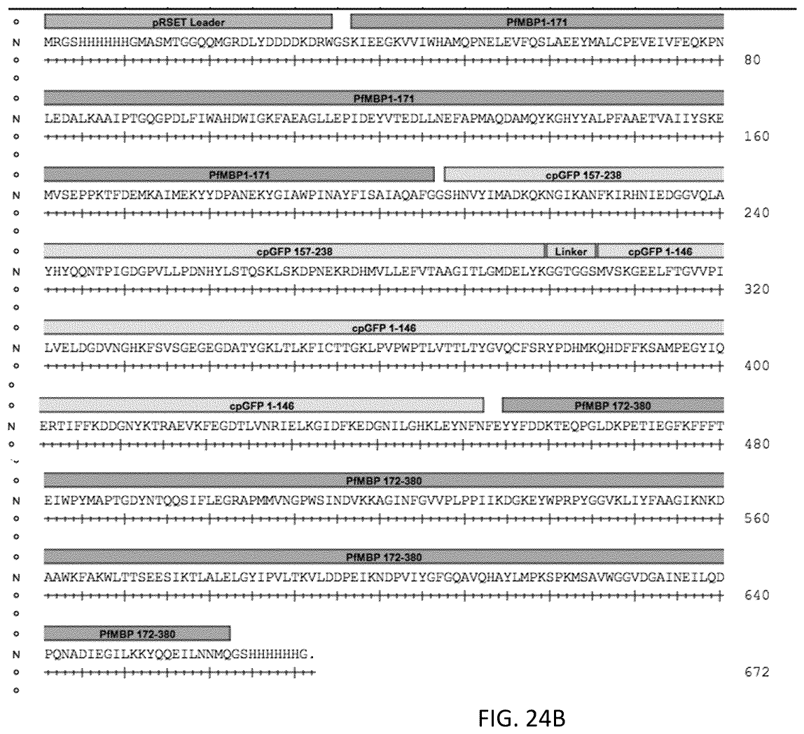

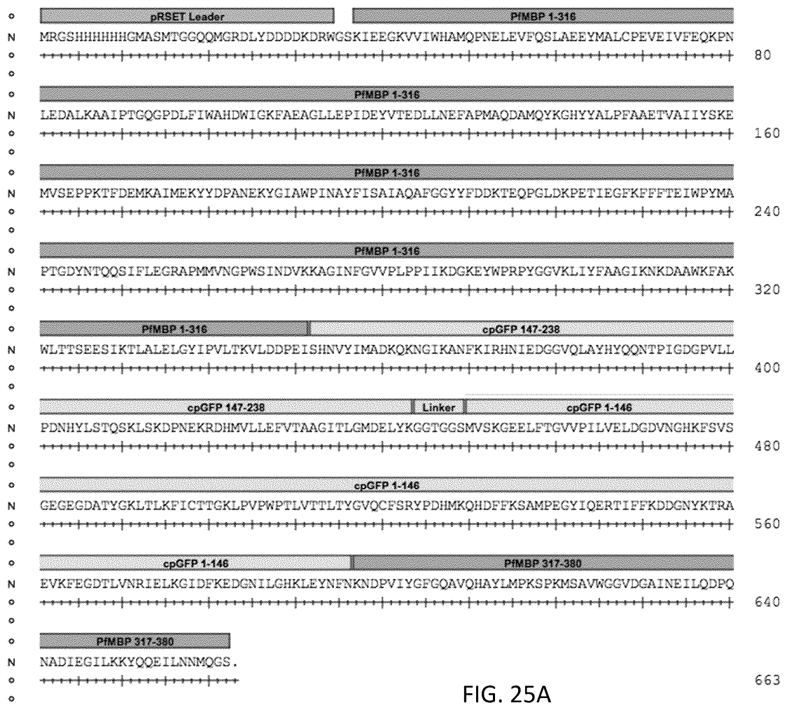

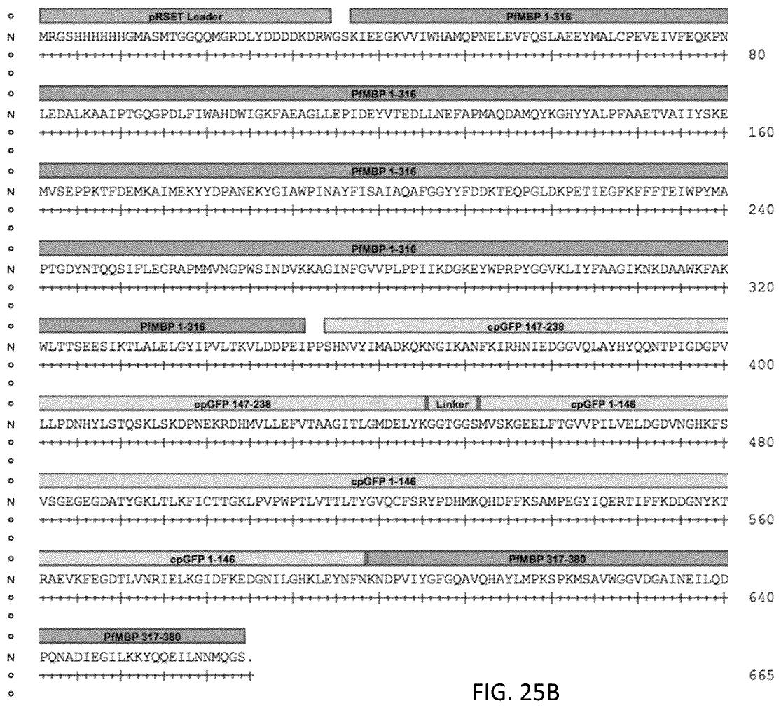

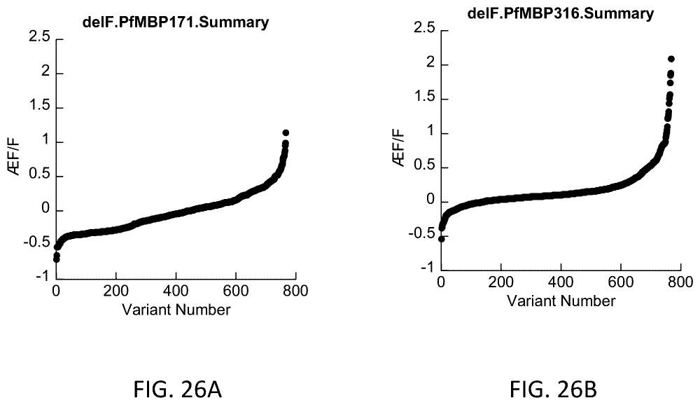

Sites were selected based on the structural similarities between PfMBP and EcMBP. Two sites were selected. One of these sites is EcMBP311, which is homologous to PfMBP316. This site is at juncture between the end of the cluster of helices (Helices 8a, 8b, 8c) and the start of the “equatorial” spanning helix (Helix 9). Another site that was made into a sensor in EcMBP was EcMBP165, which is homologous to PfMBP171. cpGFP was inserted into PfMBP at each of these sites. The sequences of the resulting constructs, PfMBP171-cpGFP and PfMBP316-cpGFP, are shown in FIGS. 24 and 25, respectively.

### Example 2B: Linker Optimization

Libraries of variants of SEQ ID NOs: 50-53 were generated with randomized linkers by single-stranded uracil template mutagenesis using the primers listed below:

175 Linker 1 Primers:
(SEQ ID NO: 54)
AIAQAFxxSHNVYIMA
(SEQ ID NO: 55)
AIAQAFPxSHNVYIMA
171 Linker 2 Primers:
(SEQ ID NO: 56)
KLEYNFNxxYYFDDKTE
316 Linker1 Primers
(SEQ ID NO: 57)
VLDDPExxHNVYIM
(SEQ ID NO: 58)
VLDDPEIxxSHNVYIM
316 Linker2 Primers
(SEQ ID NO: 59)
KLEYNFxxNDPVIY
(SEQ ID NO: 60)
KLEYNFNxPKNDPVIY
(SEQ ID NO: 61)
KLEYNFNPxKNDPVIY

Where “x” indicates that a degenerate primer (with DNA sequence “NNS”) was used to encode all 20 possible amino acids.

Several thousand variants were screened in semi-high-throughput fashion, measuring fluorescence intensity of clarified cell lysate in the absence and presence of 1 mM maltotriose.

Screening a fully-degenerate, length-two library (“XX”) at either the PfMBP171-cpGFP linker (linker 1) or the cpGFP-PfMBP linker (linker 2) yielded proteins with maltotriose-dependent fluorescent increases >100% or decreases >20% (FIG. 26A). A variant from this group with a GlyGly PfMBP-cpGFP linker and a PheGlu cpGFP-PfMBP linker was selected for further characterization. This variant, called “PfMBP171-cpGFP.L2FE” has a ΔF/F=1.2, a Kd for maltotriose of <1 μM.

Screening a fully-degenerate, length-two library (“XX”) at either the PfMBP316-cpGFP linker (linker 1) or the cpGFP-PfMBP linker (linker 2) also yielded proteins with maltotriose-dependent fluorescent increases >100% or decreases >20% (FIG. 26B). A variant from this group with a GlyGly PfMBP-cpGFP linker and a PheGlu cpGFP-PfMBP linker was selected for further characterization. This variant, called “PfMBP316-cpGFP.L1-NP” has a ΔF/F=1.2, a Kd for maltotriose of 40 μM.

These data support that structurally homologous frameworks can be compared to identify insertion sites for cpGFP.

### Example 2C: Characterization of the Thermostability of the PfMBP and PfMBP-cpGFP Compared to EcMBP and EcMBP-cpGFP

Thermal stability of PfMBP171-cpGFP.L2FE was measured using circular-dichroism (CD) and compared to the original EcMBP and PfMBP binding proteins, along with cpGFP. Following the changes by means of CD allowed determination of whether different transitions happened in alpha, beta, or both kinds of structures.

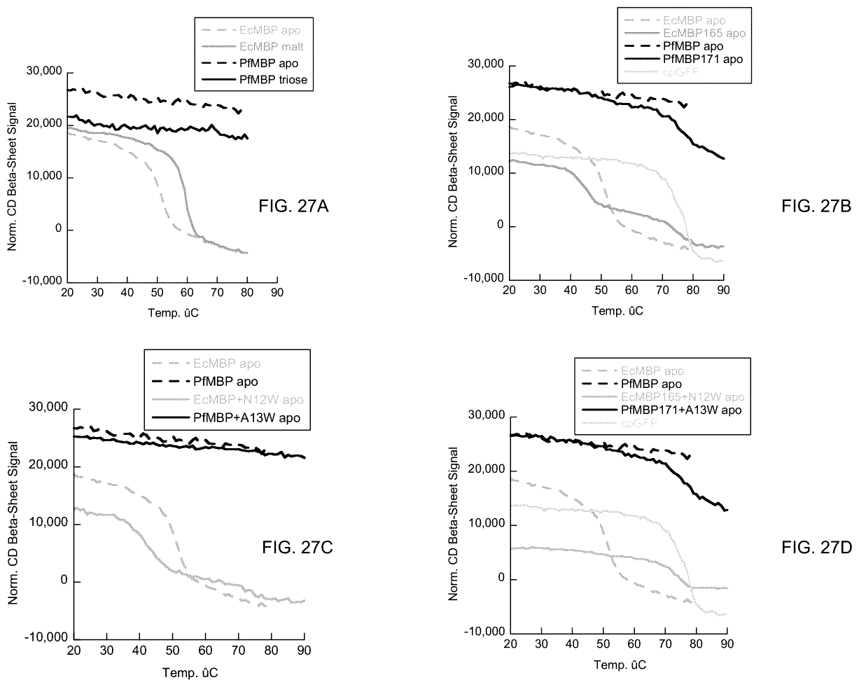

Given that cpGFP is a beta barrel, strong transitions in the beta signal alone were associated with changes in this kind of structure. In the same way, transitions in both kinds of signals were associated with the binding protein structure. As shown in FIG. 27A, PfMBP is significantly more thermo-stable than EcMBP. In fact, while EcMBP denatured at about 50° C., PfMBP did not denature at temperatures less than 80° C. Also, the addition of maltose to EcMBP stabilized the protein by about 10° C.

As shown in FIG. 27B, the stability of the EcMBP component of the EcMBP165-cpGFP.PPYF sensor decreased from 50° C. to 45° C. with insertion of cpGFP, while the intrinsic stability of cpGFP in the sensor remained unchanged. There was little change in the stability of the PfMBP component of the PfMBP171-cpGFP.L2FE sensor with insertion of cpGFP (FIG. 27B). Moreover, PfMBP seemed to exert a small stabilizing effect over the inserted cpGFP, as shown by the change in the steepness and melting point of the curve of the soluble form and the PfMBP171-cpGFP.L2FE sensor. All the associations made between transitions and domain unfolding were supported by CD spectra taken at the beginning and the end of each temperature ramp.

Analysis of whether the PfMBP scaffold was more tolerant of mutation than the EcMBP scaffold was also performed. Proof-of-principle mutations were made to the ligand-binding sites of EcMBP and PfMBP, and their respective sensors. In EcMBP, Asn12 was mutated to Trp to result in steric clashes with the surrounding residues, and backbone, of the binding pocket. The homologous mutation in PfMBP is Ala13Trp, which would be expected to have the same effect.

As shown in FIG. 27C, N12W decreased the Tm of EcMBP from 50° C. to 40° C., while the corresponding mutation in PfMBP, A13W, had no noticeable effect. This data confirms that the thermophilic protein is more tolerant of mutations to the binding site. Furthermore, in the context of the sensors, the N12W mutation to EcMBP165-cpGFP.PPYF completely destabilized the binding protein component of the sensor (FIG. 27D), while the A13W mutation in PfMBP171-cpGFP.L2FE had no effect on stability (FIG. 27D).

### Example 2D: Tolerance of PfMBP Sensor to Increased Temperature

Fluorescence of the protein in the apo and ligand-bound states at was measured at different temperatures.

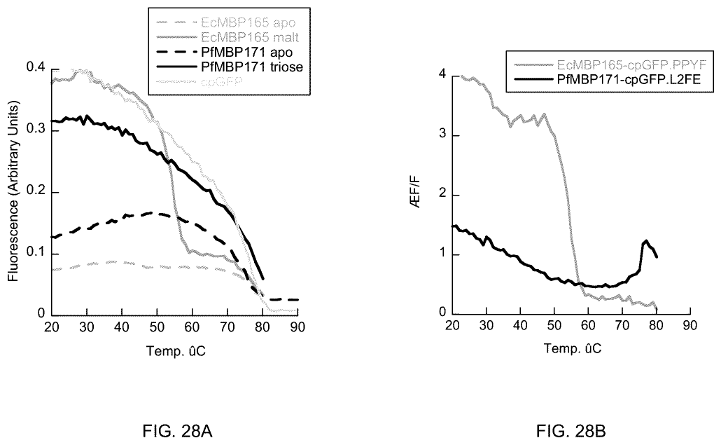

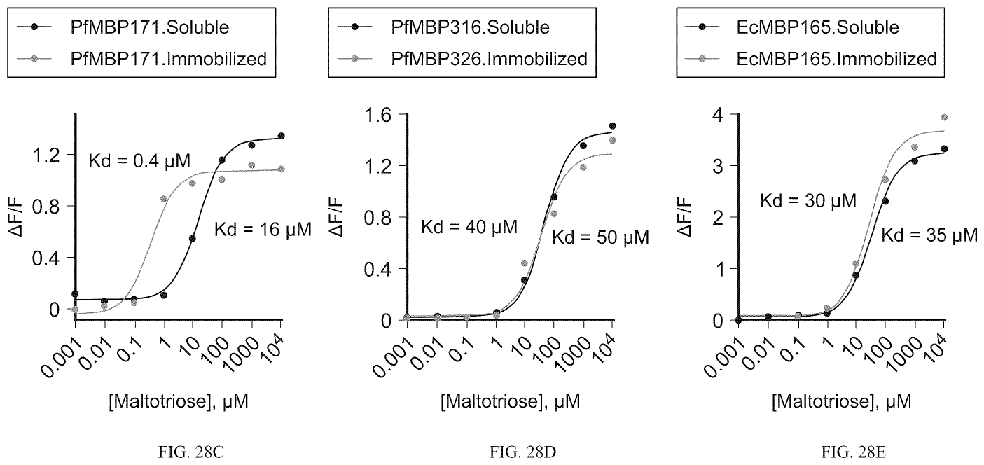

As shown in FIG. 28A, fluorescence of the EcMBP165-cpGFP.PPYF sensor in the bound state was higher than it is in the apo-state at lower temperatures, by about 4-fold. However, at around 55° C. (the unfolding transition of the EcMBP component) the fluorescence of the EcMBP165-cpGFP.PPYF sensor dropped precipitously. As a result, EcMBP165-cpGFP.PPYF is unsuitable for detection of maltose at temperatures greater than 50° C. (FIG. 28B). In contrast, PfMBP171-cpGFP.L2FE retained its maltotriose binding capabilities at high temperatures (FIGS. 28A and 28B), and is limited only by the intrinsic fluorescence of the cpGFP component, which decays at about 80° C. (FIG. 28A).

### Example 2E: Measurement of Maltodextrins in Hot Liquids

To demonstrate that the soluble and immobilized sensors function similarly, PfMBP171-cpGFP.L2FE, PfMBP316-cpGFPL1XXX, and EcMBP165-cpGFP.PPYF.T203V were immobilized via their N-terminal poly-histidine tags on to the surface of Ni-NTA coated glass. In a fluorescence plate reader, the immobilized proteins performed similarly to their soluble counterparts (see FIGS. 28C, 28D, and 28F).

Next, a prototype device was constructed, with a light guide providing the excitation light and returning the fluorescent emitted light back to the photodetector, the bio-sensor protein immobilized to Ni-NTA coated coverslips, and the coverslip attached to the end of the light guide. The “wand” of the detector was dipped into different compositions of solutions, each with varying concentrations of maltose or maltotriose. Experiments were performed at different temperatures. PfMBP-cpGFP sensor performed better at higher temperatures (as high as 60° C.) than the EcMBP-cpGFP sensor.

### Example 3: Glutamate Indicators

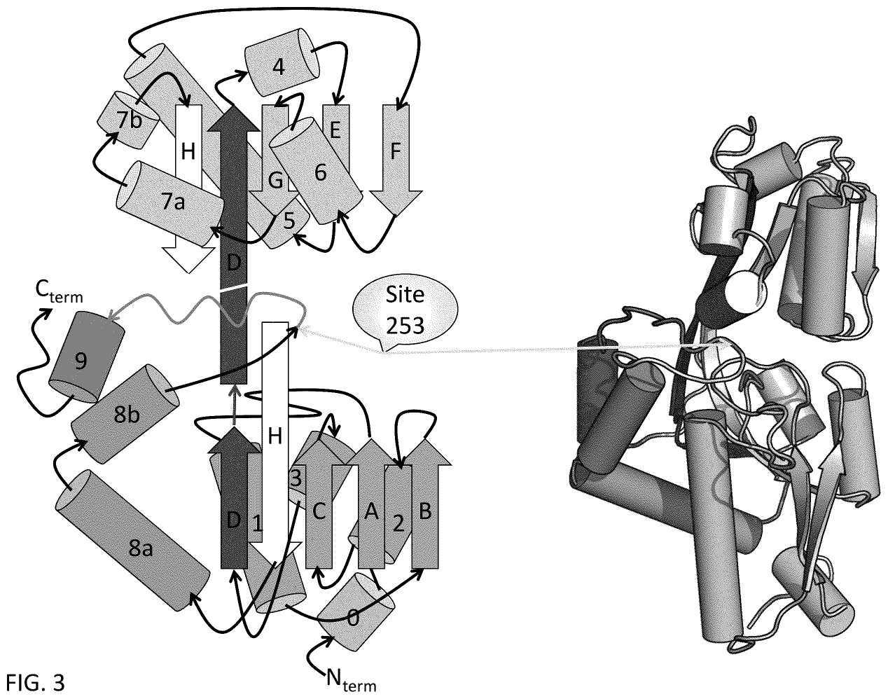

Glutamate indicators were created from Escherichia coli glutamate-binding protein (EcYbeJ). As with PfMBP in Example 2, only the structure of the ligand-bound EcYbeJ is available. EcYbeJ is homologous to EcMBP, but to a lesser degree. The best homology match between a site in EcYbeJ and a site in a binding protein for which an intensity-based sensor has already been created is EcYbeJ253 and EcMBP311 (described herein). As shown in FIG. 3, both sites are at the junction of “Rising Helix 8” and the “Equatorial Helix/Coil.” The amino acid composition of the cpGFP and EcYbeJ junction was made the same as that of the EcMBP311-cpGFP sensor (Linker 2=NP). The amino acid composition of the EcYbeJ junction and cpGFP was optimized to LV (Linker 1=LV). The variant has a ΔF/F of 5.

### Example 3A: Identification of cpGFP Insertion Sites

The ligand-bound (closed) structure of Shigella flexneri glutamate binding protein is available (Fan et al., Protein Pept. Lett., 13:513-516, 2006). This protein has only 4 amino acid mutations relative to EcYbeJ, and is thus an appropriate model.

Insertion sites for the EcYbeJ-cpGFP sensors were identified by homology to EcMBP. Based on the topology map (FIG. 3), position 311 in EcMBP was identified as an acceptable insertion site for EcYbeJ. EcMBP311 is equivalent to EcYbeJ253. EcYbeJ253 is at juncture between the end of the cluster of helices (Helices 8a, 8b, 8c) and the start of the “equatorial” spanning helix (Helix 9). In YbeJ, the structure that is homologous to the equatorial helix is the equatorial coil (depicted in red, to match the red coloring of Helix 9).

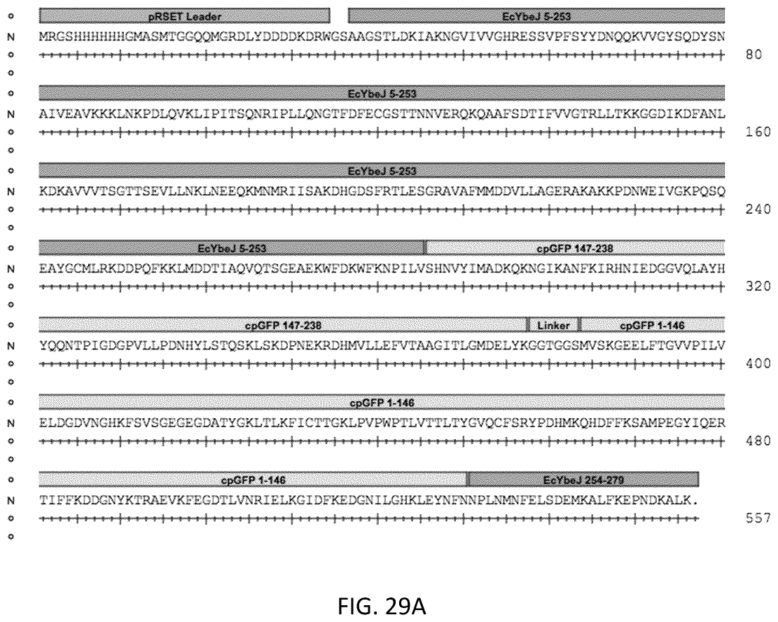

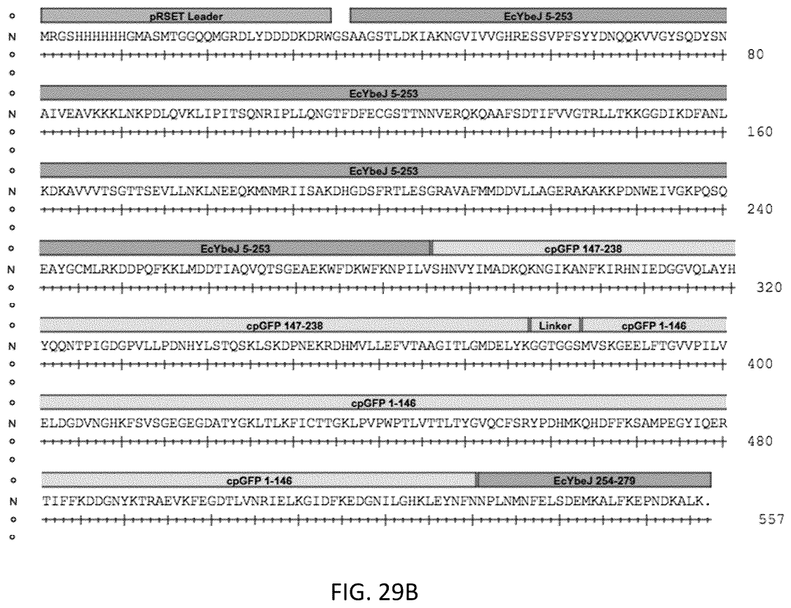

Intrinsic affinity of wild-type YbeJ for glutamate (˜1 μM) was too high to permit high-throughput screening of linker libraries. Endogenous glutamate (from the growth media) saturates the sensor, making measurement of the unbound state technically challenging. A mutation to YbeJ (A184V), in the “hinge” of the protein were made. Mutation of this residue to Trp or Arg have previously been shown to decrease affinity in FRET-based sensors (see Okumoto et al., Proc. Natl. Acad. Sci., 102:8740-8745, 2005). EcYbeJ253 (A184V)-cpGFP has an affinity for glutamate of about 100 μM. All references to EcYbeJ253-cpGFP, unless otherwise noted, refer to the A184V variant. The sequences of the EcYbeJ constructs are shown in FIG. 29.

### Example 3B: Linker Optimization

Libraries of variants of SEQ ID NOs: 62-63 were generated with randomized linkers by single-stranded uracil template mutagenesis using the primers listed below:

253 Linker 1 Primers:
(SEQ ID NO: 64)
FKNPIPPxSHNVYIMA
(SEQ ID NO: 65)
FKNPIPPxxSHNVYIMA
(SEQ ID NO: 66)
FKNPIPPPxSHNVYIMA
(SEQ ID NO: 67)
FKNPIPPxPSHNVYIMA
(SEQ ID NO: 68)
KWFKNPIxxSHNVYIMA
(SEQ ID NO: 69)
FKNPIPPxxNVYIMAD
(SEQ ID NO: 70)
KWFKNPIxxNVYIMAD
253 Linker 2 Primers:
(SEQ ID NO: 71)
KLEYNFNxKNLNMNF
(SEQ ID NO: 72)
KLEYNFNxxKNLNMNF
(SEQ ID NO: 73)
KLEYNFNxPKNLNMNF
(SEQ ID NO: 74)
KLEYNFNPxKNLNMNF
(SEQ ID NO: 75)
GHKLEYNxxLNMNF
(SEQ ID NO: 76)
KLEYNFNxxLNMNF

Where “x” indicates that a degenerate primer (with DNA sequence “NNS”) was used to encode all 20 possible amino acids.

Several thousand variants were screened in semi-high-throughput fashion, measuring fluorescence intensity of clarified cell lysate in the absence and presence of 10 mM glutamate.

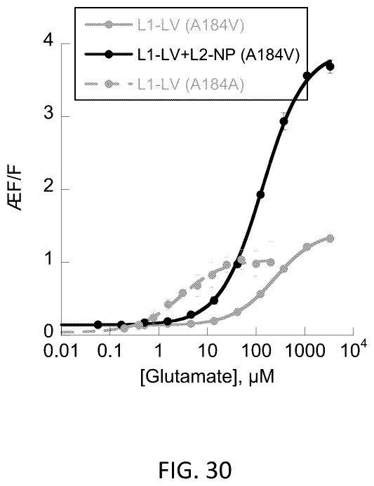

Screening a fully-degenerate, length-two library (“XX”) at the EcYbeJ253-cpGFP linker (linker 1) identified a sensor with glutamate-dependent fluorescent increases of 100%. This variant has a LeuVal EcYbeJ-cpGFP linker (L1-LV) and was used as the framework for optimization of the cpGFP-EcYbeJ253 linker (linker 2). The results of that screen yielded a protein with glutamate-dependent fluorescent increase of ˜500% and a linker 2 composition of AsnPro. As shown in FIG. 30, this variant, called “EcYbeJ253-cpGFP.L1LVL2NP” has a ΔF/F=5, a Kd for glutamate of 100 μM. Interestingly, the composition of the second linker, AsnPro, is the same as the linker composition of EcMBP311-cpGFP.L2NP.

### Example 3C: Detection of Extracellular Glutamate

EcYbeJ253-cpGFP.L1LVL2NP was cloned into the pDisplay™ vector to allow targeting and anchoring of the sensor to the plasma membrane. The resulting construct was transfected into cultured mammalian cells (HEK293) to visualize the addition of glutamate to extracellular media. Constructs were also generated in a bacterial expression vector with the epitope tags individually and in combination.

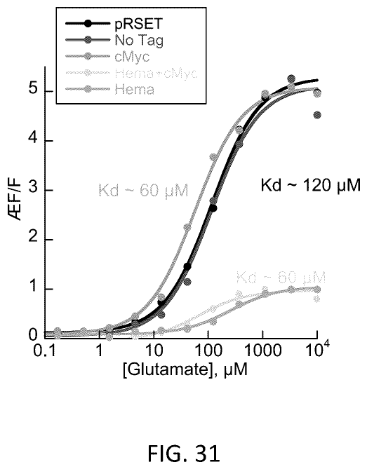

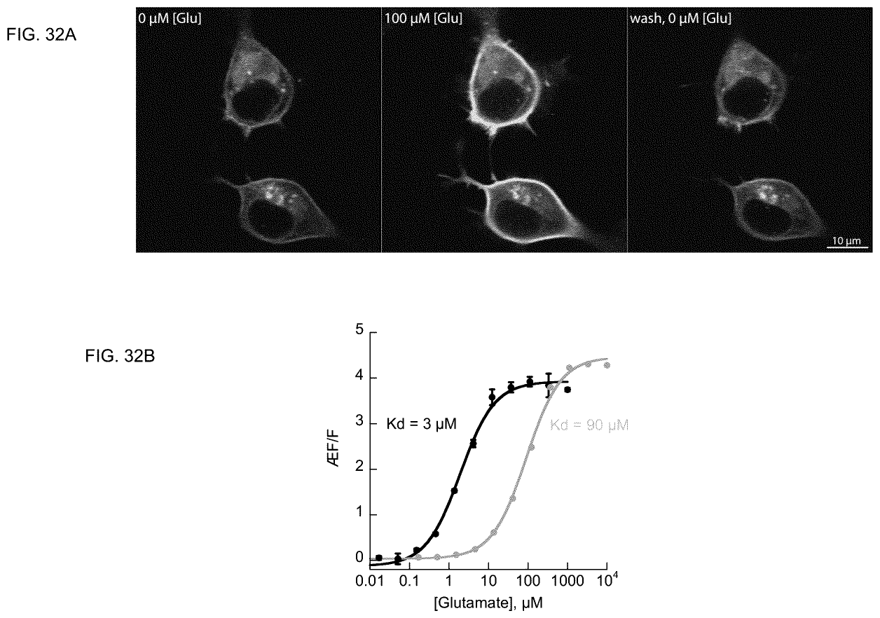

As shown in FIG. 31, the hemagglutinin tag interferes with the fluorescence change. EcYbeJ253-cpGFP.L1LVL2NP was re-cloned into a derivative of the pDisplay™ vector, lacking the hemagglutinin tag, called pMinDis (for Minimal Display). This new construct, when expressed in HEK293 cells, shows a change in fluorescence intensity under 2-photon excitation that is approximately the same as the soluble protein (see FIG. 32) with higher affinity, of about 1 (see FIG. 32).

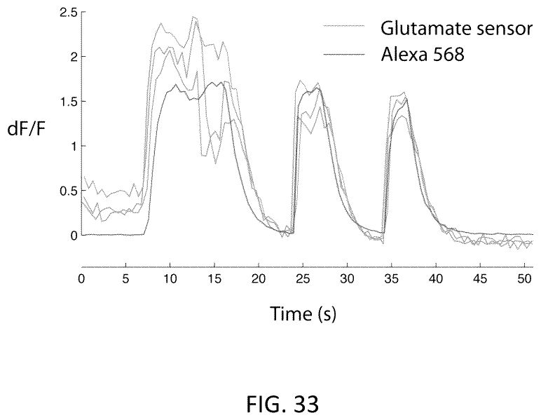

To demonstrate that the sensor is functional in neurons, and not just cultured HEK cells, the gene from EcYbeJ253-cpGFP.L1LVL2NP was cloned into an adeno-associated virus vector (AAV) under control of the synapsin promoter. Virus particles were generated and used to infect cultured primary hippocampus neurons from rats 7 days after culturing. 14 days after culturing (and 7 days after infection), the infected neurons were imaged under 2-photon microscopy (FIG. 33).

### Example 4: Phosphonate Indicators

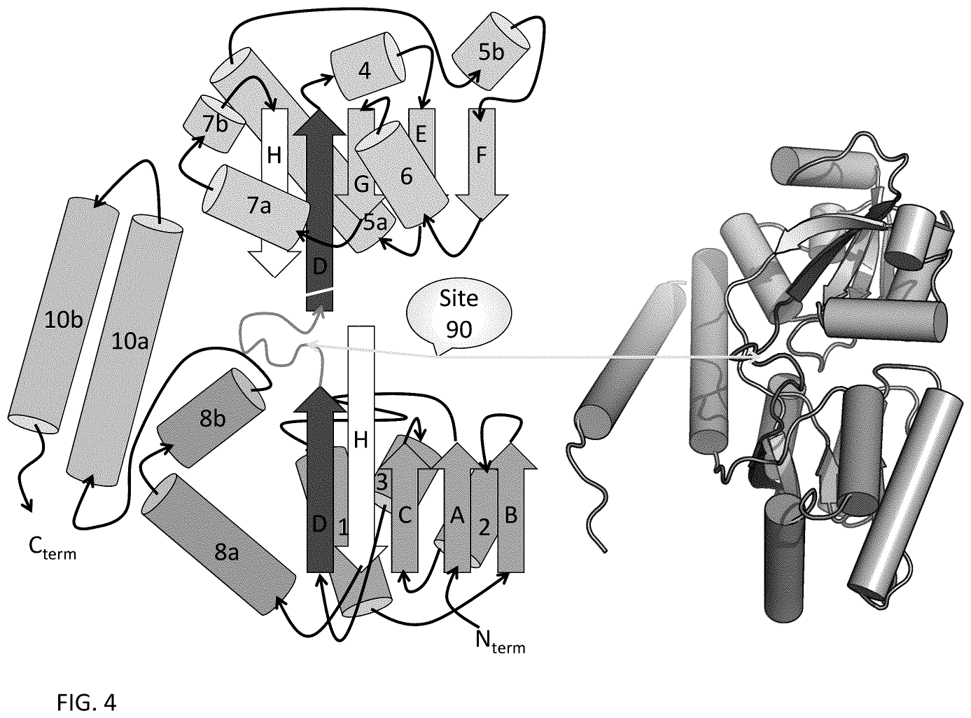

An indicator for phosphonate compounds was created from Escherichia coli phosphonate-binding protein (EcPhnD). In this instance, only the structure of the ligand-bound state was available at the time the sensor was conceived. EcPhnD is homologous to EcMBP to a lesser degree and to EcYbeJ to a greater degree. The best homology match between a site in EcPhnD and a site in a binding protein for which an intensity-based sensor has already been created is EcPhnD90 and EcYbeJ253. There is no “Rising Helix 8” in EcPhnD, but there is an “Equatorial Helix/Coil” (FIG. 4). cpGFP was inserted at the Equatorial Helix/Coil and linkers were optimized to yield a sensor with ΔF/F of 1.2. EcPhnD is a dimmer, so, a pair of mutations (L297R+L301R) were made to convert it to a monomer. The monomer variant has a ΔF/F of 1.6.

### Example 4A: Identification of cpGFP Insertion Sites in EcPhnD

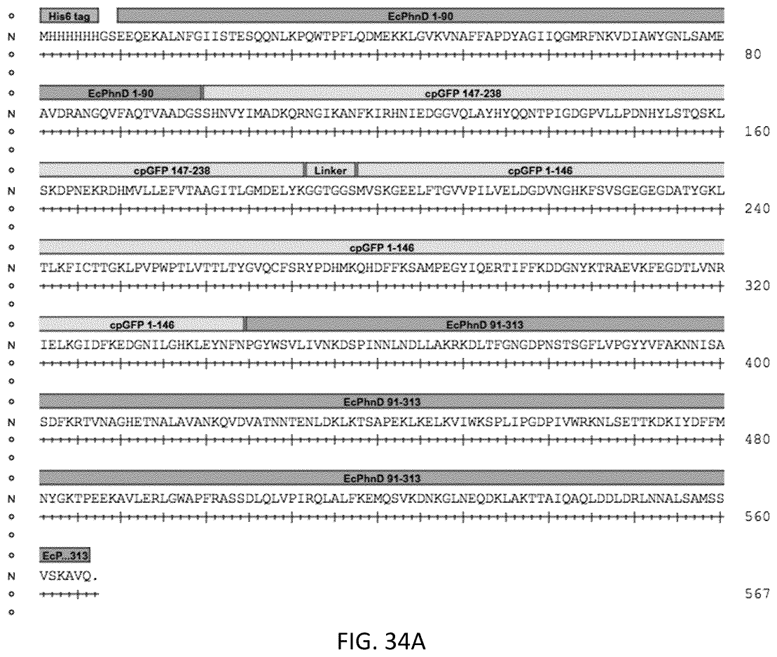

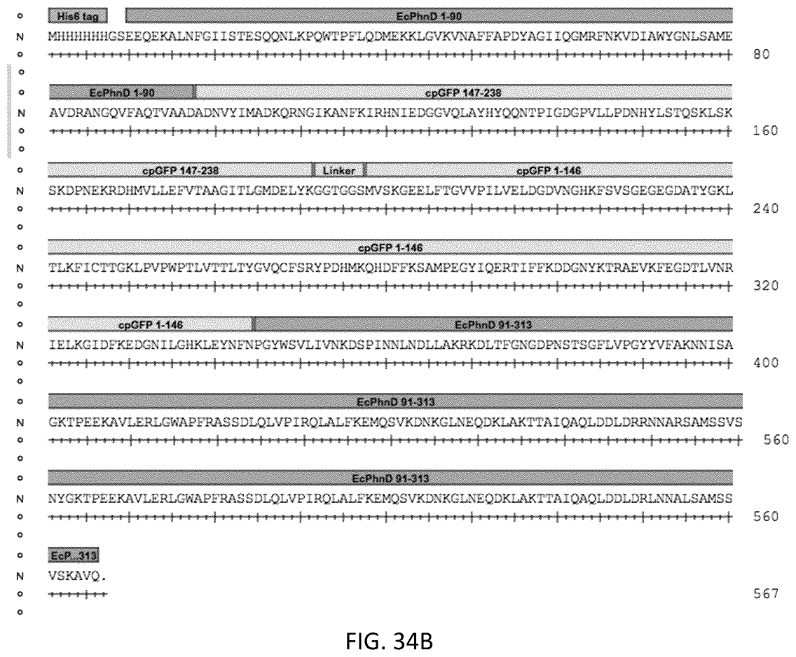

Insertion sites for the EcPhnD-cpGFP sensors were identified using the ligand-bound (closed) structure of EcPhnD by homology to EcMBP. Based on the topology map (FIG. 4), position 311 in EcMBP was identified as an acceptable insertion site in EcPhnD. EcMBP311 corresponds to EcPhnD90. This site is at the point where the rising strand (Strand D) of EcPhnD has a small bend in it that runs equatorial to the rest of the sheets in the protein. Even though it is topologically different from the “equatorial” spanning helix (Helix 9) of EcMBP its equatorial alignment is similar, and with just the closed structure at the time, in an environment that was expected to undergo significant dihedral change upon binding ligand. Sequences of EcPhnD constructs are shown in FIG. 34.

### Example 4B: Linker Optimization

Libraries of variants of SEQ ID NOs: 77-78 were generated with randomized linkers by single-stranded uracil template mutagenesis using the primers listed below:

90 Linker 1 Primers:
(SEQ ID NO: 79)
QTVAADGSSHNVYIMA
(SEQ ID NO: 80)
QTVAADxxSHNVYIMA
(SEQ ID NO: 81)
QTVAADxPSHNVYIMA
(SEQ ID NO: 82)
QTVAADPxSHNVYIMA
(SEQ ID NO: 83)
QTVAADxxNVYIMA
(SEQ ID NO: 84)
QTVAADxxSHNVYIMA
(SEQ ID NO: 85)
VFQTVAxxSHNVYIMA
90 Linker 2 Primers:
(SEQ ID NO: 86)
HKLEYNFNPGYWSVLI
(SEQ ID NO: 87)
HKLEYNFNxxPGYWSVLI
(SEQ ID NO: 88)
HKLEYNxxPGYWSVLI
(SEQ ID NO: 89)
HKLEYNFNxxYWSVLI
(SEQ ID NO: 90)
HKLEYNFNPxYWSVLI

Where “x” indicates that a degenerate primer (with DNA sequence “NNS”) was used to encode all 20 possible amino acids.

Several thousand variants were screened in semi-high-throughput fashion, measuring fluorescence intensity of clarified cell lysate in the absence and presence of 100 uM 2AEP.

Screening a number of fully-degenerate, libraries at the EcPhnD90-cpGFP linker (linker 1) yielded a protein with 2AEP-dependent fluorescent increases of >100%. This variant has a AlaAsp EcPhnD-cpGFP linker (L1-AD) and a ΔF/F of 1.2. The variant came from a linker that also deleted two residues, effectively making the insertion point of cpGFP occur after residue D88, and then skipping to residue P91 at the cpGFP-EcPhnD linker.

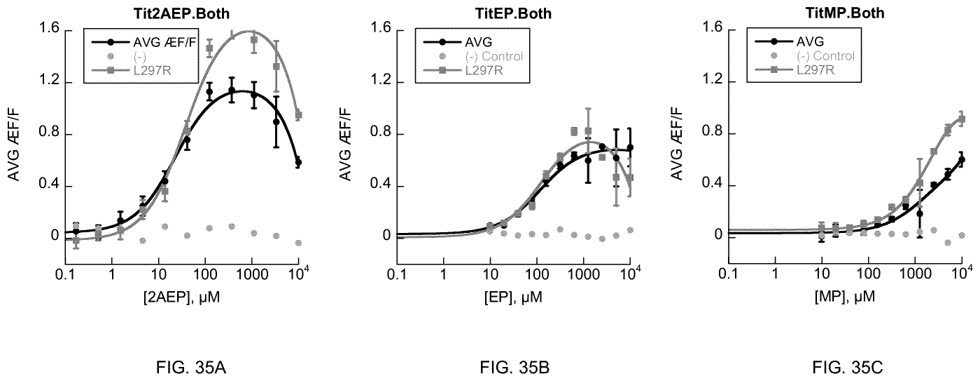

It was observed from the crystal structure that EcPhnD forms a dimer. To disrupt the dimer inter-face and potentially simplify the observable binding behavior of the EcPhnD protein, two mutations, L297R and L301R, were introduced into the dimerization helices. These mutations were expected, by charge repulsion, to disrupt the dimer interface. As shown in FIG. 35, incorporation of L279R and L301R mutations into EcPhnD90-cpGFP.L1AD caused ΔF/F to increases to 1.6 in response to 2AEP.

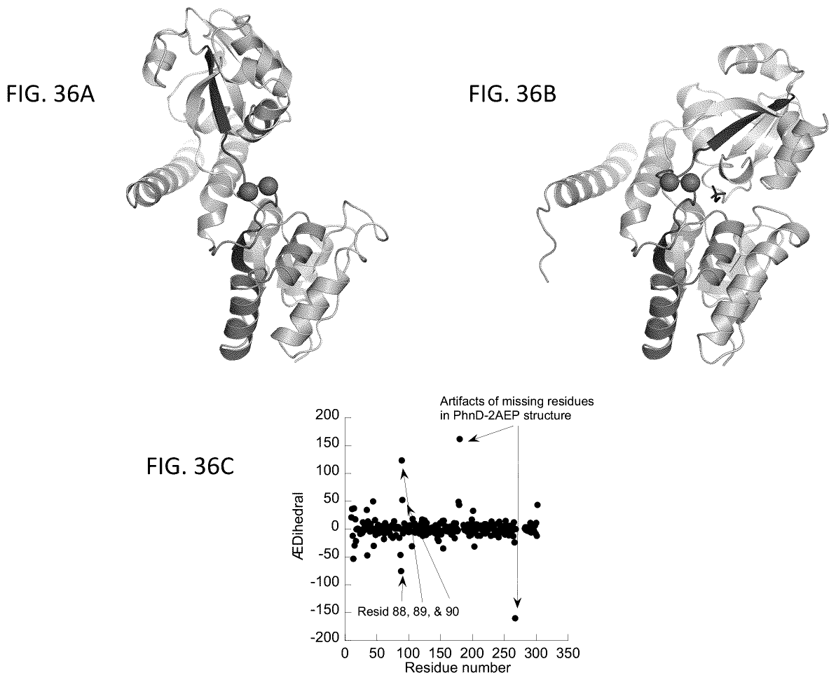

Further attempts to crystallize the open, ligand-unbound form of the protein were successful after making a mutation to the binding site, H157A, that substantially decreased affinity for phosphonate compounds. This mutant was crystallized in the absence of ligand, and the open state of the protein solved. The ΔDihedral analysis (FIG. 36) showed that the region of greatest dihedral change was the group of residues from 88-90, just one amino acid away from the site chosen by homology to the equatorial helix.

These data further indicate that ΔDihedral metric is sufficient for identifying sites in PBPs into which cpGFP can be inserted and result in intensity-based fluorescent sensors.

### Example 5: Glucose Indicators

Glucose indicators were created from Thermus thermophilus glucose binding protein (TtGBP). In this instance, only the structure of the ligand-bound state is available. TtGBP is very homologous to EcMBP and PfMBP (compare FIG. 5 with FIGS. 1 and 2). The insertion point (TtGBP326) was chosen by homology to EcMBP311 and PfMBP316. The amino acid composition of the cpGFP and TtGBP junction was made the same as that of the EcMBP311-cpGFP and EcYbeJ253 sensors (Linker 2=NP). Linker 1 was optimized (Linker 1=PA) and the TtGBP326 sensor have a ΔF/F of ˜2.5. To improve its utility for the measuring glucose concentrations in human blood, the affinity was weakened from its native ˜1 μM to 1.5 mM by mutation of two residues in the binding pocket (H66A+H348A).

### Example 5A: Identification of cpGFP Insertion Sites in TtGBP

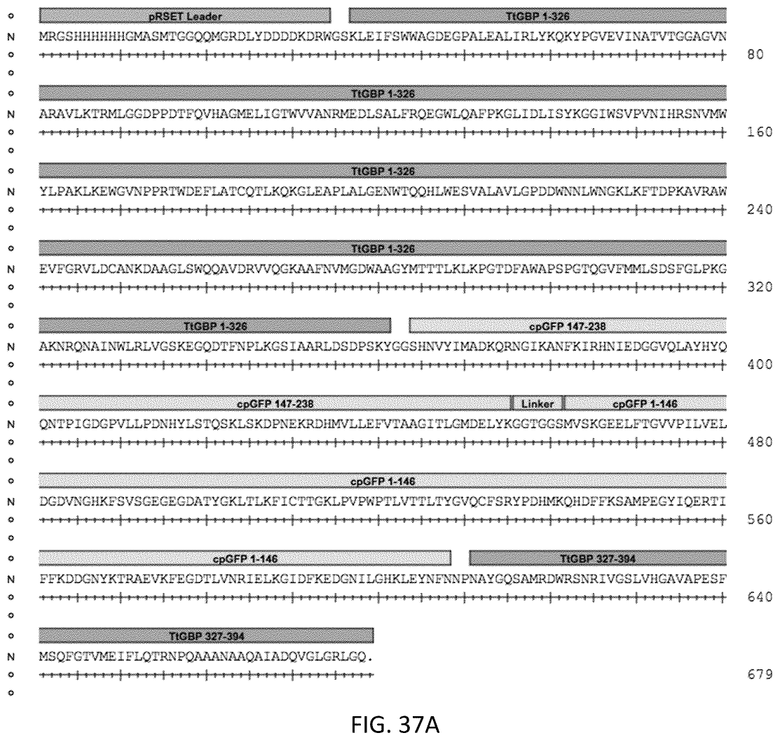

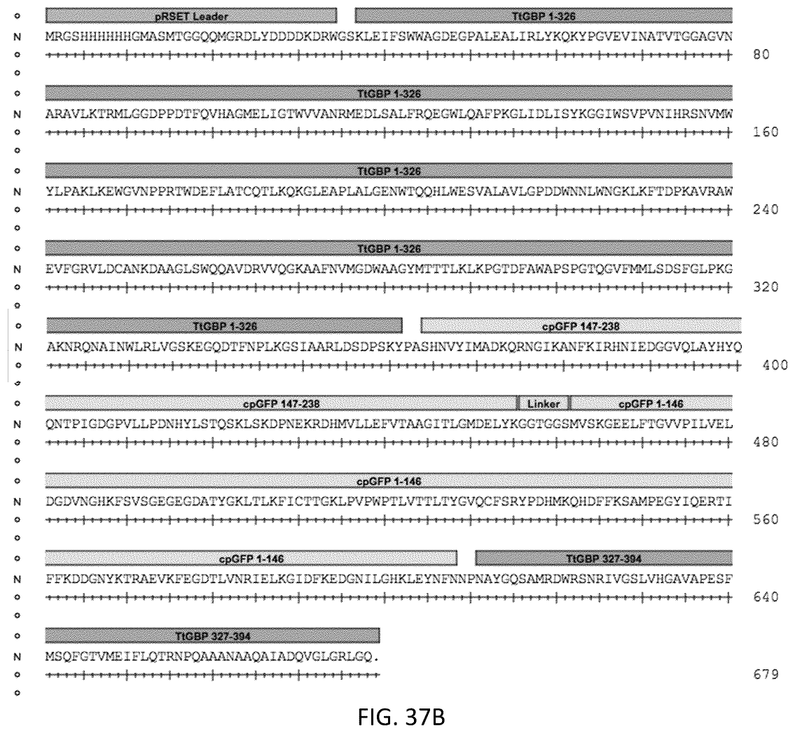

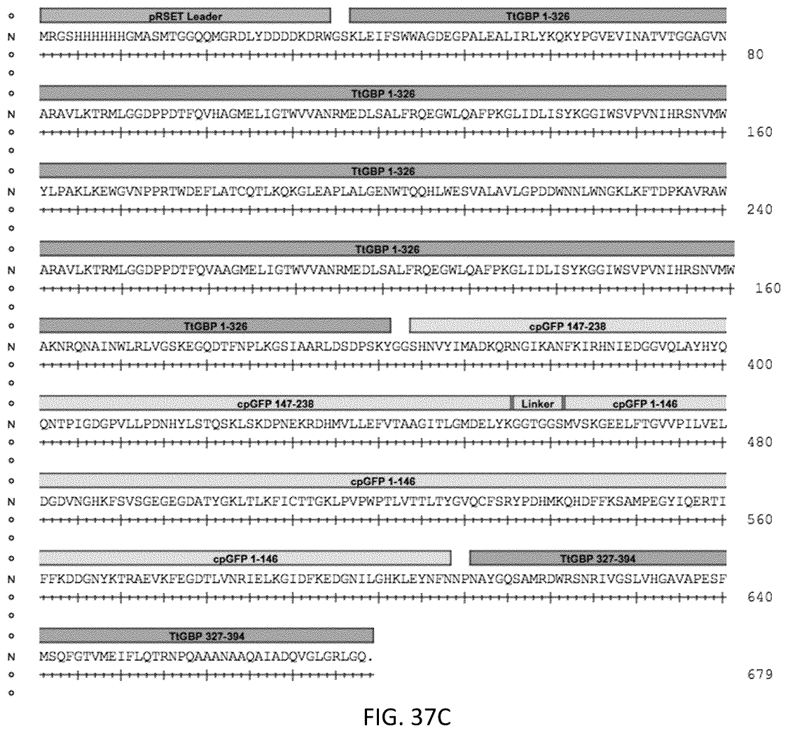

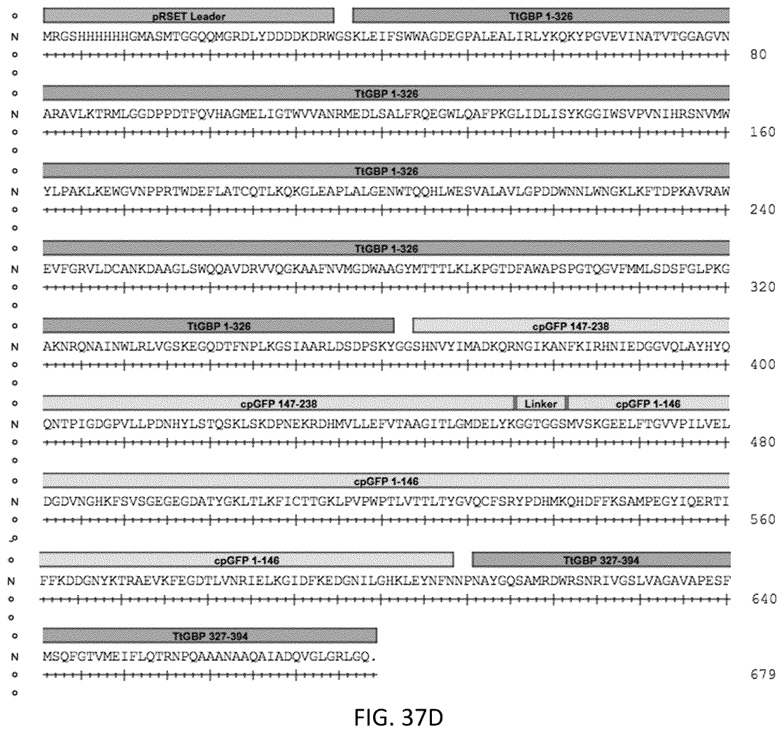

The ligand-bound (closed) structure of TtGBP is available (Cuneo et al., J. Mol. Biol., 362:259-270, 2006). Accordingly, insertion sites for the TtGBP-cpGFP sensors were identified by homology to EcMBP and PfMBP. Based on the topology map (FIG. 5), it is apparent that TtGBP, PfMBP, and EcMBP are structurally similar in the closed, ligand-bound state. Positions in EcMBP determined by the dihedral analysis (see above) were predicted to be acceptable insertion sites in TtGBP. EcMBP311 is homologous to TtGBP326. This site is at juncture between the end of the cluster of helices (Helices 8a, 8b, 8c) and the start of the “equatorial” spanning helix (Helix 9). The amino acid sequence of the TtGBP construct is shown in FIG. 37.

### Example 5B: Linker Optimization

Libraries of variants of SEQ ID NO:91 were generated with randomized linkers by single-stranded uracil template mutagenesis using the primers listed below:

326 Linker 1 Primers:
(SEQ ID NO: 95)
DSDPSKYxxSHNVYIM
(SEQ ID NO: 96)
DSDPSKYPxSHNVYIM
(SEQ ID NO: 97)
DSDPSKYxPSHNVYIM
(SEQ ID NO: 98)
RLDSDPSxxSHNVYIM
(SEQ ID NO: 99)
DSDPSKYxxNVYIM
326 Linker 2 Primers:
(SEQ ID NO: 100)
KLEYNFNxxNAYGQSA
(SEQ ID NO: 101)
KLEYNFxxPNAYGQSA
(SEQ ID NO: 102)
GHKLEYNxxNAYGQSA
(SEQ ID NO: 103)
KLEYNFNxPNAYGQSA
(SEQ ID NO: 104)
KLEYNFNPxNAYGQSA

Where “x” indicates that a degenerate primer (with DNA sequence “NNS”) was used to encode all 20 possible amino acids.

Several hundred variants were screen in semi-high-throughput fashion, measuring fluorescence intensity of clarified cell lysate in the absence and presence of 10 mM glucose.

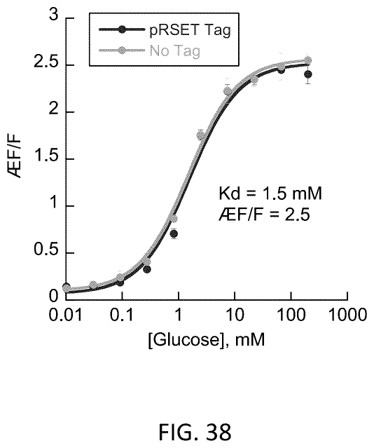

Linker 1 was optimized (Linker 1=PA) and the TtGBP326-cpGFP.L1PAL2NP sensor has a ΔF/F of ˜2.5 (see FIG. 38). Additionally, the TtGBP sensor was tested with and without the N-terminal pRSET tag and no difference was observed. Specifically, both sensors exhibited an affinity for glucose of about 1.5 mM and a ΔF/F of 2.5. Data showing that it was possible to construct a glucose sensor by replacing the EcMBP or PfMBP with TtGBP, retaining the composition of linker 2, and optimizing the composition of linker 1, indicates that the methods for generating sensors disclosed herein can be used to generate sensors using any suitable framework.

### Example 5C: Detecting Changes in Glucose Concentration in Vivo

The TtGBP326-cpGFP.L1PAL2NP sensor was cloned into a variant of the pDisplay™ vector lacking the N-terminal secretion sequence, the N-terminal hemagglutinin tag, the C-terminal cMyc tag, and the C-terminal PDGFR membrane anchoring domain.

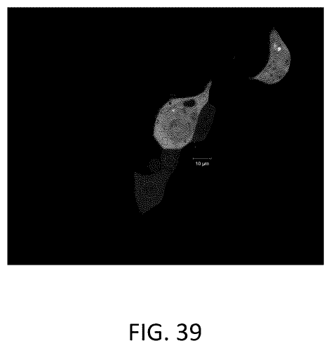

The TtGBP sensor was cloned into a mammalian expression vector (based on the pDisplay™ vector described in Example 3 above) with the secretion, epitope, and transmembrane anchoring peptides removed, thus resulting in cytosolic expression of the TtGBP326-cpGFP.L1PAL2NP+H66A+H348A sensor. The construct was transfected into HEK293 cells. As shown in FIG. 39, the TtGBP sensor was expressed in the cytosol.

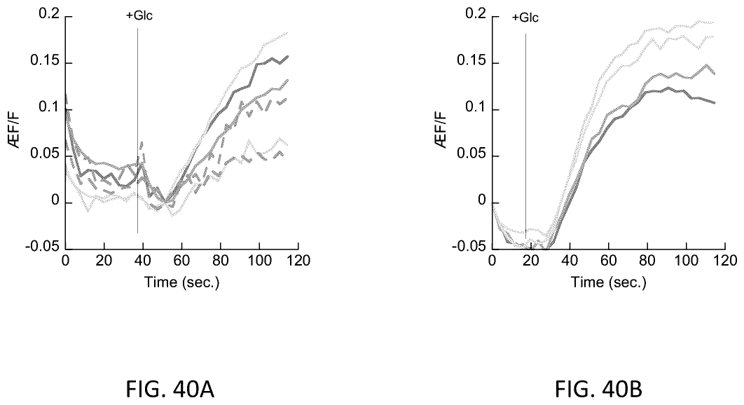

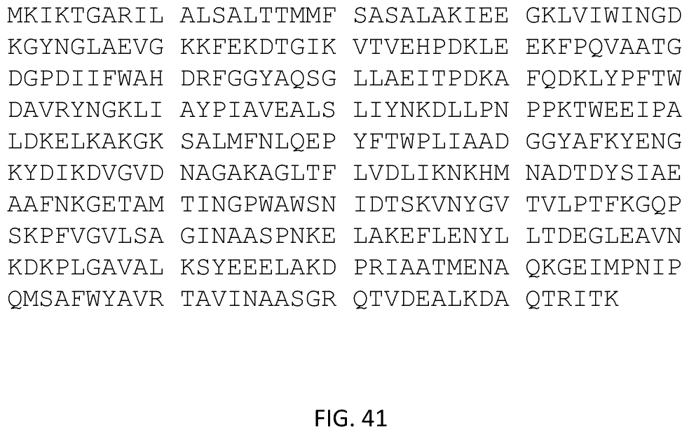

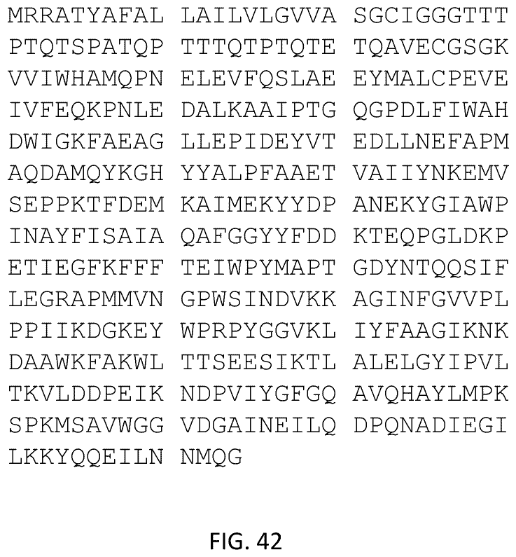

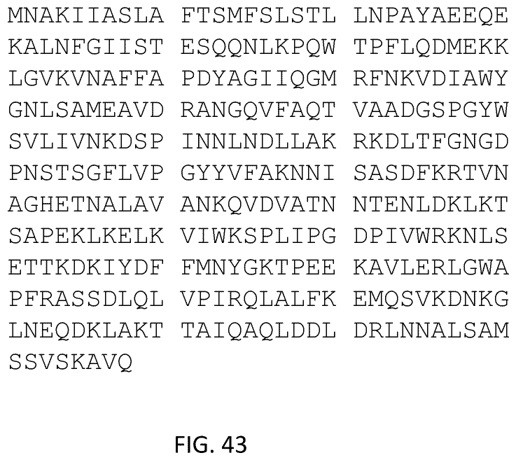

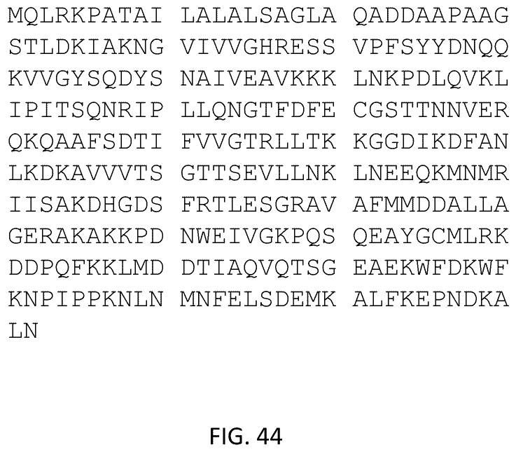

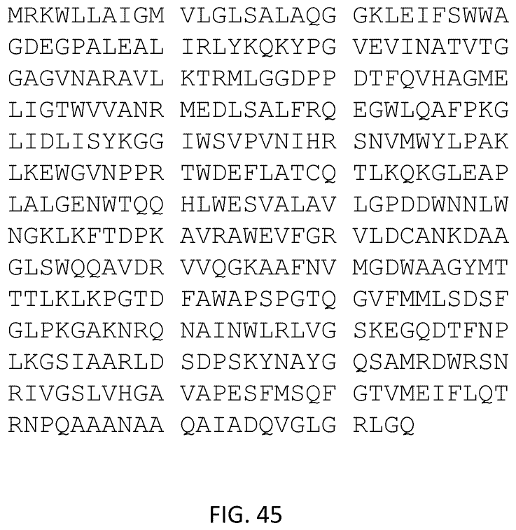

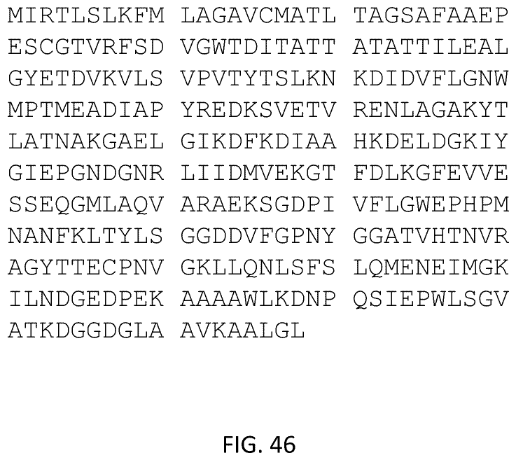

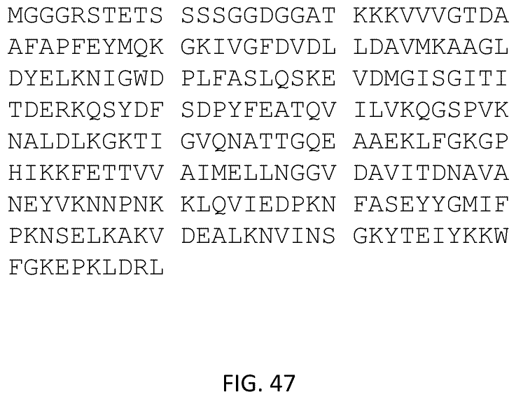

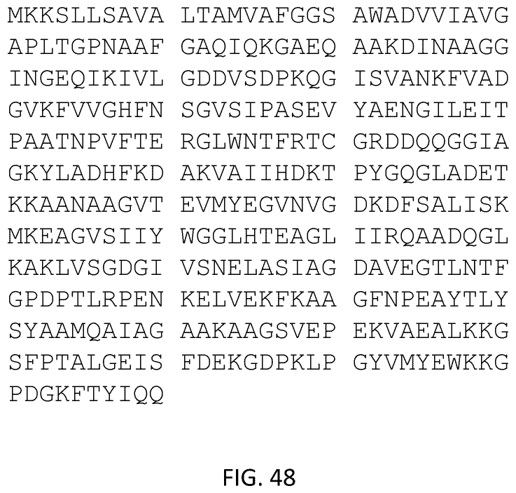

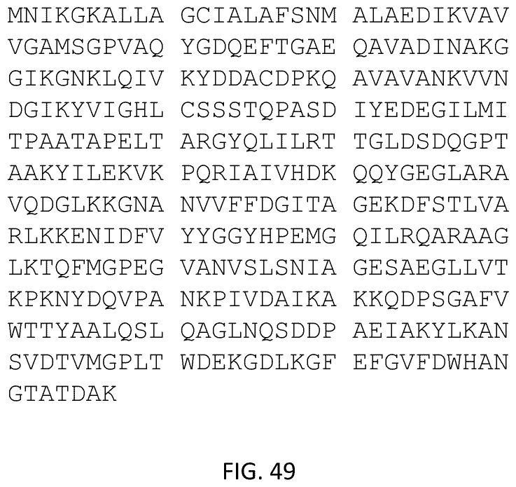

As shown in FIG. 40, addition of 10 mM glucose to the media increases fluorescence.

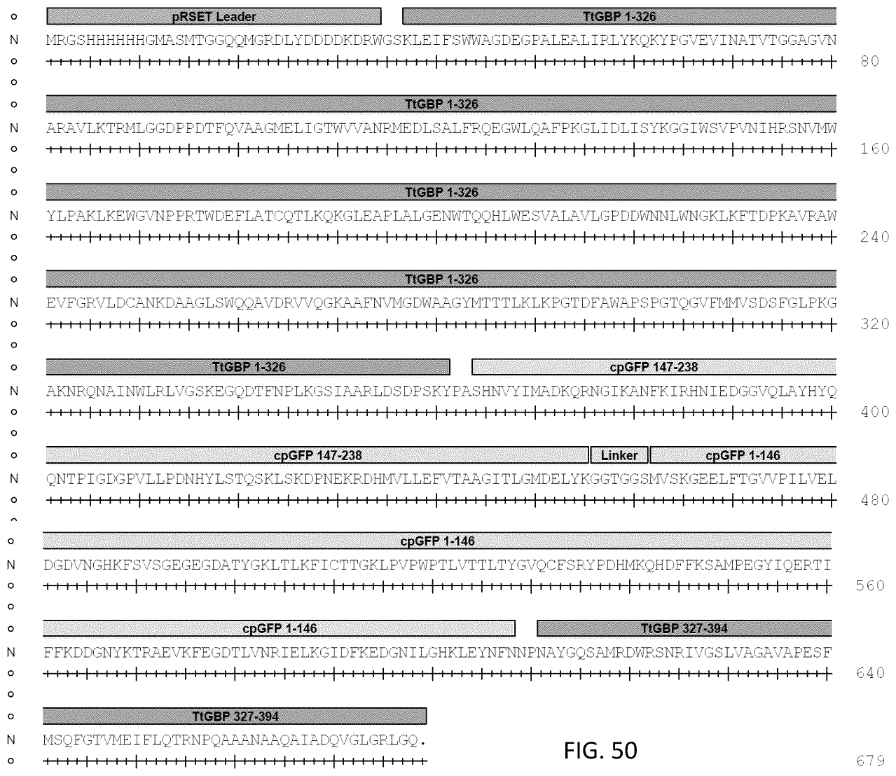

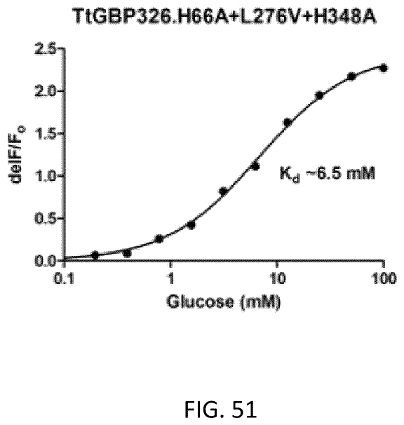

The TtGBP326-cpGFP.L1PAL2NP+H66A+H348A sensor was further modified by L276V mutation to produce TtGBP326.L1PA.L2NP.H66A.H348A.L276V (see FIG. 50). As shown in FIG. 51, this construct has an affinity for glucose of 6.5 mM.

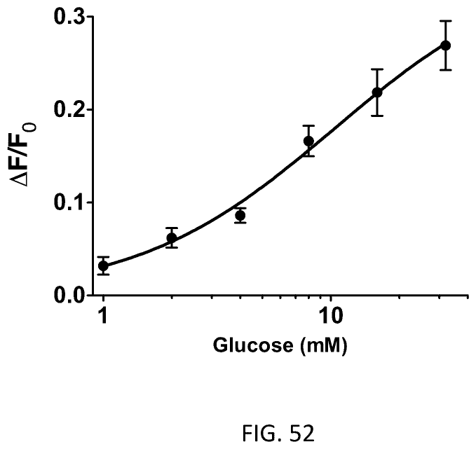

Additionally, the TtGBP326.L1P1.L2NP.G66A.H348A.L276V was cloned into the pMinDis derivative of the pDisplay vector and expressed on the extracellular surface of HEK293 cells. After exchanging the HEK293 cell media for PBS, addition of glucose to the PBS led to an increase in fluorescence (see FIG. 52).

These data indicate, in part, that the pRSET tag is not essential to the function of the sensor and that the TtGBP326-cpGFP.L1PAL2NP sensor is capable of detecting changes in the concentration of glucose inside or on the external surface of human cells.

### Example 6: Stability, Affinity and Chromatic Variants of the Glutamate Sensor iGluSnFR

### Example 6A: In Vivo Assessment of iGluSnFR Brightness in Apical Dendrites in Mouse Somatosensory Cortex

Wildtype C57/B6 mice were purchased from the Jackson Laboratory and group housed in the Janelia animal facility. Mice were injected at 8 weeks of age with AAV2/1.hSynapsin1.iGluSnFR.A184S or SF-iGluSnFR.A184S, at identical titers (1×10¹³ genomic copies per milliliter, GC/ml), volumes (20 nl), and locations (3 mm lateral to midline, 1.4 mm caudal to bregma, and 0.3 mm below the cortical surface). After viral injection, a craniotomy (3 mm diameter) was made over the injection site, and the skull was replaced with a #1.5 Schott glass and fixed in place with dental acrylic (Lang Dental Manufacturing), which also secured a titanium head bar to the skull for head-mounting during imaging experiments.

In vivo two-photon imaging experiments were performed during a state of ‘quite wakefulness’, after having been habituated to head fixation the prior 2-3 days. Period water rewards were given to keep animals hydrated and passive. For comparisons of intensity and bleaching, a custom two-photon microscope emitting 960 nm light from a Coherent Chameleon ultrafast laser was used. All experiments were performed using a 25×, 1.5 NA Olympus objective immersed in water. Image acquisition was performed with ScanImage (Vidrio) software and analyzed post hoc using ImageJ (NIH). Images were acquired at a variety of speeds/zooms, and powers in order to assess the impact of pulse energy and dwell time on bleaching and intensity. Images at each setting were acquired for 5 seconds. To analyze the data, images were averaged and thresholded to create a signal (above threshold) and background mask. Signals in these masks were then averaged, and SNR was calculated from these as (signal-background)/(standard deviation of background). Bleaching percentage was calculated as the average intensity in the first 25% of the trace, divided by the last 25% of the trace.

### Example 6B: Ferret Visual Cortex Assessment of SF-iGluSnFR.A184S and A184V

All procedures were approved by the Max Planck Florida Institute for Neuroscience Institutional Animal Care and Use Committee and adhered to the standards of the National Institutes of Health. Juvenile female ferrets (Mustela putorius furo, Marshall Farms) were used. Animals were housed in a vivarium under 16 hour light/8 hour dark cycle. The full methodological details for functional two-photon imaging of ferret visual cortex is previous described in detail (Wilson et al., 2016, Nat. Neurosci., 19:1003-9).

Briefly, juvenile female ferrets (Mustela putorius furo, Marshall Farms) aged P21-22 (n=2) were anesthetized with ketamine (50 mg/kg, IM) and isoflurane (1-3%) delivered in O₂, then intubated and artificially respirated. Atropine (0.2 mg/kg, SC) and a 1:1 mixture of lidocaine and bupivacaine administered subcutaneously in the scalp. Animals were kept at 37° C. A small craniotomy (0.8 mm) was made over the visual cortex 7-8 mm lateral and 2-3 mm anterior to lambda. AAV2/1.hSynapsin1.Cre (Penn Vector Core) was diluted in phosphate-buffered saline (Sigma) and mixed with AAV2/1.hSynapsin-FLEX.SF-iGluSnFR.A184S or A184V for expression in layer 2/3 cortical neurons. Beveled glass micropipettes were lowered into the brain and 400-500 nl of virus were injected over 5 minutes at multiple depths below the pia. Following, the craniotomy was filled with 1% w/v agarose.

After four weeks, ferrets were anesthetized with 50 mg/kg ketamine and 1-3% isoflurane. Atropine (0.2 mg/kg, SQ) and bupivacaine were administered. Animals were kept at 37 to 38° C., artificially respirated, and given intravenous fluids. Isoflurane (1-2%) was used throughout the surgical procedure to maintain a surgical plane of anesthesia. ECG, endtidal CO₂, external temperature, and internal temperature were continuously monitored. A custom titanium headplate was implanted on the skull at the viral injection site and the dura retracted to reveal the cortex. A custom insert with a single 4 mm coverglass (0.17 mm thickness) was placed onto the brain to gently compress the underlying cortex and dampen biological motion during imaging. The cranial window was hermetically sealed using a stainless steel retaining ring and Vetbond. Tropicamide Ophthalmic Solution and Phenylephrine Hydrochloride Ophthalmic Solution were applied and contact lenses were inserted into both eyes. Upon completion of the surgical procedure, Isoflurane was gradually reduced and pancuronium (2 mg/kg/hour) was delivered IV to immobilize the animal.

The animal was placed under the microscope 25 cm from the stimulus monitor, with the monitor subtending 130 degrees in azimuth and 74 degrees in elevation. Imaging was performed using a Bergamo II (Thorlabs) running ScanImage 5 or ScanImage 2015¹⁹ (Vidrio Technologies) with dispersion compensated 950 nm excitation provided by an Insight DS+ (Spectraphysics). Average excitation power after the exit pupil of the objective (16×, CFI75, Nikon Instruments) ranged from 25 to 40 mW. Two-photon frame triggers from ScanImage were synchronized with stimulus information using Spike2 (CED). Visual stimuli were generated using PsychoPy (Peirce, 2007, J. Neurosci. Methods, 162:8-13). Full-field drifting square-wave gratings (16 directions, 100% contrast, 0.1 cycles/°, 4 cycles/sec., 3 sec. stimulus period followed by 2-3 sec. ISI, plus a blank) were presented to the contralateral eye in a pseudorandom sequence for 8 trials.

Images were corrected for in-plane motion using a correlation-based approach (MATLAB). ROI drawing was performed in ImageJ (Schindelin et al., 2012, Nat. Methods, 9:676-82). Fluorescence time-courses were computed as the mean of all pixels within the ROI at each time point and were extracted as described in Sage et al. (2012, ImageJ User developer Conference 1:1). Fluorescence time courses were then synchronized with stimulus information, and visually evoked responses were computed as changes in fluorescence relative to the baseline fluorescence. Peak ΔF/F responses for field ROIs and dendritic spines ROIs were computed using the Fourier analysis to calculate mean and modulation amplitudes for each stimulus presentation, which were summed together.

### Example 6C: Mouse Neuronal Culture Analysis

Primary Hippocampal Neuron Cultures

Primary hippocampal neuron cultures were prepared from embryonic mice (E16) as described previously (Woitecki et al., 2016, J. Neurosci., 36:2561-70). Hippocampi were rinsed 3-5 times in Hank's Balanced Salt Solution (HBSS, Life technologies) and digested with trypsin (25 mg/ml, Life Technologies) for 20 min at 37° C. followed by DNase I (1 mg/ml; Roche). Subsequently, the tissue was dissociated using cannulas (three times 0.9 mm×40 mm; three times 0.45 mm×23 mm) and the solution was passed through a Nylon cell strainer (100 μm; BD Biosciences). The mesh was rinsed with 4-10 ml basal medium eagle (BME, Life technologies) supplemented with 0.5% glucose (Sigma-Aldrich), 10% fetal calf serum (FCS), 2% B-27, and 0.5 mM L-glutamine (all Life Technologies) to collect all cells. After counting, the cells were plated on cover slips in a 24-well cell culture plate at a density of 70,000 cells per 24-well and cultured in a humidified incubator at 37° C. and 5% CO₂.

Viral Vector Production

Recombinant AAV2/1 genomes were generated by large scale triple transfection of HEK293 cells as described previously (Marvin et al., 2013, Nat. Methods, 10:162-70). The adeno-associated virus (AAV) plasmid coding for SF-iGluSnFR.S72A or SF-iGluSnFR.A184V, helper plasmids encoding rep and cap genes (pRV1 and pH21), and adenoviral helper pFA6 (Stratagene) were transfected using the calcium phosphate transfection method. Cells were harvested ˜72 h after transfection. To purify the virus, cell pellets were lysed in the presence of 0.5% sodium deoxycholate (Sigma) and 50 units/ml Benzonase endonuclease (Sigma). rAAV viral particles were purified from the cell lysate by HiTrap heparin HP column purification (GE Healthcare) and then concentrated using Amicon Ultra Centrifugal Filters (Millipore) until a final stock volume of 500 μl was reached.

Viral Transduction and Image Acquisition

Primary hippocampal neurons were transduced with AAV2/1.hSynapsin1.SF-iGluSnFR.S72A or with AAV2/1.hSynapsin1.SF-iGluSnFR.A184V on DIV4 and imaged on DIV13. A low amplitude field stimulation (1 msec, 20 mA, platinum bar electrodes) was applied to recruit a small fraction (˜20%) of neurons. Images were acquired with an EM-CCD camera (frame time 5-50 msec) and a stabilized LED light source of cultures visualized through a coverslip with high NA objective. All experiments were performed in Tyrode's solution (1 ml/min) at RT. Low and high affinity versions of SF-iGluSnFR were expressed in a comparable manner.

Glutamate Release Site Localization

Primary hippocampal neurons were transduced with rAAV-SF-iGluSnFR. S72A or with rAAV-SF-iGluSnFR.A184V on DIV3-5 and used for experiments on DIV13-18. A low amplitude electrical field stimulation (1 msec., 20 mA, platinum bar electrodes) was applied to activate a small fraction (˜20%) of neurons only. Per experiment, stimuli were applied 16-times at an inter-stimulus interval of 20-60 sec. Images were acquired with an EM-CCD camera (Hamamatsu ImagEM X1, 8 ms exposure, 125 Hz acquisition rate) attached to an inverted microscope (Nikon T1 Eclipse) using a triggered, stabilized LED light source (Cairn OptoLED with 470 nm excitation wavelength, 470/40 emission filter and 525/50 excitation filter). Cells were imaged through a coverslip with a high NA objective (Zeiss, 63×, 1.4 NA, water). All experiments were performed in saline (1 ml/min, as described above) at room temperature.

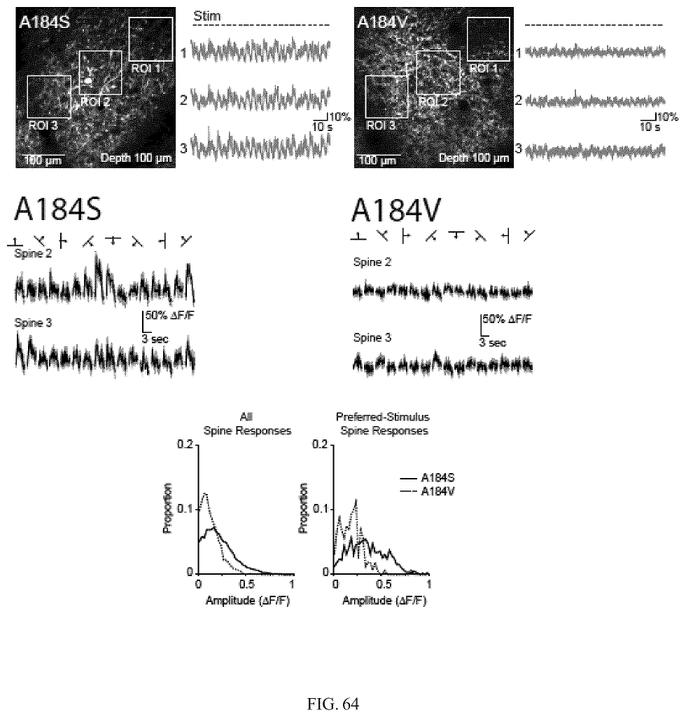

In each experiment, 30 images were acquired per stimulation trial (20 before and 10 after stimulation). Each of the 30 images was registered with StackReg Plugin in ImageJ to the first image. The image series was then normalized to the average of 5 frames before stimulation to distinguish responding sites (>1) and non-responding structures (˜1). For selection of responding sites to be included in the analysis, 10 normalized images subsequent to the stimulus in the first trial were averaged. All spots of increased fluorescence (FIG. 64c,d) that reached at least 50% of the ΔF/F value of the brightest spot in the image were defined as responding sites and used for further analysis. The spatial extent of glutamate release sites was quantified by extracting a brightness profile based on a line (length: 12-30 pixels, width: 3 pixels) drawn along the underlying neurite. These profiles were calculated for each stimulation trial and each responding site in an experiment and fitted by Gaussians with Igor Pro 6.3 (Wavemetrics).

In each experiment (n=6 and 8 for S72A and A184V, respectively, each consisting of 16-25 trials) the mean deviation of the center (X₀ position), the average width and the average amplitude of the fitted Gaussians were calculated per response site and averaged across all experiments and statistically compared by an unpaired Mann-Whitney test, n=28 and 53 for S72A and A184V, respectively).

### Example 6D: Cerebellar Parallel Fiber Analysis

Stereotaxic Injections.

To fluorescently label boutons of parallel fibers, stereotaxic injections of viral vectors expressing SF-iGluSnFR or GCaMP6f into cerebellar vermis were performed. The following vectors were used: AAV-DJ.hSynapsin.SF-iGluSnFR (1.9×10¹³ GC/ml), AAV2/1.hSynapsin.SF-iGluSnFR.S72A (2.6×10¹³ GC/ml), AAV-DJ.CAGFLEX.SF-iGluSnFR.S72A (6.3×10¹² GC/ml) or AAV-DJ.hSynapsin.GCaMP6f (1.2×10¹³ GC/ml). Mice between 30 and 60 days old were deeply anesthetized before surgery with a mixture of hypnotic (ketamine 1.5%, Mérial) and analgesic (xylazine 0.05%, Bayer) anesthetics mixed in NaCl and injected in the peritoneum. A local anesthetic (xylocaine 2% gel, Newpharma) was applied on top of the location of the cranial incision. The anesthetized mouse was then placed on a stereotaxic frame adaptor comprising adjustable ear bars and tooth holder. The skull was then perforated at the injection site with a surgical drill. The vermis was identified using the Paxinos and Franklin mouse brain atlas. The injection of viral constructs in the vermis (100 nl; 6.5 mm caudal to bregma, lateral 0.2 mm, ventral 3.6 mm and 3.4 mm) was performed by slow infusion (100 nl/min) with steel needles (26 G×50 mm and 36 G×70 mm, Phymep) connected to a pump via a catheter and a Hamilton syringe. Injected mice were then kept 2 to 4 weeks to allow transgene expression.

Slice Preparation

All protocols were approved by the ethics committee CEEA-Paris1. Cerebellar acute slices were prepared from adult CB6F1 mice (F1 cross of BalbC and C57Bl/6J) or Gabra6 mice (B6; 129P2-Gabra6^{tm2(cre)Wwis}/Mmucd) of postnatal day 41 to 123. The mice were killed by rapid decapitation, after which the brains were quickly removed and placed in an ice-cold solution containing (in mM): 2.5 KCl, 0.5 CaCl₂, 4 MgCl₂, 1.25 NaH₂PO₄, 24 NaHCO₃, 25 glucose, 230 sucrose, and 0.5 ascorbic acid bubbled with 95% 02 and 5% CO₂. Coronal slices were cut from the dissected cerebellar vermis using a vibratome (Leica VT1200S). After preparation, the slices were incubated at 32° C. for 30 minutes in the following solution (in mM): 85 NaCl, 2.5 KCl, 0.5 CaCl₂, 4 MgCl₂, 1.25 NaH₂PO₄, 24 NaHCO₃, 25 glucose, 75 sucrose and 0.5 ascorbic acid. Slices were then transferred to an external recording solution containing (in mM): 125 NaCl, 2.5 KCl, 1.5 CaCl₂, 1.5 MgCl₂, 1.25 NaH₂PO₄, 25 NaHCO₃, glucose and 0.5 ascorbic acid, and maintained at room temperature for up to 6 hours. All slice recordings were performed at 36-38° C.

Transmitted Light and Fluorescence Imaging

Parallel fiber and boutons expressing SF-iGluSnFR or GCaMP6f were identified using an Ultima two-photon scanning scanhead (Bruker Nano Surfaces Division, Middleton, WI, USA) that was mounted on an Olympus BX61W1 microscope, equipped with a water-immersion objective (60×, 1.1 NA, Olympus Optical, Tokyo, Japan) and infrared Dodt-gradient contrast. Two-photon excitation was performed with a pulsed Ti:Sapphire laser (DeepSee, Spectra-Physics, France) tuned to 920 nm for imaging morphology, glutamate and Ca²⁺ fluorescence detection.

Boutons from parallel fibers were identified by increase fluorescence as response to 100 or 300 Hz trains. The probe response was evoked with 60 μs voltage pulses 5-15 V above threshold (Digitimer Ltd, Letchworth Garden City, UK) using a patch pipette (typically with a tip resistance of 4-6 MΩ) filled with ACSF and placed in the molecular layer adjacent to labelled parallel fibers. Activation of boutons was routinely confirmed by verifying increase in fluorescence in response to 100 or 300 Hz trains of stimulation. Line-scan imaging through boutons was performed at dwell time of 0.8 μsec per pixel, for 300 to 800 msec. Individual traces were background subtracted and averaged with no smoothing or filtration for single events for SF-iGluSnFR, or background subtracted and averaged with smoothing for GCaMP6f, 20 Hz and 100 Hz trains. SNR was calculated from the peak of the fit to the fluorescent events divided by the average SD of a 20 msec baseline window. Data were analyzed and presented using custom-written macros in Igor Pro.

### Example 6E: Fast Imaging of SF-Venus-iGluSnFR

Primary Rat Hippocampal Neuron Cultures

A mixed cell culture (neurons and glia) was prepared from Sprague-Dawley rat pups (Charles River Laboratories). Briefly, P0 pups were decapitated, and the brains were dissected into ice-cold neural dissection solution (NDS, 10 mM HEPES (Sigma) in HBSS (Invitrogen), pH 7.4). Hippocampi were dissected and cut into small pieces to facilitate enzyme digestion. Hippocampi pieces were transferred using a large bore pipette into a 15 ml conical tube and incubated with enzyme digest solution (Papain, Worthington Biologicals) at 37° C. for 30 min. After 30 min., the enzyme solution was removed, and Plating Media (MEM media containing 10% FBS) was added and tissue pieces were triturated resulting in mostly single cells. The cell suspension was filtered using a 45 um filter. The filtered cell suspension was centrifuged, and the resulting cell pellet was re-suspended with Plating Media and counted.

For electroporation, 1 μg of DNA was mixed with 1×10⁶ cells using the Amaxa Nucleofector II instrument. Cells were plated onto coverslips coated with Poly-D-Lysine (Sigma) and kept at 37° C., 5% CO₂ in PM for ˜24 hours and then in NbActiv4 (BrainBits) was added for the duration with medium exchanges every 4 days.

Glutamate Uncaging and Imaging

Rat hippocampal culture was imaged on DIV19 at room temperature in HEPES buffered Tyrode's solution (145 mM NaCl, 2.5 mM KCl, 10 mM glucose, 10 mM HEPES, 2 mM CaCl₂, 1 mM MgCl₂, pH 7.4).

Excitation was with a 1030 nm, 5 MHz, 190 fsec laser (Menlo Systems, model: Bluecut). Average power was 39 mW at the sample. Fluorescence collected at 560/80 nm with a Hamamatsu MPPC detector. The field of view is a 256 um diameter circle, 1280 pixels across. The bath contained HEPES buffered Tyrode's solution plus 10 μM NBQX and 150 μM RuBi-Glutamate (Tocris). Glutamate uncaging was performed with 420 nm fiber-coupled LEDs (Thorlabs M420F2). The tips of the fibers were imaged onto the sample plane through the same objective used for activity imaging.

### Example 6F: Summary of Results

The intensity-based glutamate-sensing fluorescent reporter (iGluSnFR) (Marvin et al., 2013, Nat. Methods, 10:162-70) has become an invaluable tool for studying glutamate dynamics in diverse systems, including retina (Park et al., 2014, J. Neurosci., 34:3976-81; Borghuis et al., 2013, J. Neurosci., 33:10972-85), mouse olfactory bulb (Brunert et al., 2016, J. Neurosci., 36:6820-35) and cat visual cortex (O'Herron et al., 2016, Nature, 534:378-82). Beyond specific circuits, iGluSnFR also allows mesoscale “functional connectomic” mapping (Xie et al., 2016, J. Neurosci., 36:1261-72) and mechanistic studies of Huntington's disease (Jiang et al., 2016, J. Neurosci., 36:3453-70), synaptic spillover (Rosa et al., 2015, eLife, 4:728), cortical spreading depression (Enger et al., 2015, Cerebral Cortex, 25:4469-76) and exocytotic vesicle fusion (Bao et al., 2016, Nat. Struct. Biol., 23:67-73). However, iGluSnFR is insufficient for some applications due to poor expression (in some brain regions), and kinetics that do not match the time courses of some observations. Here, we describe variants that are functionally brighter (due to increased expression on cell membrane), have tighter or weaker affinity (resulting from slower or faster off-rates), and fluoresce blue, green, or yellow.

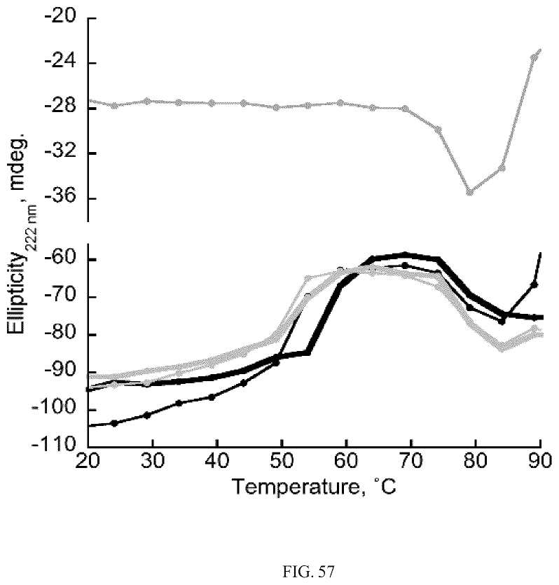

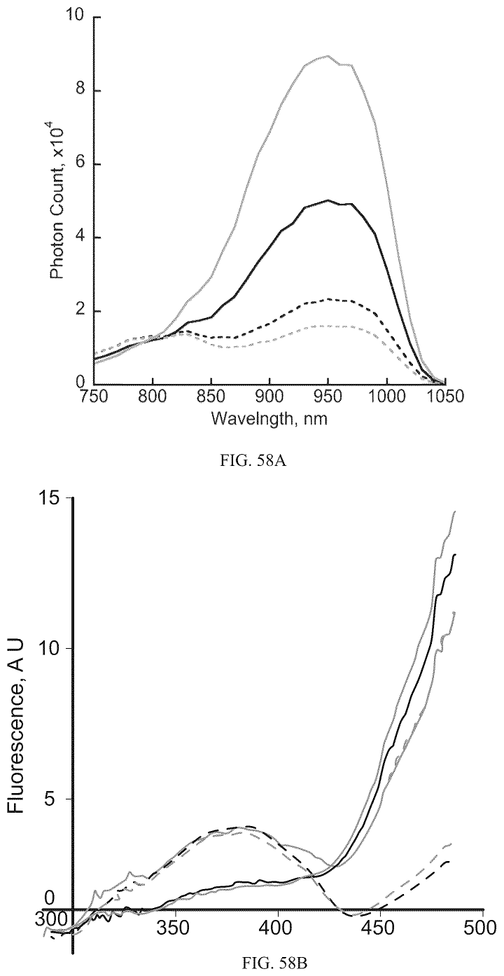

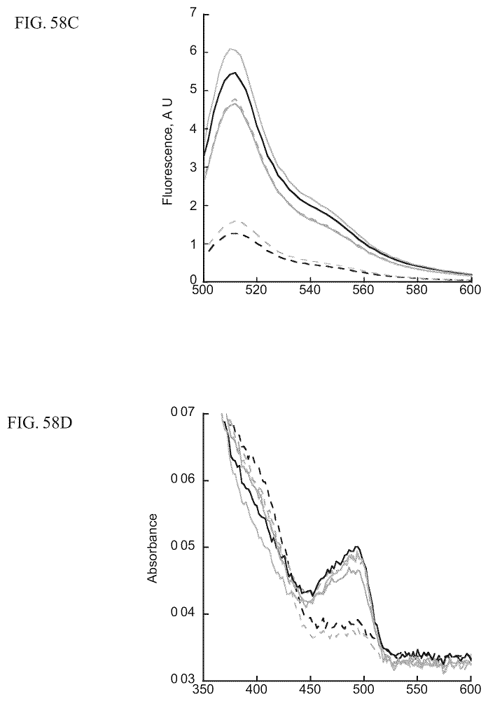

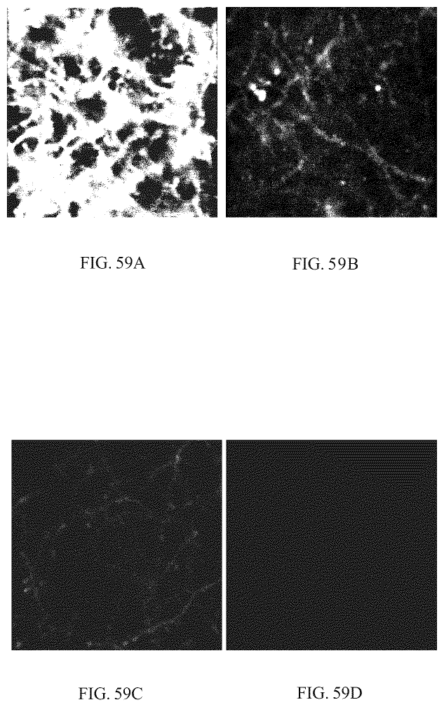

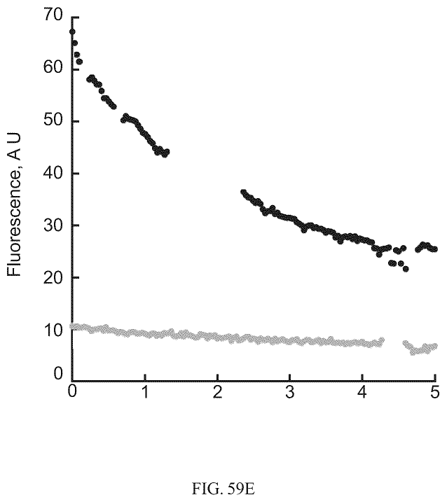

Replacement of circularly permuted eGFP with circularly permuted “superfolder” GFP (Pedelacq et al., 2006, Nat. Biotech., 24:79-88) (SF-iGluSnFR) yielded 5-fold higher soluble-protein expression levels in bacteria (0.5 μmol/1 L growth vs. 0.1 μmol/1 L). Circular dichroism indicates an increase in melting temperature transition (Tₘ) of ˜5° C. (FIG. 57). The 2-photon cross-section and excitation, emission, and absorption spectra of SF-iGluSnFR are similar to the original (FIG. 58a-d). Head-to-head comparison of SF-iGluSnFR with original iGluSnFR in mouse somatosensory cortex shows substantially more robust expression by the former (FIG. 59a,b). Under typical imaging conditions (<20 mW, 130-nanosecond dwell time per pixel), SF-iGluSnFR is bright enough for repeated imaging, while original iGluSnFR is too dim (FIG. 59c,d). While a faster 2-photon in vivo photobleaching rate was observed for SF-iGluSnFR in somatosensory cortex (FIG. 59e), partially-bleached SF-iGluSnFR was still brighter than iGluSnFR. Thus, SF-iGluSnFR will have superior expression in vivo, where the quantity of deliverable DNA can be limiting.

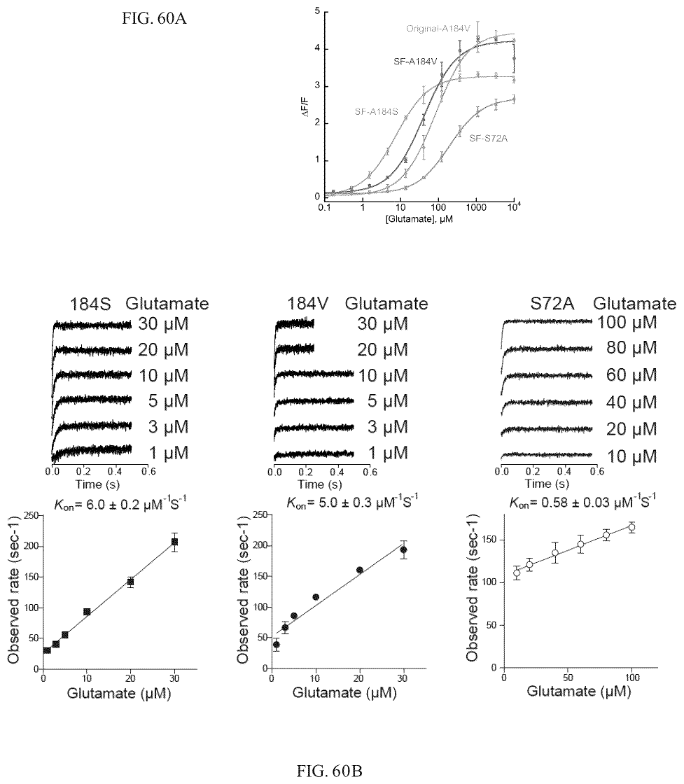

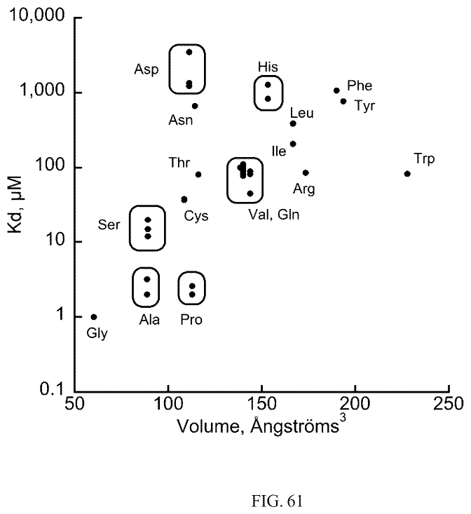

While the affinity of membrane-displayed iGluSnFR (4 μM) is adequate for some in vivo applications, tighter variants are needed for circumstances of limiting glutamate concentrations, such as at sparsely-firing synapses. Additionally, measuring glutamate release events with raster scanning microscopes requires variants with slower off-rates so that the decay time from glutamate binding is long enough to be sufficiently sampled at the operating frame rate for most experiments (typically <100 Hz). Replacement of eGFP with superfolder GFP increases the in vitro affinity of soluble SF-iGluSnFR two-fold compared to original iGluSnFR (40 μM vs. 80 FIG. 60a). To further modulate affinity, the conformational coupling between the open-closed equilibrium of bacterial periplasmic binding proteins (PBPs, e.g. the glutamate-binding protein in iGluSnFR) and their ligand-binding affinity (Marvin et al., 2001, Nat. Struct. Biol., 8:795-8) was exploited. Briefly, mutation of residues in the “hinge” of PBPs can allosterically alter affinity, without compromising the stereochemical integrity of the ligand-binding site. In a bacterial lysate assay, an A184X library of the iGluSnFR glutamate-binding domain (mutated to valine in the original iGluSnFR) was screened. Reversion to alanine or other small amino acids tightened affinity, while larger side chains weakened affinity (FIG. 61).

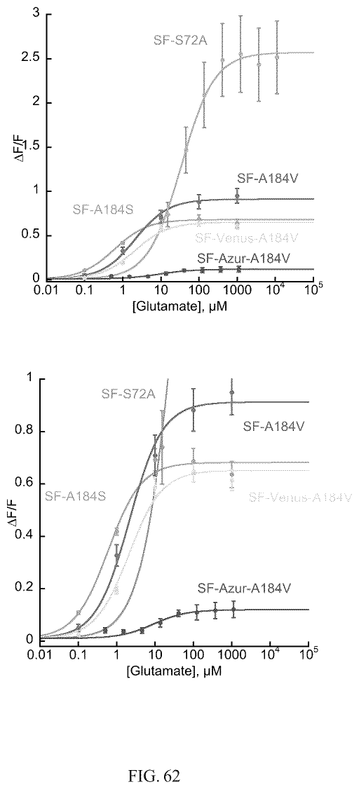

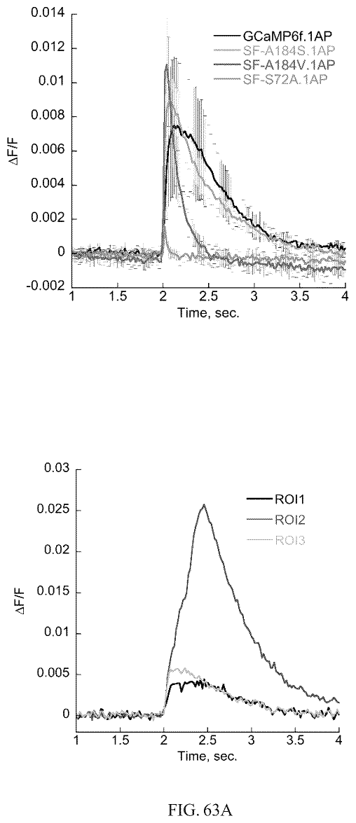

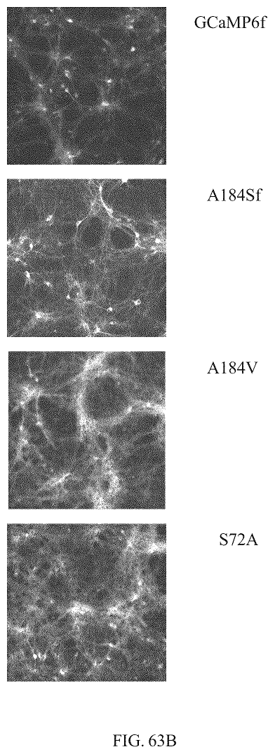

A184S was introduced into SF-iGluSnFR to generate a tighter variant. (Reversion A184A had a low ΔF/F.) Affinities of purified soluble protein were 7 μM and 40 μM for the A184S and A184V (unmutated from iGluSnFR) SF-iGluSnFR variants, respectively (FIG. 60a). The tighter affinity of the A184S variant arises from a slower off-rate (FIG. 60b). The affinity variants were re-cloned into an AAV vector containing an IgG secretion signal and a PDGFR transmembrane domain. Viral expression on cultured rat hippocampal neurons (AAV2/1.hSynapsin1.SF-iGluSnFR) yields glutamate affinities about an order of magnitude tighter than the soluble form (0.7 μM and 2 μM for A184S and A184V, respectively; FIG. 62). A similar increase in affinity upon membrane tethering was seen with the original sensor (Marvin et al., 2013, Nat. Methods, 10:162-70). Whole-field stimulation (50 Hz) of these cultures shows that their relative half-times of fluorescence decay parallel their in vitro kinetics, with all variants having faster decay than GCaMP6f (FIG. 63).

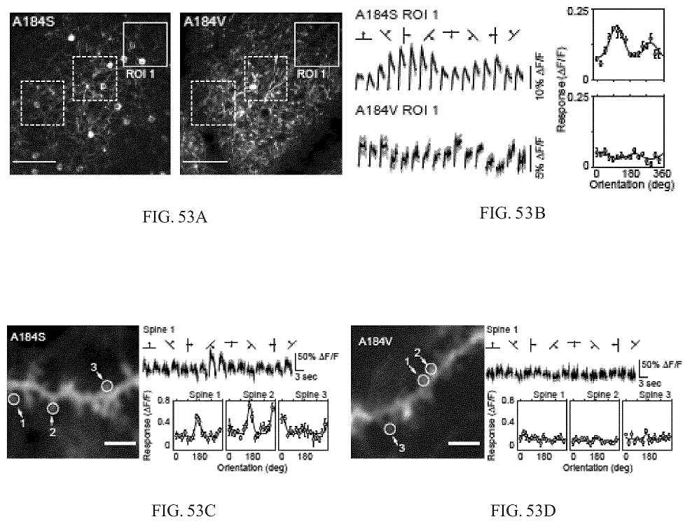

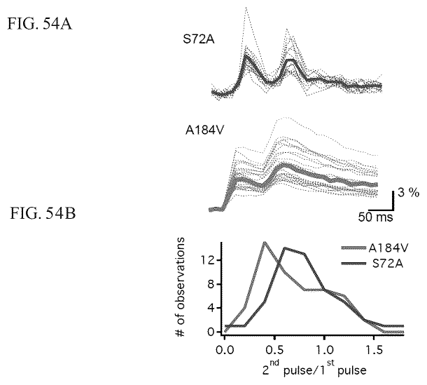

In vivo, the tighter/slower SF-iGluSnFR.A184S variant shows improved detection of stimulus-evoked glutamate release in the ferret visual cortex in response to presented drifting gratings (FIG. 53a,b). Peak amplitudes reached 30% ΔF/F for SF-iGluSnFR.A184S but only 5% ΔF/F for SF-iGluSnFR.A184V when imaged at 30 Hz. Greater ΔF/F of SF-iGluSnFR.A184S allows extraction of robust orientation tuning curves compared to SF-iGluSnFR.A184V. Enhanced sensitivity of the A184S variant also allowed orientation-selective responses to be resolved in individual dendritic spines (FIG. 53c,d). Synaptic glutamate release as measured with SF-iGluSnFR.A184S was not only strongly selective for visual stimuli, but response amplitudes across individual trials were consistently greater than the A184V variant when examining all stimulus-evoked responses (A184S median ΔF/F=16%, n=72 spines; A184V median ΔF/F=9%, n=22 spines; p=2e-115, Wilcoxon rank-sum test) or only preferred stimuli (A184S median ΔF/F=27%, n=72 spines; A184V median ΔF/F=14%, n=22 spines; p=9e-23, Wilcoxon rank-sum test) (FIG. 64).

While slow off-rate variants of SF-iGluSnFR are better for detecting individual synaptic events by temporal summation of fluorescence, faster off-rate variants are needed for temporal resolution spiking dynamics and at large synapses where glutamate clearance is limiting. A weaker variant of SF-iGluSnFR (S72A) was made by removing a hydrogen bond between the protein and glutamate. Soluble SF-iGluSnFR.S72A has 200 μM affinity for glutamate (FIG. 60a), arising from a combination of both slower on-rate and faster off-rate (FIG. 60b). In neuronal culture, S72A has an affinity of 35 μM, an order of magnitude weaker than its parent, A184V (FIG. 62).

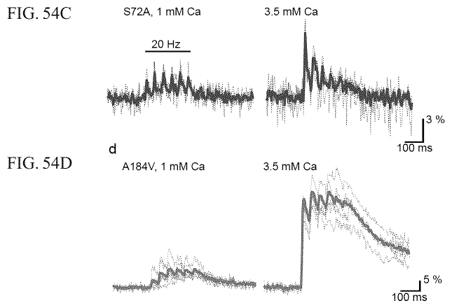

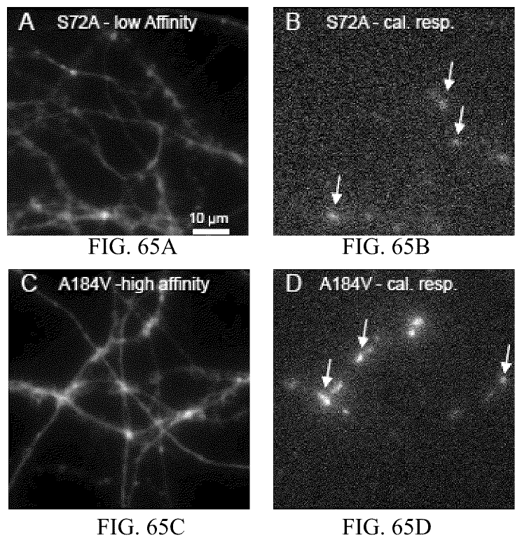

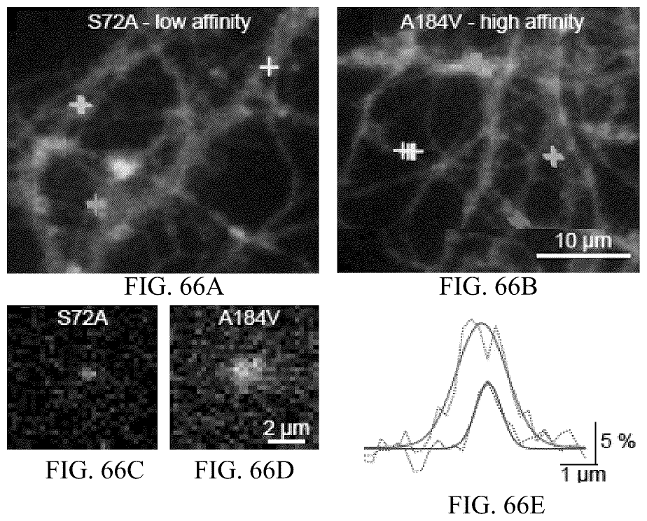

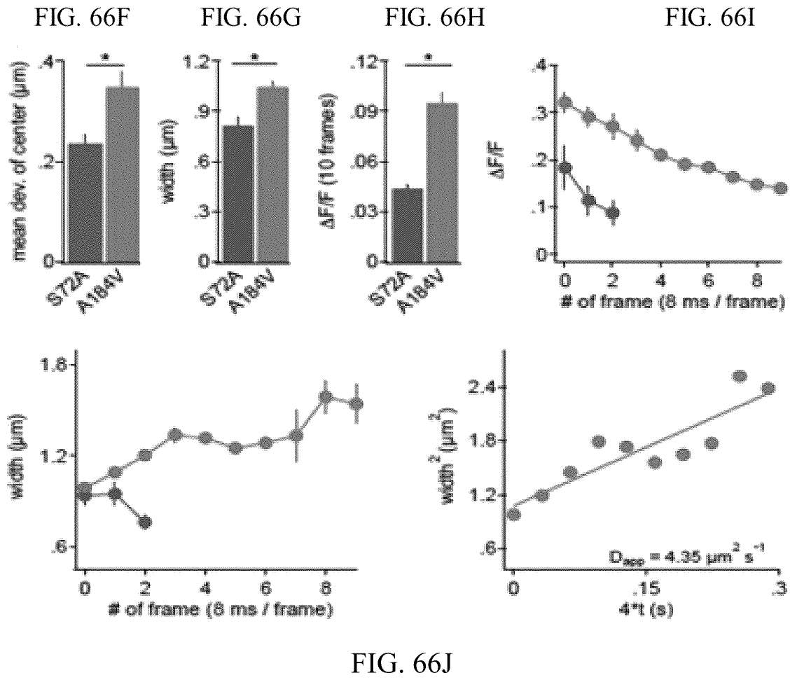

In rat neuronal culture, without buffer perfusion, fluorescence of the culture (not localized to specific structures) returns to baseline within 100 msec. of a single electrical stimulation for S72A, faster than A184V, A184S, or GCaMP6f (FIG. 63). In mouse neuronal culture (FIG. 65), the substantially faster off-rate of S72A provides enhanced temporal resolution of paired (20 Hz) electrical stimuli over the A184V variant (FIG. 54a,b), making it useful for assessing short-term synaptic plasticity. A train of 6 electrical pulses (20 Hz) in 1 mM extracellular Ca²⁺ can be resolved as equal, individual release events by observation with S72A, while A184V yields an integrated signal (FIG. 54c,d). In 3.5 mM extracellular Ca²⁺, vesicles are released with higher probability during the initial stimulation (Dodge et al., 1967, J. Physiol., 193:419-32). This can be observed by S72A, as reported by a reduction in fluorescence response as the train of field pulses progresses (FIG. 54c), while these differences are obscured by the slower decay of A184V (FIG. 54d). Thus, while S72A has a lower ΔF/F in response to the same amount of glutamate being released (due to weaker affinity), its faster kinetics provides enhanced temporal resolution of synaptic activity. Similarly, S72A provides enhanced spatial resolution of glutamate release over A184V (FIG. 66).

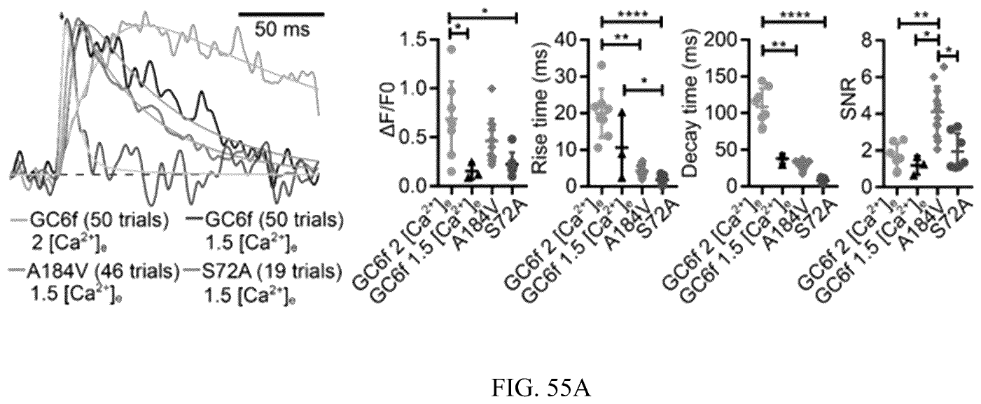

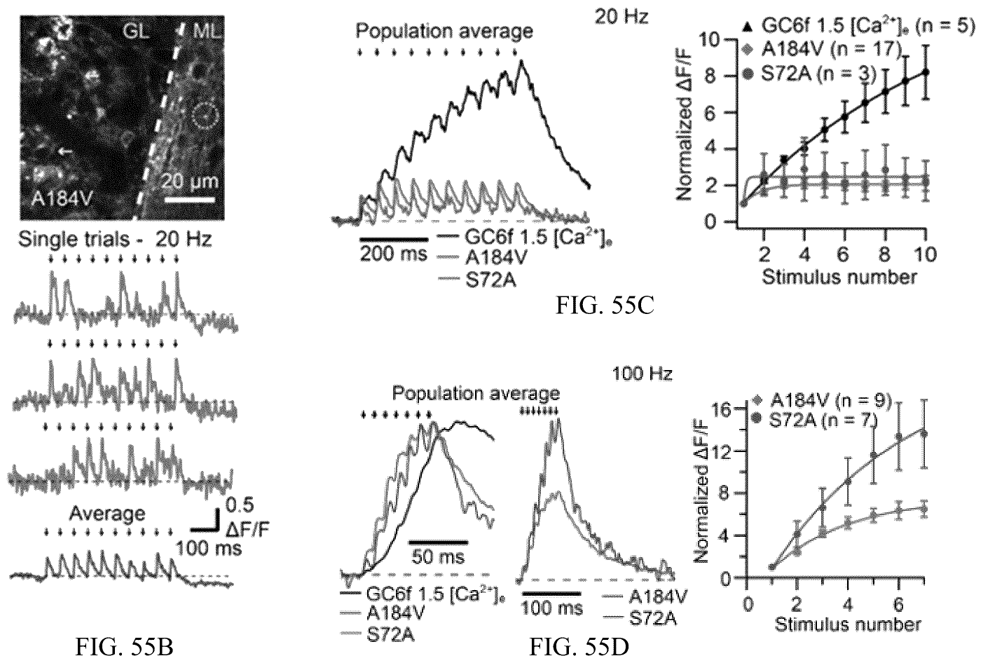

With fast rise and decay times, it was examined whether SF-iGluSnFR could be used as an alternative to GCaMP6f for monitoring neuronal activity in mouse cerebellar brain slice. Single cerebellar granule cell bouton responses to single action potentials (APs) could indeed be resolved using fast linescan detection (<1 ms per line; FIG. 55a), and were much faster than GCaMP6f rise and decay times at both 2 mM and 1.5 mM extracellular calcium. The S72A variant had by far the fastest response (S72 half decay 7.9±1.0 ms, A184V 28.1±1.6 ms, GCaMP6f 1.5 mM [Ca²⁺]ₑ 37.9±3.9 ms, GCaMP6f 1.5 mM [Ca²⁺]ₑ 108.6±8.8 ms). The signal-to-noise-ratios (SNRs) were best for A184V, but even S72A produced better SNRs than GCaMP6f under physiological extracellular calcium concentrations (1.5 mM). The superior SNR of A184V showed putative single vesicle release events in single trials (FIG. 55b). However, if many bouton responses are pooled and averaged for each trial, single spike detection at 20 Hz is feasible (see average trace, FIG. 55b). For 20 Hz stimuli, both the A184V and S72A variants produced little accumulation of bouton fluorescence after 10 stimuli as compared to GCaMP6f (FIG. 55c), similar to the dendritic responses in culture (FIG. 54). For 100 Hz train stimuli, discrete release events could be detected, in contrast to GCaMP6f (FIG. 55d). Note the poor temporal precision of the train response, in contrast to A184V and S72A. Thus both A184V and S72A enable a larger dynamic range of reported firing frequencies, with S72A providing the largest range due to its low affinity. Moreover, the fast kinetics of SF-iGluSnFR.A184V and SF-iGluSnFR.S72A could be used for a more reliable estimate of spike times (versus GCaMP6f), and are much better suited to high-frequency spike detection (>100 Hz) which is necessary for the high instantaneous firing rates of cerebellar granule cells (van Beugen et al., 2013, Frontiers in Neural Circuits, 7:95).

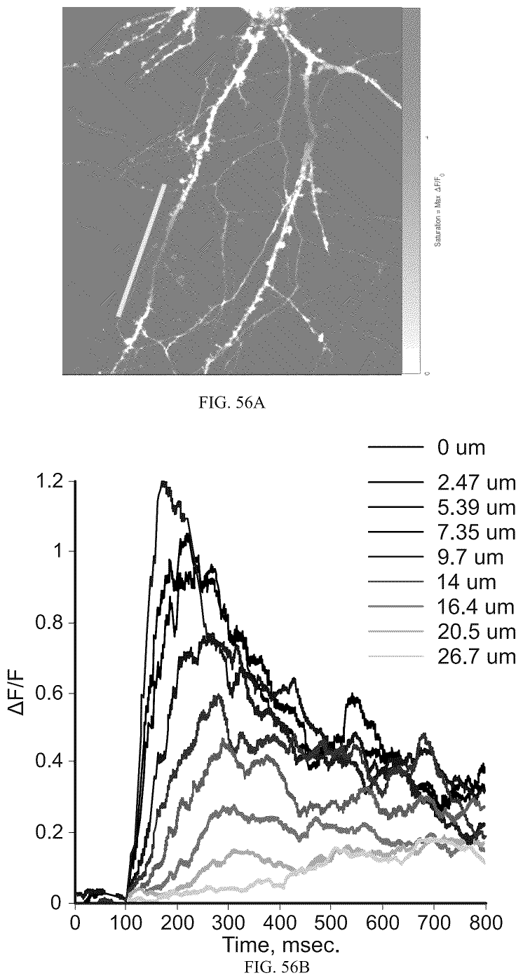

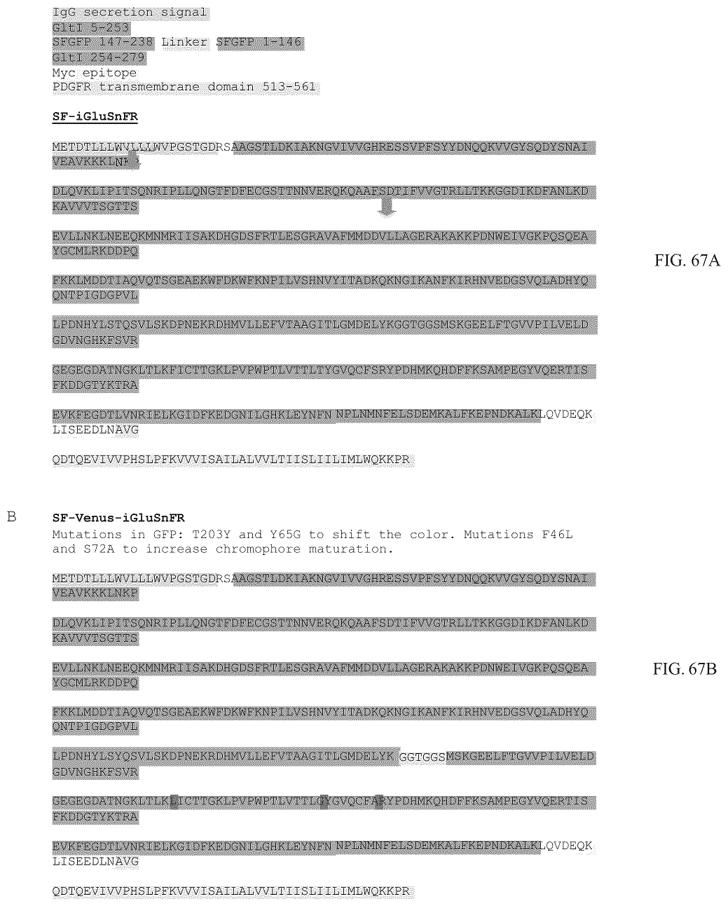

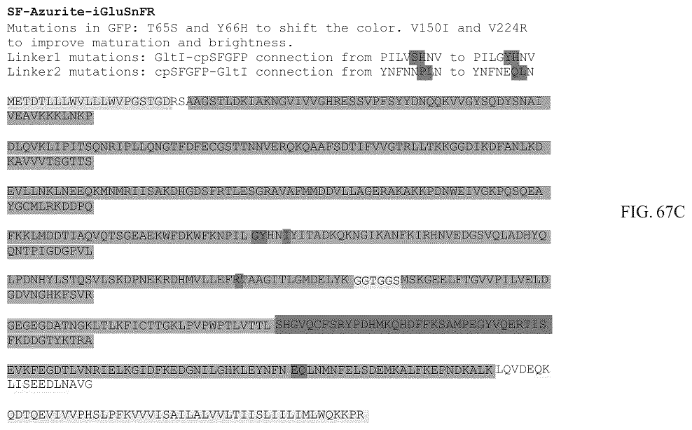

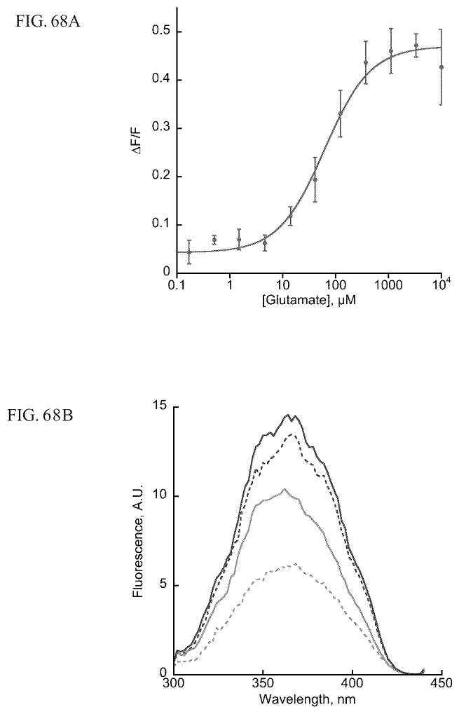

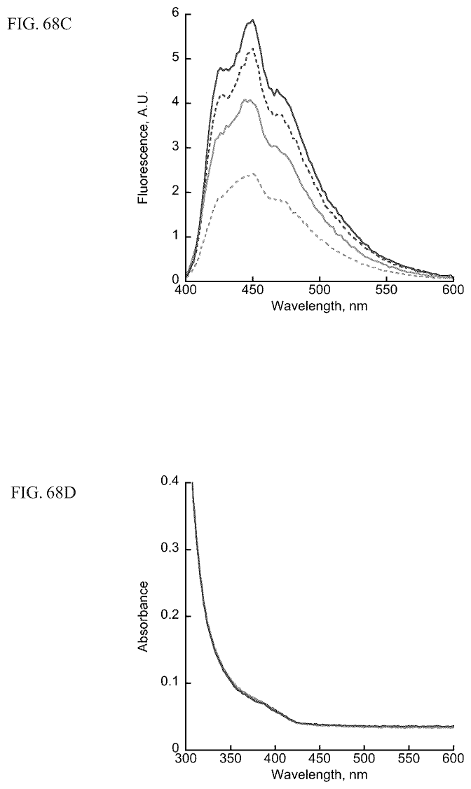

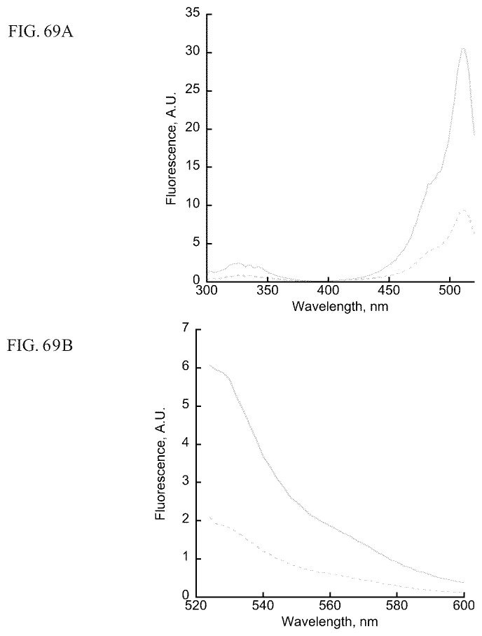

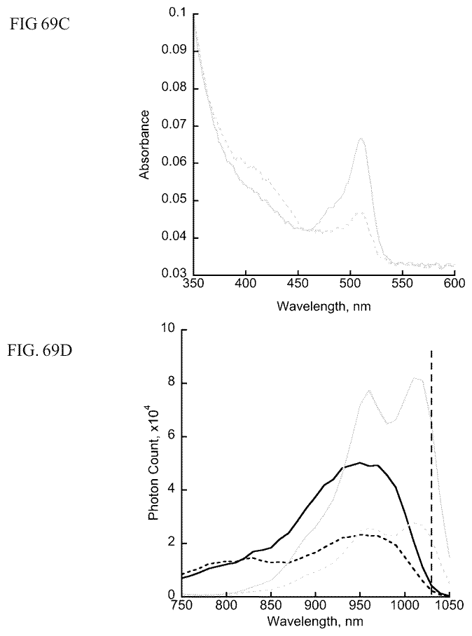

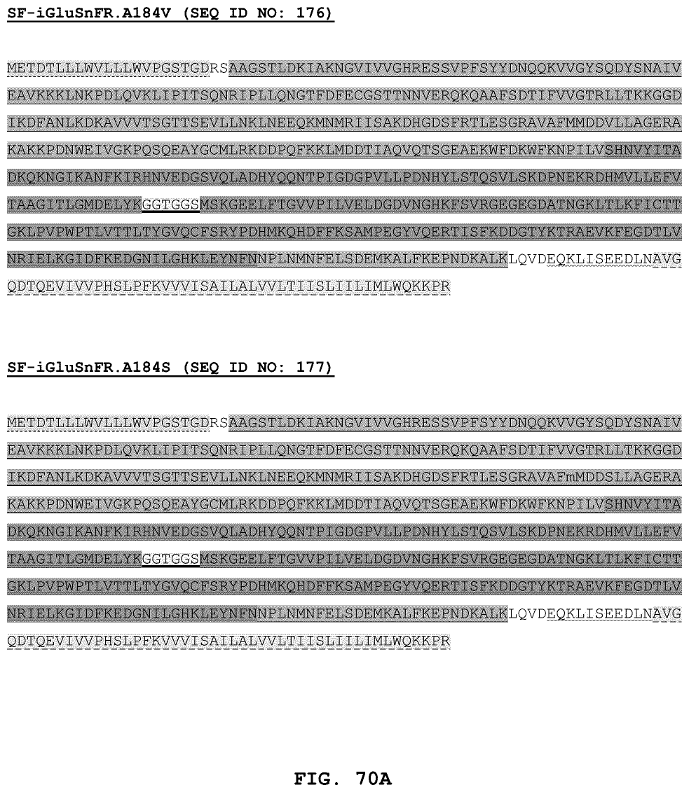

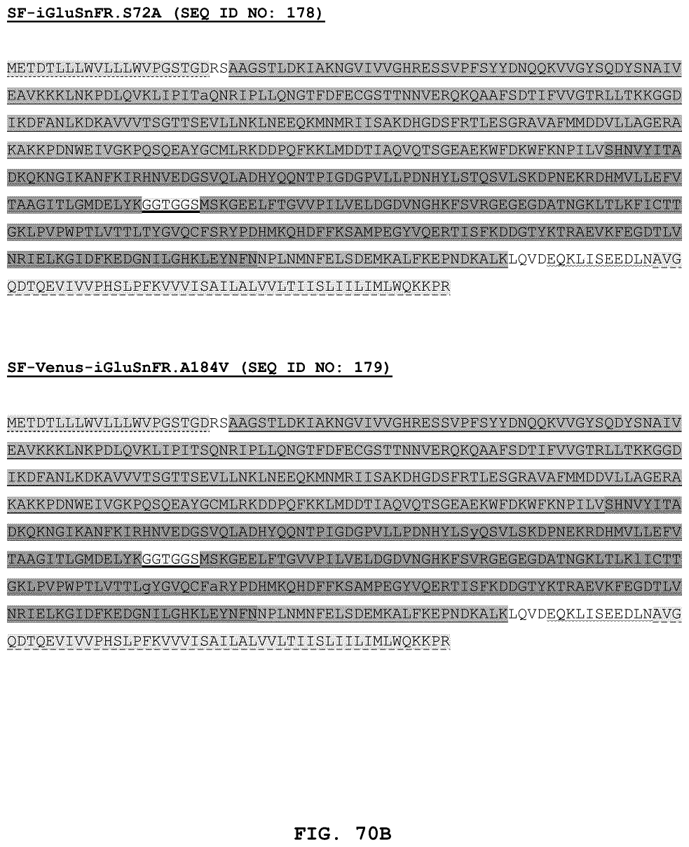

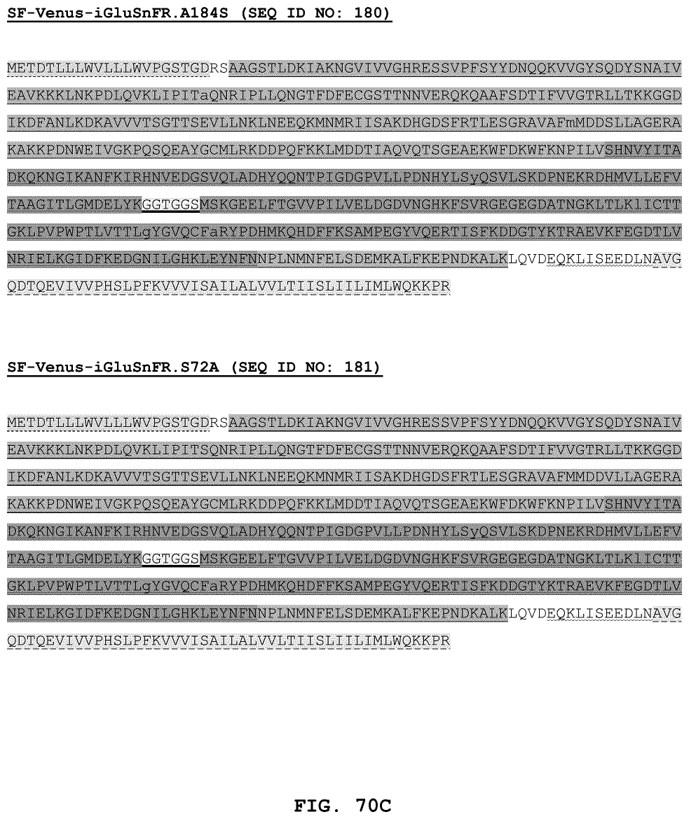

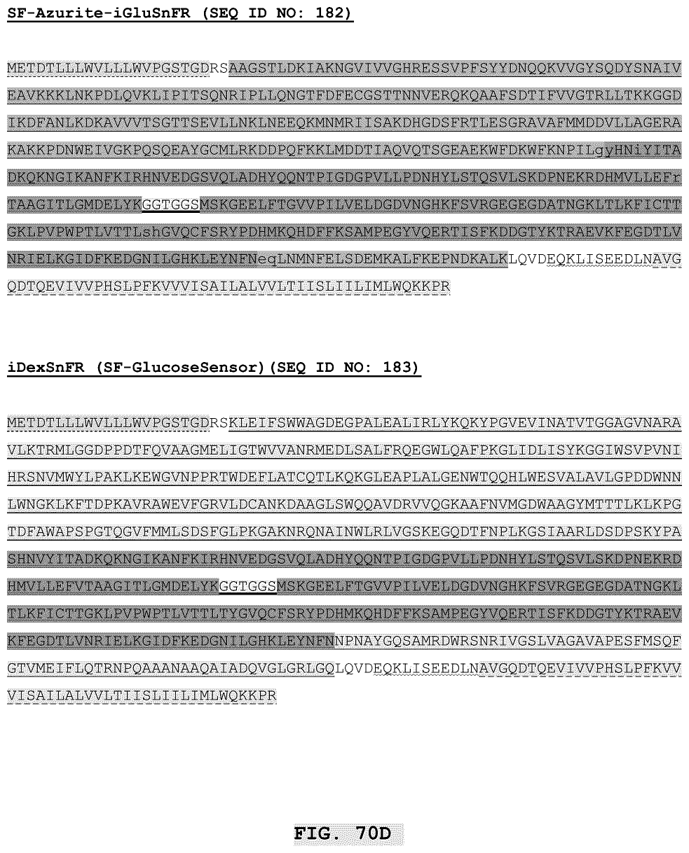

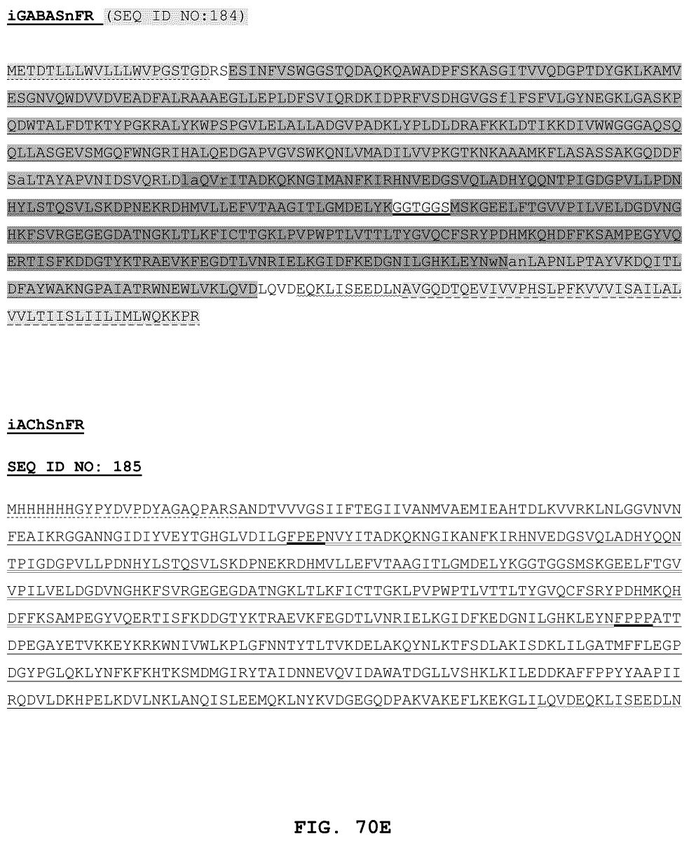

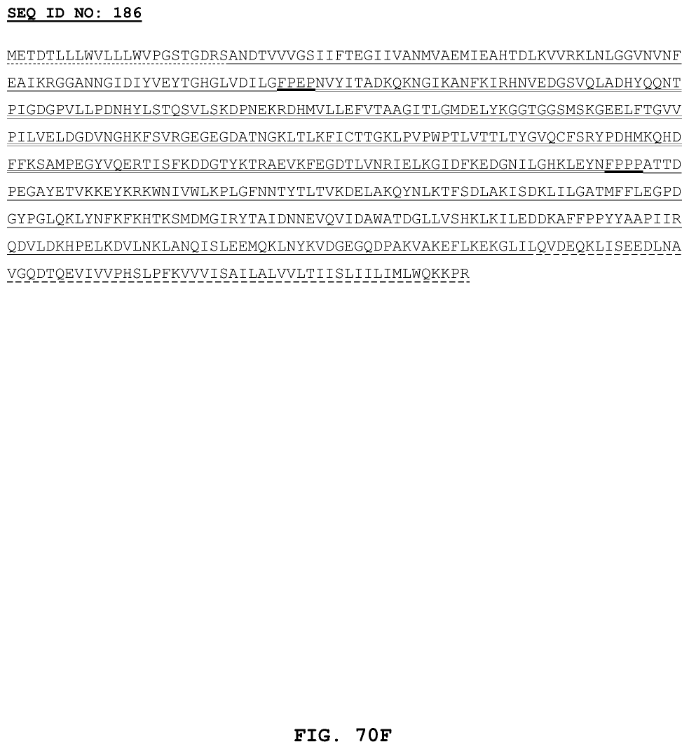

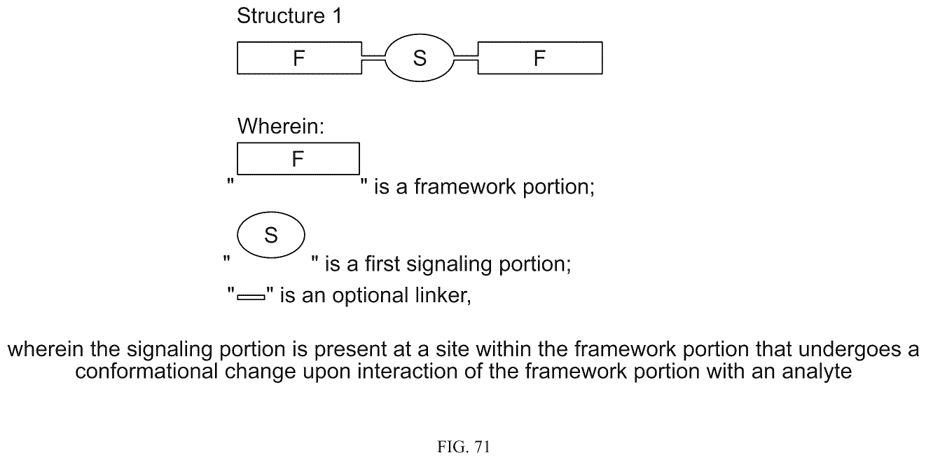

Introduction of chromophore mutations from GFP variants Azurite (Mena et al., 2006, Nat. Biotech., 24:1569-71) or Venus (Nagai et al., 2002, Nat. Biotech., 20:87-90) to SF-iGluSnFR led to functional blue and yellow versions, respectively. The former required re-optimization of the residues that link the FP with the glutamate-binding protein. The latter was a straightforward modular replacement. (Annotated amino acid sequences are given in FIG. 67). SF-Azurite-iGluSnFR has significantly lower ΔF/F (FIG. 68), perhaps a result of intrinsic differences in chromophore structure. SF-Venus-iGluSnFR has similar affinity and maximum fluorescence response to glutamate as SF-iGluSnFR, but with red-shifted excitation and emission spectra (FIG. 69). Importantly, its 2-photon excitation spectrum is sufficiently red-shifted to allow strong excitation at 1030 nm (FIG. 69), compatible with relatively inexpensive, powerful femtosecond fiber lasers (Tang et al., 2009, J. Biomed. Optics, 14:030508). These powerful lasers enable simultaneous excitation of many foci, enabling very fast (1016 Hz) large-area imaging by recording projections of the sample and computationally reconstructing images (Kazemipour, et al., 2018). In neuronal culture, two near-simultaneous pulses of glutamate uncaging can be resolved with both high spatial and temporal resolution by measuring fluorescence changes in a neuron expressing SF-Venus-iGluSnFR.A184V (FIG. 56).

The iGluSnFR variants described here increase the power of genetically encoded glutamate imaging. Affinity variants with altered kinetics broaden the range of observable glutamate release events. Chromatic mutants allow fast imaging with cheap lasers, and potential utility in multi-color imaging. Improved membrane targeting and photostability will be valuable in all applications.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A recombinant peptide biosensor comprising an analyte-binding framework portion and a signaling portion, wherein the signaling portion is present within the framework portion at a site or amino acid position that undergoes a conformational change upon interaction of the framework portion with a defined, specific, or selected analyte, wherein the recombinant peptide biosensor comprises a amino acid sequence having at least 85% sequence identity to SEQ ID NO: 179.

2. The recombinant peptide biosensor of claim 1, wherein the recombinant peptide biosensor comprises an amino acid sequence having at least 90% sequence identity to SEQ ID NO:179.

3. The recombinant peptide biosensor of claim 1, wherein the recombinant peptide biosensor comprises an amino acid sequence having at least 95% sequence identity to SEQ ID NO:179.

4. The recombinant peptide biosensor of claim 1, wherein the recombinant peptide biosensor comprises an amino acid sequence having at least 99% sequence identity to SEQ ID NO:179.

5. The recombinant peptide biosensor of claim 1, wherein the recombinant peptide biosensor comprises an amino acid sequence of SEQ ID NO:179.

6. The recombinant peptide biosensor of claim 1, wherein signaling by the signaling portion detectably increases upon interaction of the framework portion with the analyte.

7. The recombinant peptide biosensor of claim 1, wherein signaling by the signaling portion detectably decreases upon interaction of the framework portion with the analyte.

8. The recombinant peptide biosensor of claim 1, wherein signaling by the signaling portion is proportional to the level of interaction between the framework portion and the analyte.

9. The recombinant peptide biosensor of claim 1, wherein the framework portion is a periplasmic binding protein (PBP) or a variant of a PBP.

10. The recombinant peptide biosensor of claim 1, wherein the signaling portion is a circularly permuted super fluorescent (SF) protein.

11. The recombinant peptide biosensor of claim 9, wherein the SF protein is selected from the group consisting of a green fluorescent protein, a yellow fluorescent protein, a red fluorescent protein, and a blue fluorescent protein.

12. The recombinant peptide biosensor of claim 1, wherein the analyte-binding framework portion binds specifically to glutamate.

13. The recombinant peptide biosensor of claim 1, comprising 10 or fewer conservative amino acid substitutions.

14. A nucleic acid encoding the recombinant peptide biosensor of claim 1.

15. A vector comprising the nucleic acid of claim 14.

16. A cell comprising the nucleic acid of claim 14.

17. A cell comprising the vector of claim 15.

18. A kit comprising the recombinant peptide biosensor of claim 1, the nucleic acid of claim 14, the vector of claim 15, or the cell of claim 16 or claim 17.

19. A recombinant peptide biosensor comprising an analyte-binding framework portion and a signaling portion, wherein the signaling portion is present within the framework portion at a site or amino acid position that undergoes a conformational change upon interaction of the framework portion with a defined, specific, or selected analyte, wherein the recombinant peptide biosensor comprises an amino acid sequence having at least 85% sequence identity to SEQ ID NO: 180.

20. The recombinant peptide biosensor of claim 19, wherein the recombinant peptide biosensor comprises an amino acid sequence having at least 90% sequence identity to SEQ ID NO:180.

21. The recombinant peptide biosensor of claim 19, wherein the recombinant peptide biosensor comprises an amino acid sequence having at least 95% sequence identity to SEQ ID NO:180.

22. The recombinant peptide biosensor of claim 19, wherein the recombinant peptide biosensor comprises an amino acid sequence having at least 99% sequence identity to SEQ ID NO:180.

23. The recombinant peptide biosensor of claim 19, wherein the recombinant peptide biosensor comprises an amino acid sequence of SEQ ID NO:180.

24. A recombinant peptide biosensor comprising an analyte-binding framework portion and a signaling portion, wherein the signaling portion is present within the framework portion at a site or amino acid position that undergoes a conformational change upon interaction of the framework portion with a defined, specific, or selected analyte, wherein the recombinant peptide biosensor comprises an amino acid sequence having at least 85% sequence identity to SEQ ID NO: 181.

25. The recombinant peptide biosensor of claim 24, wherein the recombinant peptide biosensor comprises an amino acid sequence having at least 90% sequence identity to SEQ ID NO:181.

26. The recombinant peptide biosensor of claim 24, wherein the recombinant peptide biosensor comprises an amino acid sequence having at least 95% sequence identity to SEQ ID NO:181.

27. The recombinant peptide biosensor of claim 24, wherein the recombinant peptide biosensor comprises an amino acid sequence having at least 99% sequence identity to SEQ ID NO:181.

28. The recombinant peptide biosensor of claim 24, wherein the recombinant peptide biosensor comprises an amino acid sequence of SEQ ID NO:181.
